SEQUENCE LISTING (29 sequences)

SEQ ID NO: 1 - protein, 5 aa, Artificial Sequence
  Linker
  REPEAT (1)..(5): repeat n times, where n represents an integer of at least 1
GSGGS

SEQ ID NO: 2 - protein, 4 aa, Artificial Sequence
  Linker
  REPEAT (1)..(4): repeat n times, where n represents an integer of at least 1
GGGS

SEQ ID NO: 3 - protein, 5 aa, Artificial Sequence
  Linker
  REPEAT (1)..(5): repeat n times, where n represents an integer of at least 1
GGGGS

SEQ ID NO: 4 - protein, 4 aa, Artificial Sequence
  Linker
GGSG

SEQ ID NO: 5 - protein, 5 aa, Artificial Sequence
  Linker
GGSGG

SEQ ID NO: 6 - protein, 5 aa, Artificial Sequence
  Linker
GSGSG

SEQ ID NO: 7 - protein, 5 aa, Artificial Sequence
  Linker
GSGGG

SEQ ID NO: 8 - protein, 5 aa, Artificial Sequence
  Linker
GGGSG

SEQ ID NO: 9 - protein, 5 aa, Artificial Sequence
  Linker
GSSSG

SEQ ID NO: 10 - protein, 5 aa, Artificial Sequence
  Linker
GGGGS

SEQ ID NO: 11 - protein, 15 aa, Artificial Sequence
  Linker
GGGGSGGGGSGGGGS

SEQ ID NO: 12 - DNA, 45 bp, Artificial Sequence
  Linker
ggtggcggtggctcgggcggtggtgggtcgggtggcggcggatct

SEQ ID NO: 13 - protein, 5 aa, Artificial Sequence
  Hinge
DKTHT

SEQ ID NO: 14 - protein, 4 aa, Artificial Sequence
  Hinge
CPPC

SEQ ID NO: 15 - protein, 15 aa, Artificial Sequence
  Hinge
CPEPKSCDTPPPCPR

SEQ ID NO: 16 - protein, 12 aa, Artificial Sequence
  Hinge
ELKTPLGDTTHT

SEQ ID NO: 17 - protein, 10 aa, Artificial Sequence
  Hinge
KSCDKTHTCP

SEQ ID NO: 18 - protein, 7 aa, Artificial Sequence
  Hinge
KCCVDCP

SEQ ID NO: 19 - protein, 7 aa, Artificial Sequence
  Hinge
KYGPPCP

SEQ ID NO: 20 - protein, 15 aa, Artificial Sequence
  Hinge
EPKSCDKTHTCPPCP

SEQ ID NO: 21 - protein, 12 aa, Artificial Sequence
  Hinge
ERKCCVECPPCP

SEQ ID NO: 22 - protein, 17 aa, Artificial Sequence
  Hinge
ELKTPLGDTTHTCPRCP

SEQ ID NO: 23 - protein, 12 aa, Artificial Sequence
  Hinge
SPNMVPHAHHAQ

SEQ ID NO: 24 - protein, 15 aa, Artificial Sequence
  Hinge
EPKSCDKTYTCPPCP

SEQ ID NO: 25 - protein, 4 aa, Artificial Sequence
  Furin cleavage site
  misc_feature (2)..(2): Xaa can be any naturally occurring amino acid
RXKR

SEQ ID NO: 26 - protein, 4 aa, Artificial Sequence
  Furin cleavage site
  misc_feature (2)..(2): Xaa can be any naturally occurring amino acid
RXRR

SEQ ID NO: 27 - protein, 5 aa, Artificial Sequence
  Furin cleavage site
  misc_feature (1)..(1): Xaa is Lys or Arg
  misc_feature (3)..(3): Xaa can be any naturally occurring amino acid
  misc_feature (4)..(4): Xaa is Lys or Arg
XRXXR

SEQ ID NO: 28 - protein, 4 aa, Artificial Sequence
  Furin cleavage site
  misc_feature (2)..(3): Xaa can be any naturally occurring amino acid
RXXR

SEQ ID NO: 29 - protein, 4 aa, Artificial Sequence
  Furin cleavage site
RQKR

(19) United States
(12) Patent Application Publication
(10) Pub. No.: US 2021/0230246 A1
(43) Pub. Date: Jul. 29, 2021

(54) USE OF INHIBITORY CHIMERIC RECEPTORS TO PREVENT T CELL-INDUCED BLOOD BRAIN BARRIER DAMAGE

(71) Applicants: THE TRUSTEES OF THE UNIVERSITY OF PENNSYLVANIA, Philadelphia, PA (US); THE BOARD OF TRUSTEES OF THE LELAND STANFORD JUNIOR UNIVERSITY, Stanford, CA (US)

(72) Inventors: Avery Posey, Philadelphia, PA (US); Carl H. June, Merion Station, PA (US); Denis Migliorini, Geneva (CH); Howard Chang, Stanford, CA (US); Ansuman Satpathy, Stanford, CA (US); Kevin Parker, Stanford, CA (US)

(21) Appl. No.: 17/149,541

(22) Filed: Jan. 14, 2021

Related U.S. Application Data
(60) Provisional application No. 62/961,408, filed on Jan. 15, 2020.
(60) Provisional application No. 63/080,531, filed on Sep. 18, 2020.

(51) Int. Cl. C07K 14/725; C07K 16/28; A61K 35/17

(52) U.S. Cl. CPC C07K 14/7051; C07K 16/2803; C07K 2317/53; C07K 2317/55; C07K 2317/622; A61K 35/17

(57) ABSTRACT

Neurotoxicity associated with CD19-targeted CAR-T therapy is reduced by including an inhibitory CAR (iCAR) in the CAR-T cell, wherein the iCAR specifically recognizes an antigen specific for or associated with neurovascular mural cells (e.g. pericytes or vSMC).

### CROSS-REFERENCE TO RELATED APPLICATIONS

The present application is entitled to priority under 35 U.S.C. § 119(e) to U.S. Provisional Patent Application No. 62/961,408 filed Jan. 15, 2020, and U.S. Provisional Patent Application No. 63/080,531 filed Sep. 18, 2020, each of which is hereby incorporated by reference in its entirety herein.

### SEQUENCE LISTING

The instant application contains a Sequence Listing which has been submitted in ASCII format via EFS-Web and is hereby incorporated by reference in its entirety. Said ASCII copy, created on Jan. 14, 2021, is named 046483-7266US1_SequenceListing_ST25.txt and is 6.30 kilobytes in size.

### BACKGROUND OF THE INVENTION

For patients with B-cell lymphoma, including those who have relapsed after treatment with current standard-of-care chemotherapy, new immunotherapies have shown tremendous clinical efficacy. In a recent phase II study of 111 patients with refractory B-cell lymphoma, of whom 101 were administered CD19 CAR-T cell therapy, 40% of patients showed a complete remission of disease 15 months after treatment. Similar results were observed in a separate study, with complete remission observed in 43% and 71% of patients with diffuse large B-cell and follicular lymphoma, respectively. However, in addition to adverse effects related to cytokine release syndrome (CRS), both studies reported a high incidence of neurotoxicity (64% and 39%, respectively). These findings are in agreement with previously reported rates of neurotoxicity in patients receiving CD19 CAR-T cell therapy and CD19/CD3 BiTE® therapy. This neurotoxicity is often severe, including cases of fatal cerebral edema associated with T cell infiltration into the brain. The pathophysiology of this neurotoxicity remains unclear, yet an understanding of its etiology and progression is critical for the development of safer CAR and BiTE® therapies.

Thus, there is an urgent need in the art for compositions and methods which reduce the neurotoxicity of CAR-T cell and BiTE® therapies, especially those targeting CD19+ hematologic malignancies. The present invention addresses that need.

### SUMMARY OF THE INVENTION

As described herein, the present invention relates to compositions and methods comprising an inhibitory CAR (iCAR) capable of binding a mural cell (e.g. a pericyte or vSMC).

One aspect of the invention includes a modified immune cell comprising a chimeric antigen receptor (CAR) and an inhibitory CAR (iCAR). The CAR comprises an antigen-binding domain capable of binding CD19, a transmembrane domain, and an intracellular domain. The iCAR comprises an antigen-binding domain capable of binding a pericyte-associated or pericyte-specific antigen, and an inhibitory signaling domain.

Another aspect of the invention includes a nucleic acid encoding an inhibitory chimeric antigen receptor (iCAR), wherein the iCAR comprises an antigen-binding domain capable of binding a pericyte-associated or pericyte-specific antigen, and an inhibitory signaling domain.

Yet another aspect of the invention includes a inhibitory chimeric antigen receptor (iCAR) comprising an antigen-binding domain capable of binding a pericyte-associated or a pericyte-specific antigen, and an inhibitory signaling domain.

Still another aspect of the invention includes a method of treating cancer in a subject in need thereof. The method comprises administering to the subject a modified T cell comprising a CAR and an iCAR. The CAR comprises an antigen-binding domain capable of binding CD19, a transmembrane domain, and an intracellular domain. The iCAR comprises an antigen-binding domain capable of binding a pericyte-associated or pericyte-specific antigen, and an inhibitory signaling domain.

Another aspect of the invention includes a method of inhibiting CAR T cell-induced neurotoxicity. The method comprises administering to the subject an effective amount of a modified T cell comprising a CAR and an iCAR. The CAR comprises an antigen-binding domain capable of binding CD19, a transmembrane domain, and an intracellular domain. The iCAR comprises an antigen-binding domain capable of binding a pericyte-associated or pericyte-specific antigen, and an inhibitory signaling domain.

Another aspect of the invention includes a modified immune cell comprising a chimeric antigen receptor (CAR) and an inhibitory CAR (iCAR). The CAR comprises an antigen-binding domain capable of binding CD19, a transmembrane domain, and an intracellular domain. The iCAR comprises an antigen-binding domain capable of binding a mural cell-associated or mural cell-specific antigen, and an inhibitory signaling domain.

Another aspect of the invention includes a nucleic acid encoding an inhibitory chimeric antigen receptor (iCAR), wherein the iCAR comprises an antigen-binding domain capable of binding a mural cell-associated or mural cell-specific antigen, and an inhibitory signaling domain.

Yet another aspect of the invention includes a inhibitory chimeric antigen receptor (iCAR) comprising an antigen-binding domain capable of binding a mural cell-associated or a mural cell-specific antigen, and an inhibitory signaling domain.

Still another aspect of the invention includes a method of treating cancer in a subject in need thereof. The method comprises administering to the subject a modified T cell comprising a CAR and an iCAR. The CAR comprises an antigen-binding domain capable of binding CD19, a transmembrane domain, and an intracellular domain. The iCAR comprises an antigen-binding domain capable of binding a mural cell-associated or mural cell-specific antigen, and an inhibitory signaling domain.

Another aspect of the invention includes a method of inhibiting CAR T cell-induced neurotoxicity. The method comprises administering to the subject an effective amount of a modified T cell comprising a CAR and an iCAR. The CAR comprises an antigen-binding domain capable of binding CD19, a transmembrane domain, and an intracellular domain. The iCAR comprises an antigen-binding domain capable of binding a mural cell-associated or mural cell-specific antigen, and an inhibitory signaling domain.

In various embodiments of the above aspects or any other aspect of the invention delineated herein, the antigen-binding domain of the CAR and/or iCAR is an antibody or an antigen-binding fragment thereof. In certain embodiments, the antigen-binding fragment is a Fab or a scFv. In certain embodiments, the antigen-binding domain of the iCAR is an extracellular domain of a pericyte-associated or pericyte-specific ligand. In certain embodiments, the antigen-binding domain of the iCAR is an extracellular domain of a mural cell-associated or mural cell-specific ligand. In certain embodiments, the antigen-binding domain of the iCAR binds to an antigen expressed on the surface of a pericyte, wherein the antigen is not CD19. In certain embodiments, the antigen-binding domain of the iCAR binds to an antigen expressed on the surface of a mural cell, wherein the antigen is not CD19. In certain embodiments, the antigen-binding domain of the iCAR binds to an antigen selected from the group consisting of CD146, PDGFRB, RGS5, CSPG4, CD248, BGN, FN1, SEMA5A, PLXDC1, THY1, CDH6, TFPI, COL1A2, ITGA1, EDNRA, PCDH18, CDH11, AXL, NTM, TNFRSF1A, S1PR3, and F3.

In certain embodiments, the inhibitory signaling domain of the iCAR comprises an inhibitory signaling domain of an inhibitory protein or a portion thereof. In certain embodiments, the inhibitory protein is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

In certain embodiments, the iCAR further comprises a transmembrane domain and/or a hinge domain. In certain embodiments, the transmembrane domain is selected from the group consisting of PD-1, CTLA-4, ICOS, LAG-3, TIM3, 2B4, and BTLA.

In certain embodiments, the immune cell is a T cell. In certain embodiments, the T cell is autologous. In certain embodiments, the T cell is allogeneic.

In certain embodiments, administering the modified T cell comprising a CAR and an iCAR further prevents neurotoxicity in the subject. In certain embodiments, the subject is human.

Another aspect of the invention includes a bi-specific iCAR comprising a) a CAR comprising an antigen-binding domain capable of binding CD19, a transmembrane domain, an intracellular domain, and a CD3zeta domain, and b) an iCAR comprising an antigen-binding domain capable of binding a mural cell, a transmembrane domain, and an inhibitory signaling domain.

In certain embodiments, the inhibitory signaling domain of the iCAR is selected from the group consisting of PTPN6, LAIR1, PD-1, and/KIR2DL4.

### BRIEF DESCRIPTION OF THE DRAWINGS

The following detailed description of embodiments of the invention will be better understood when read in conjunction with the appended drawings. It should be understood, however, that the invention is not limited to the precise arguments and instrumentalities of the embodiments shown in the drawings.

FIGS. 1A-1H are a series of graphs illustrating identification of CD19 expressing pericytes in single-cell RNA-sequencing data. FIG. 1A depicts Uniform Manifold Approximation and Projection for Dimension Reduction (UMAR) of single-cell prefrontal cortex RNA-seq data. FIG. 1B depicts the neuronal, neural progenitor cell, and erythroid clusters were identified as shown and subsequently excluded from further analysis. Unit, counts per million (CRM). FIG. 1C depicts non-neuronal cells re-clustered and embedded using UMAP to more clearly distinguish populations. FIG. 1D illustrates cell populations identified by shown marker genes. Note that pericytes and endothelial cells are called as a single cluster but identifiable as separate populations by marker gene expression. Scale bar indicates CRM. FIG. 1E illustrates a population of CD248-positive pericytes that are also positive for the CAR-T target CD19 but negative for the B cell marker CD79A. FIG. 1F is a heatmap showing relative gene abundances for specific marker genes across identified cell clusters. Pericytes are identified by PDGFRB, CD248, RGS5, and FOXF2 expression, and specifically express CD19. FIG. 1G is a histogram depicting mean abundance of all genes in the human prefrontal cortex. Percentiles indicate the expression rank of genes relative to all detected genes. FIG. 1H depicts a recapitulation of CD19 expression in pericytes in a human ventral midbrain scRNA-seq dataset.

FIGS. 2A-2C are a series of images depicting immunohistochemistry for CD19 in human brain tissue. Representative immunohistochemistry staining for CD19 in human brain tissue is shown. FFPE samples were stained for CD19 with a clinical protocol. Representative staining is shown for the hippocampus, insula, temporal lobe, frontal lobe, parietal lobe, lower medulla, pons, and occipital lobe. Bar scale: 200 μm.

FIGS. 3A-3D are a series of plots illustrating the presence of CD19⁺ non-B cells in the brains of healthy C57BL/6J mice. CNS stromal fractions were analyzed by flow cytometry. FIG. 3A shows the live cell fraction with gating for the CD45⁺ (B cells) and CD45⁻ (pericytes) populations. CD19⁺ cells are present in the CD45⁻ population as shown, along with CD31⁺ endothelial cells (middle). CD19⁺ B220⁺ B-cells are found in the CD45⁺ fraction (right). FIG. 3B shows fluorescence minus one (FMO) control staining for CD19 in CD45⁻ cells (middle) and in CD45⁺ cells (right). FIGS. 3C and 3D are histograms showing CD19 (FIG. 3C) and B220 (FIG. 3D) staining for the indicated populations.

FIGS. 4A-4H are a series of graphs and images illustrating the finding that CD19-directed CAR-T cells cause blood brain barrier disruption in immunodeficient mice. FIG. 4A shows a sequence alignment of the targeted human CD19 epitope with the corresponding murine CD19 region displaying low sequence homology. FIG. 4B depicts representation of CAR constructs and an animal study design using NSG mice. FIG. 4C depicts representative immunofluorescence imaging showing nuclear staining (DAPI) and Evans Blue dye (EBD) extravasation for BBB disruption in NSG mice following murine-targeting CD19 CAR-T treatment (n=5 mice per treatment). Scale bar, 75 m. FIG. 4D shows quantification of EBD staining in FIG. 4C in each group of mice as determined by the product of the mean fluorescence intensity (using Image J version 1.46: Image/Adjust/Color Threshold followed by Analyze/Measure) per surface area (μm). Representative sections (n=3 per mouse) were analyzed and graphed. Note the increased blood brain barrier leakiness in the CD28-based CAR-T cells versus the 4-1BB-based CAR-T cells. *p<0.05, **p<0.005, 2 tailed unpaired t-test.

FIG. 4E illustrates the finding that the syngeneic C57BL/6J mouse model recapitulates the murine CD19-specific neurotoxicity pattern observed in the immunodeficient model. Mice were treated with cyclophosphamide as a lymphodepleting regimen and injected with indicated CAR-T cells. EBD extravasation and nuclear staining (DAPI) are shown (n=5 mice per group) as in FIG. 4D. FIG. 4F shows quantification of EBD staining for each group shown in FIG. 4E. *p<0.05, **p<0.005, 2 tailed unpaired t-test. FIG. 4G depicts in vivo imaging using high-resolution MRI with gadolinium contrast showing blood brain barrier leakage after murine-targeting CD19 CAR-T cell therapy. All experiments were performed 4 days after CAR-T cell infusion. FIG. 4H depicts quantification of the mean signal intensity shown in FIG. 4G. Error bars indicate standard deviation.

FIGS. 5A-5C are a series of graphs demonstrating further analysis of single-cell RNA-sequencing data from human brain tissue. FIG. 5A is a heatmap showing gene-normalized abundances for specific marker genes across identified cell clusters. Pericytes are identified by PDGFRB, CD248, RGS5, and FOXF2 expression, and specifically express CD19. FIG. 5B depicts analysis of single cell RNA-seq data from human forebrain (La Manno et al., 2018, Nature 560, 494-498) showing CD19 expression in pericytes. FIG. 5C is a pair of histograms of mean gene expression values (log 10 counts per million) in identified pericyte cells in La Manno et al., 2016, Cell 167, 566-580.e19 and La Manno et al., 2018, Nature 560, 494-498. Relative gene expression percentiles are shown for indicated genes.

FIGS. 6A-6C are graphs depicting that anti-human and anti-mouse CD19-specific CARs show reactivity against human and mouse B-ALL cell lines respectively. FIG. 6A shows flow cytometry analysis of CAR expression 5 days after transduction of the hCD19BBz, mCD19BBz and mCD1928z CARs. FIG. 6B depicts results from flow cytometry based killing assays in human B-ALL cell line Nalm6 and murine CD19⁺ A20 B-ALL cell line in the presence of human 4-1BB or CD28 based anti-human or murine CD19 CAR-T cells. FIG. 6C shows IFN-7 expression measured by ELISA under the same conditions as in FIG. 6B, showing the species-specificity of the CAR constructs used.

FIGS. 7A-7C illustrate confirmation of mural cell CD19 expression in two independent datasets. FIGS. 7A-7B are UMAP plots showing single cell RNA-seq data from (FIG. 7A) human forebrain (La Manno et al., 2018) and (FIG. 7B) human ventral midbrain (La Manno et al., 2016), showing CD19 expression in pericytes. FIG. 7C is set of a histograms of mean gene expression values (log 10 counts per million) in identified pericyte cells in La Manno et al. 2018 (top) and La Manno et al. 2016 (bottom). Relative gene expression percentiles are shown for indicated genes.

FIGS. 8A-8I illustrate meta-cell clustering identifies CD19 expression in human neurovascular meta-cells. FIG. 8A is a schematic showing how samples were processed (see FIG. 13A for an example of a single dataset). FIG. 8B shows UMAP projection of meta-cells. FIGS. 8C-8D show expression of (FIG. 8C) CD19, (FIG. 8D) PAX3, marking undifferentiated progenitors, and mean expression of indicated pericyte marker genes. FIG. 8E is a histogram of mean mural cell marker expression across all samples, showing separation of the identified neurovascular cell cluster. FIG. 8F is a histogram as in (FIG. 8E), but separated by sample age, showing (left) pericyte marker gene expression, (middle) CD19 expression, (right) B cell marker gene expression. Note that CD19, but not B cell markers, are expressed in neurovascular meta-cells. FIG. 8G is a scatter plot showing the correlation of pericyte marker genes with CD19 expression. Note the separation of the neurovascular cluster. FIG. 8H is a UMAP projection of meta-cells, shaded by mean expression of microglia markers. FIG. 8I is a heatmap showing log 10 average TPM values for selected genes across meta-cells. Dendrograms indicate Ward's hierarchical clustering of each of the two populations shown.

FIGS. 9A-9H illustrate the finding that CD19 is expressed in both pericytes and vSMCs. FIG. 9A illustrates a subset of non-neuronal cells from Zhong, et al. 2018, Nature, 555, 524-528 (hereinafter, Zhong 2018), La Manno et al., 2016, Cell, 167, 566-580 (hereinafter, La Manno 2016), and La Manno et al., 2018, Nature, 560, 494-498 (hereinafter, La Manno 2018). FIG. 9B shows expression of marker genes used for clustering. Max TPM per gene (y-axis) is indicated. FIG. 9C shows (left) low expression of vSMC marker genes and (right) high expression of pericyte marker genes. Y-axis labels indicate maximum TPM value shown. FIG. 9D-9F shows neurovascular and progenitor subset of BICCN data annotated by (FIG. 9D) cluster ID, (FIG. 9E) timepoint, or (FIG. 9F) region. Note that samples were annotated different levels of regional granularity, so resulting annotations are sometimes overlapping. FIG. 9G shows expression of vSMC marker (ACTA2) as well as CSPG4 (pericyte) and CLDN5 (endothelial) markers. CD19 is expressed primarily in the vSMC and pericyte clusters. FIG. 9H is a track plot showing lack of early developmental marker expression and distinguishing markers between vSMC and pericyte clusters. Y-axis labels indicate maximum TPM value shown.

FIGS. 10A-10F illustrates the finding that the CAR-T recognized CD19 isoform is expressed in the adult human brain. FIG. 10A shows expression of CD19 (top) and CD248 (bottom) in data. Prenatal and postnatal samples are shown, and the different samples from distinct regions but the same age are plotted on the same x coordinate. FIG. 10B is a histogram of the distribution of spearman correlation values for all genes with CD19 expression in only postnatal samples. The indicated percentiles indicate the percentile of that gene's correlation. FIG. 10C is a scatter plot of CD248 against CD19 RPKM values in only postnatal samples. FIG. 10D shows enriched GO terms in the top 200 genes by spearman correlation with CD19. FIG. 10E shows gene score distribution in single cells belonging to pericyte or endothelial clusters, as well as other brain cells; along with B cells and other PBMCs. Gene score was calculated with the top 30 genes by spearman correlation. FIG. 10F shows RPKM values per exon of CD19 in the Brainspan data, showing expression of the key exons 2 and 4 for CAR-T cell recognition.

FIGS. 11A-11C illustrate brain pericyte-specific expression of CD19. FIG. 11A is a track plot showing expression of selected marker genes for each population. Note that CD19 expression is limited to brain pericytes, but not lung pericytes. Additionally, brain pericytes express certain transcription factors, such as BCLIIA, that are enriched in B cells. Y-axis labels indicate maximum TPM value. FIG. 11B (top) is a heatmap of log 2 fold change in gene expression of surface/secreted genes between brain and lung pericytes. FIG. 11B (bottom) is a heatmap of expression of the same genes. Abundance data has been quantile-normalized to improve comparison of relative expression between the two populations. FIG. 11C is a series of heatmaps showing (top) log 2 fold-change and (bottom) quantile-normalized abundance, as in FIG. 11B. Comparisons are made between brain pericytes, brain endothelial cells, and B cells (from PBMCs), and genes are ordered by log 2 fold-change between pericytes and B cells.

FIGS. 12A-12D illustrate identification of non-neuronal clusters of interest. FIG. 12A shows UMAR plots showing expression of selected marker genes for mural cells, endothelial cells, and microglia/immune cells (FIGS. 12A-12D). FIG. 12B shows separate clusters of endothelial cells (CD31IPECAM1+) and mural cells (RGS5+) within the central cluster of neurovascular cells. FIG. 12C illustrates lack of ACTA2 expression indicating pericyte identity. FIG. 12D shows CD81 is expressed in mural cells.

FIGS. 13A-13F illustrates meta-cell clustering of BICCN data. FIG. 13A shows an example processing of one sample (GW25 Thalamus). Clustering, doublet identification, as well as expression of selected marker genes is shown. Note that vSMCs, pericytes, and endothelial cells are clustered closely together, but are clearly distinguished by marker gene expression. FIG. 13B shows identification of variable genes across meta-cells. Genes used for downstream analysis are shown in black. FIG. 13C shows the number of cells per meta-cell, grouped by cluster. FIG. 13D shows mean number of genes (left) or counts (right) per cell in each meta-cell, grouped by cluster. FIG. 13E shows mean expression of PAX3, PAX7, and LIN28A per meta-cell, showing the high expression at early timepoints and progressive loss throughout development. FIG. 13F is a set of heatmaps of selected marker genes for each meta-cell in the indicated ranges of sample ages, showing the absence of CD19 and mural cell gene expression at CS12-15 but the presence at GW25.

FIGS. 14A-14F illustrate identification of sub-clusters of interest across brain datasets. FIG. 14A shows integrated clustering of Zhong 2018, La Manno 2016, and La Manno 2018. FIG. 14B shows expression of CD248 (pericyte), CSFIR (microglia), PECAM1 (endothelial), and CD19 in these datasets. FIG. 14C shows CD81 is expressed by CD19+ mural cells. FIG. 14D shows analysis of cells comprising progenitors, vascular cells, and microglia from BICCN data. The cells that were included for subsequent analysis are indicated in the bottom left panel. FIG. 14E shows expression of selected marker genes that were used to identify clusters of cells. FIG. 14F shows expression of vascular SMC marker genes distinguishing venous, arterial, and arteriole SMCs.

FIGS. 15A-15E illustrate analysis of Allen Institute Brainspan data. FIG. 15A is a set of scatter plots showing (top) correlation of CD248 and ANPEP (CD13), two pericyte markers and (bottom) CD248 and CLDN5, an endothelial cell marker gene, showing overall correlation of neurovascular-associated genes. FIG. 15B shows expression of CD19 and CD248 by brain region. FIG. 15C shows integrated clustering of brain and PBMC data, with B cell, pericyte, and endothelial cell clusters identified. FIG. 15D shows comparison of CD19 expression and CD19-correlated gene score, showing high score expression in pericytes and endothelial cells, and smaller enrichment in B cell cluster, indicating that pericytes/vasculature is the primary driver of CD19 expression in the brain. Expression for genes is in counts per 10,000; unit for gene score is arbitrary unit. FIG. 15E shows comparisons of additional genes/correlated gene scores, as controls, showing ability of the approach to selectively enrich for expected populations of cells. Units are as in FIG. 15D.

FIGS. 16A-16E depict analysis of CD19 expression in mouse brain. FIG. 16A shows flow cytometric analysis of CNS neurovascular cells isolated from healthy C57BL/6J mice. The live cell fraction is shown with gating for the CD45+ and CD45− populations. CD19+ cells are present in the CD45− population as shown, along with CD31+ endothelial cells. CD19+ B220+B-cells are found in the CD45+ fraction. FIG. 16B shows fluorescence minus one (FMO) control staining for CD19, with the same gating as in FIG. 16A. FIG. 16C (left) CD19 is expressed at roughly similar levels in the CD45− fraction as in the CD45+ fraction (B-cells); (right) B220 is expressed only by the CD45+CD19+ B-cell fraction, but not in the CD45− CD 19+ fraction. FIG. 16D shows flow cytometry analysis of live, single cells, showing gating for CD45− CD41− CD13+CD31− pericytes, and expression of CD19 against CD13. An isotype control for CD19 is shown. Data in (FIG. 16D) represents a separate experiment from (FIGS. 16A & 16C). FIG. 16E shows scRNA-seq of dissociated mouse brain after enrichment for CD19+, CD31+, and CD19+ cells by FACS, (left) UMAR plot showing identified clusters, (right) expression of marker genes showing low CD19 expression in pericytes and moderate CD19 expression in microglia.

FIGS. 17A-17I depict data from infusion of CD19-directed CAR-T cells into mice. FIG. 17A shows results from flow cytometry based killing assays in human B-ALL cell line Nalm6 and murine CD19+A20 B-ALL cell line in presence of human 4-1BB or CD28 based anti-human or murine CD19 CAR-T cells. FIG. 17B depicts representation of CAR constructs and animal study design using NSG mice. FIG. 17C shows representative immunofluorescence imaging from the cortex showing nuclear staining (DARI) and Evans Blue dye (EBD) extravasation for BBB disruption in NSG mice following murine-targeting CD19 CAR-T treatment (n=5 mice per treatment). Scale bar, 75 pm. FIG. 17D shows quantification of EBD staining in (FIG. 17C) in each group of mice as determined by the product of the mean fluorescence intensity per field area. Representative sections (n=3 per mouse) were analyzed and graphed. *p<0.05, **p<0.005, 2 tailed unpaired t-test. FIG. 17E shows the syngeneic C57BL/6J mouse model recapitulates the murine CD19-specific neurotoxicity pattern observed in the immunodeficient model, showing staining for DARI and EBD in cortical sections (n=5 mice per group). Scale bar, 75 pm. FIG. 17F shows quantification of EBD staining for each group shown in (FIG. 17E). *p<0.05, **p<0.005, 2 tailed unpaired t-test. FIG. 17G shows in vivo imaging using high-resolution MRI with gadodiamide contrast shows BBB leakage after murine-targeting CD19 CAR-T cell therapy. All experiments were performed 4 days after CAR-T cell infusion. Anatomical reference image with selected region of interest (ROI) contour used to calculate delta T1 and T2 between each group is outlined. n=4 mice per group, mean and standard deviation across mice provided. FIG. 17H shows quantification of mean delta T1 of MRI within ROI shown in (FIG. 17G), n=4 mice per group. *p<0.05, two-tailed t-test. FIG. 17I (top) shows decrease in CD45+CD19+ cells (n=5 mice per group) in mCD19-specific CAR-T treated mice as opposed to the hCD19 CART group, (bottom) CD19 vs CD13 (n=5 mice per group) showing decrease in live brain CD45-CD41-CD31-CD13+CD19+ cells in the mCD19 specific CAR-T as opposed to the hCD19 CAR-T treated group. Error bars show the SEM, p-value using two-tailed t-test.

FIGS. 18A-18C illustrate comparison of mural cell transcriptome from brain and lung. FIG. 18A is a scatter plot comparing expression in brain pericytes and lung pericytes. Genes with at least an absolute log 2 fold change of 2 are colored in dark grey, and selected genes are annotated, showing similar expression of markers such as PDGFRB, but distinct expression of other genes such as BCL1 and HOXB4. FIG. 18B as in (FIG. 18A) but comparing lung vSMCs with lung pericytes. FIG. 18C as in (FIG. 18A) but comparing brain endothelial cells and lung endothelial cells.

FIGS. 19A-19D illustrate meningioma (n=4) samples were digested enzymatically and stained with antibodies to CD11b, CD45, CD13, PDGFRb, CD31 and CD19. Isotypes to PDGFRb and CD31 were used. Draq7 was used to remove dead cells. Pericytes were identified as CD13+ PDGFRb+CD31− live CD11b− cells. Numbers in quadrants depict the percentage of CD13+ PDGFRb+CD31− live CD11b− pericytes. For samples Ge512, Ge515 and Ge1405, CD13+ PDGFRb+CD31− live CD11b− cells were sorted as pericytes, whereas CD31+ live CD11b− cells were sorted as endothelial cells. RNA was extracted from sorted cells and used for RNA sequencing analysis to confirm pericyte/endothelial cell subset identification.

FIGS. 20A-20C illustrate meningioma (n=3) samples were digested enzymatically and stained with antibodies to CD11b, CD45, CD13, PDGFRb, CD31 and CD19. Isotypes to PDGFRb and CD31 were used. Draq7 was used to remove dead cells. Pericytes were identified as CD13+ PDGFRb+CD31− live CD11b− cells. Numbers in quadrants depict the percentage of CD13+ PDGFRb+CD31− live CD11b− pericytes. For samples Ge512, Ge515 and Ge1405, CD13+ PDGFRb+CD31− live CD11b− cells were sorted as pericytes, whereas CD31+ live CD11b− cells were sorted as endothelial cells. RNA was extracted from sorted cells and used for RNA sequencing analysis to confirm pericyte/endothelial cell subset identification.

FIGS. 21A-21E: Medulloblatoma (n=1), epilepsy (n=1), GBM (n=1) and schwannoma (n=1) samples were digested enzymatically and stained as described in FIGS. 20A-20C. Pericytes were identified as CD13+ PDGFRb+CD31− live CD11b− cells. Numbers in quadrants depict the percentage of CD13+ PDGFRb+CD31− live CD11b− pericytes. FIG. 21E: Digested epilepsy sample Ge 1373 was cultured for pericyte isolation. After one month, cells were stained with antibodies to CD11b, CD45, CD13, PDGFRb, CD31 and CD19. Isotypes to PDGFRb and CD31 were used. Draq7 was used to remove dead cells. Pericytes were identified as CD13+ PDGFRb+CD31− live CD11b− cells.

FIGS. 22A-22C illustrate identification of pericyte markers in pericytes using single cell transcriptomic datasets (Darmanis et al., (2017) Cell reports, 21(5), 1399-1410). The Vascular cell cluster is enriched in pericyte markers.

FIGS. 23A-23B illustrate identification of pericyte markers (FIG. 23A) and transcription factors controlling CD19 expression (FIG. 23B) in pericytes using single cell transcriptomic datasets. (Developmental timepoint and Brain region GW22T_thalmus from Human_Single Cell 10× RNASeq-Kriegstein).

FIG. 24A: Generation of DNA plasmids of inhibitory CARs to prevent FDA approved CD19 CAR related neurotoxicity (either bispecific activating anti-CD19 and inhibitory anti-pericyte CARs or monospecific inhibitory CARs). FIG. 24B: Generation of gene edited human B-ALL cell lines as control to test candidate CARs: knock in lines (used as positive control) expressing both cell surface antigens (CD19 and pericyte marker CSPG4); as the expression of CSPG4 is low in the raj cell line, current work includes generation of a novel expression vector where both GFP and CSPG4 expression are under the control of a single potent human promoter EFlalpha.

FIG. 25 illustrates an AND NOT logic gated bi-specific CAR.

### DETAILED DESCRIPTION

The present invention is based on the unexpected finding that CD19 is expressed on mural cells, including neurovascular pericytes and/or vascular smooth muscle cells (vSMCs) in the brain. Provided herein are methods for treating cancer utilizing modified T cells that express a CD19 chimeric antigen receptor (CAR) and an inhibitory CAR (iCAR) capable of binding a CD19-expressing mural cell (e.g. pericyte or vSMC). Such methods provide a reduction in CAR T cell-induced neurotoxicity.

Mural cells are an integral part of the neurovascular unit (NVU), which surround endothelial cells and are critical for regulating the integrity of the BBB. Mural cells include pericytes and vSMCs, which are closely related cell types that differ anatomically: pericytes localize along capillaries, while vSMCs are found along larger vessels, including arteries, arterioles, and venules. These cells are transcriptionallyl similar, sharing the identity of any marker genes and appearing to exist on a transcriptional lineage continuum. ACTA2, encoding alpha-smooth muscle actin, is a canonical marker used to distinguish these two populations, which is significantly upregulated in vSMCs. Many pericyte markers, however, such as CSPG4 and RGS5, are also highly expressed in vSMCs, causing brain vSMCs to often be annotated as pericytes.

The invention also includes an inhibitory CAR specific for a mural cell (e.g. pericyte or vSMC), compositions comprising the CAR, polynucleotides encoding the CAR, vectors comprising a polynucleotide encoding the CAR, and recombinant T cells comprising the CAR. Also provided herein are methods of making a modified T cell expressing a mural cell-specific inhibitory CAR.

### A. Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in some instances ±5%, in some instances ±1%, and in some instance ±0.1% from the specified value, as such variations are appropriate to perform the disclosed methods.

The term “antibody,” as used herein, refers to an immunoglobulin molecule capable of binding to an antigen. Antibodies can be intact immunoglobulins derived from natural sources or from recombinant sources and can be antigen-binding portions of intact immunoglobulins. Antibodies are typically tetramers of immunoglobulin molecules. The antibody may exist in a variety of forms where the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, “Fab” refers to a fragment of an antibody structure that binds to an antigen but is monovalent and does not have an Fc portion, for example, an antibody digested by the enzyme papain yields two Fab fragments and an Fc fragment (e.g., a heavy (H) chain constant region designated as an Fc fragment that does not bind antigen). As used herein, “F(ab′)2” refers to an antibody fragment generated by pepsin digestion of whole IgG antibodies having two antigen-binding (ab′) (bivalent) regions, each (ab′) region comprising two separate amino acid chains, a part of a heavy (H) chain and a light (L) chain linked by a disulfide (SS) bond which maintains the structure of the binding pocket for binding an antigen and where the remaining H chain portions are linked together. A “F(ab′)2” fragment can be split into two individual Fab′ fragments.

The term “high affinity” as used herein refers to a strong interaction of one molecule with a target molecule.

As used herein, the term “autologous” refers to any material derived from the same individual to which it is later to be re-introduced or administered.

As used herein, the term “allogeneic” refers to any material derived from a different individual of the same species than the individual to which is later re-introduced or administered.

The term “chimeric antigen receptor” or “CAR”, as used herein, refers to an engineered receptor that is expressed on a T cell or any other effector cell type capable of cell-mediated cytotoxicity. The CAR includes an antigen-binding domain or fragment (e.g., an extracellular domain) thereof that is specific for a ligand or receptor. The CAR optionally also includes a transmembrane domain, an intracellular domain and a signaling domain.

The term “inhibitory CAR” or “iCAR” refers to a chimeric antigen receptor (CAR) comprising an antigen-binding domain and an inhibitory signaling domain. When the iCAR is expressed on the surface of an immune cell (e.g. a T cell) and binds to its cognate antigen, an inhibitory signal is transduced through the cell via the inhibitory signaling domain of the iCAR, thereby turning off the effector functions of the immune cell (e.g. cytotoxic activity of a T cell).

“Co-stimulatory ligand,” as the term is used herein, includes a molecule on an antigen presenting cell (e.g., an artificial antigen presenting cell or “aAPC”, dendritic cell, B cell, and the like) that specifically binds a cognate co-stimulatory receptor on a T cell, thereby providing a signal which, in addition to the primary signal provided by, for instance, binding of a TCR/CD3 complex to an MHC molecule loaded with peptide, mediates a T cell response, including, but not limited to, proliferation, activation, differentiation, and the like.

A “co-stimulatory molecule” or “co-stimulatory receptor” refers to the cognate binding partner on a T cell that specifically binds with a co-stimulatory ligand, thereby mediating a co-stimulatory response by the T cell, such as, but not limited to, proliferation, activation, differentiation, and the like. Co-stimulatory molecules include, but are not limited to CD27, CD28, CD40, or 4-1BB.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Effective amount” or “therapeutically effective amount” are used interchangeably herein, and refer to an amount of a compound, formulation, material, or composition, as described herein effective to achieve a particular biological result. Such results may include, but are not limited to, the inhibition of CD19 targeted therapy cytotoxicity against neurovascular mural cells (e.g. pericytes or vSMCs), as determined by any means suitable in the art.

The term “effector function” refers to a specialized function of an immune cell.

As used herein, the term “endogenous” refers to substances or processes that originate from within a particular system, such as an organism, tissue, or cell. In contrast, the term “exogenous” as used herein refers to substances or processes that originate from outside a particular system, such as an organism, tissue, or cell. Thus, for example, a T cell expressing a CAR has been engineered to express an exogenous fusion protein.

The term “expression” as used herein is defined as the transcription and/or translation of a particular nucleotide sequence driven by a promoter.

“Expression vector” refers to a vector comprising one or more recombinant polynucleotide sequences comprising expression control elements operatively linked to a nucleotide sequence to be expressed. An expression vector comprises sufficient cis-acting elements for gene expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “inhibition” refers to a primary response induced by binding of an inhibitory molecule (e.g., PD-1) with its cognate ligand, thereby mediating a signal transduction event that attenuates, terminates, or opposes activation signals in a T cell. Inhibition can mediate altered T cell activation by, for example, attenuating, opposing, or disrupting MHC/TCR signaling and/or signaling through costimulatory molecules, inducing anergy/exhaustion, inducing the death of the T cell via activation of apoptosis, and the like.

The term “inhibitory receptor” as used herein, means a molecule that specifically binds with a cognate inhibitory ligand and inhibits or attenuates the primary response by the cell, including, but not limited to inhibiting activation, proliferation, cytotoxic function, cytokine production/secretion, and the like. Inhibitory molecules are well-known in the art and include but are not limited to PD1, CTLA-4, LAG3, TIM3, 2B4, and BTLA.

The term “intracellular signaling domain” is meant to include any truncated portion of the intracellular domain sufficient to transduce the effector function signal.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “limited toxicity” as used herein, refers to the peptides, polynucleotides, cells and/or antibodies of the invention manifesting a lack of negative biological effects, anti-tumor effects, or negative physiological symptoms toward a healthy cell, non-tumor cell, non-diseased cell, non-target cell or population of such cells either in vitro or in vivo.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, “mural cells” include vessel-associated cell types such as pericytes and vascular smooth muscles cells (vSMC).

Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

“Pericytes” are mural cells surrounding blood vessels, adjacent to endothelial cells. Pericytes play critical roles in maturation and maintenance of vascular branching morphogenesis. In the central nervous system (CNS), pericytes are necessary for the formation and regulation of the blood-brain barrier (BBB) and pericyte deficiency accompanies CNS diseases including multiple sclerosis, diabetic retinopathy, neonatal intraventricular hemorrhage, and neurodegenerative disorders.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. In some embodiments, a nucleic acid sequence is considered to have at least 95%, 96%, 97%, 98%, or 99% identity or homology to any nucleic acid sequence disclosed herein.

As used herein, the terms “peptide,” “polypeptide,” and “protein” are used interchangeably to refer to a polymer of amino acid residues covalently linked by peptide bonds. A protein or peptide must contain at least two amino acids, and no limitation is placed on the maximum number of amino acids that can comprise a protein or peptide sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. In some embodiments, an amino acid sequence is considered to have at 95%, 96%, 97%, 98%, or 99% identity or homology to any amino acid sequence described herein.

The term “proinflammatory cytokine” refers to a cytokine or factor that promotes inflammation or inflammatory responses. Exemplary proinflammatory cytokines include, but are not limited to, chemokines (CCL, CXCL, CX3CL, XCL), interleukins (such as, IL-1, IL-2, IL-3, IL-5, IL-6, IL-7, IL-9, IL10 and IL-15), interferons (IFNγ), and tumor necrosis factors (TNFα and TNFβ).

The term “promoter” refers to a regulatory region of DNA located near a gene, providing a control point for regulated gene transcription. A promoter comprises specific DNA sequences recognized by proteins known as transcription factors that bind to the promoter sequence and recruit RNA polymerase, the enzyme that performs template-directed synthesis of RNA from the coding strand of the DNA.

A “tissue-specific” promoter is a nucleotide sequence which, when operably linked to a polynucleotide encoding a gene product, causes the gene product to be produced in a cell substantially only if the cell is a cell of the tissue type corresponding to the promoter.

A “signal transduction pathway” refers to the series of steps required to transmit a signal from one component of or location in a biological system to another. For example, the steps involved in transmitting a signal triggered by the binding of a T cell receptor to its cognate peptide presented in the context of an HLA allele on the surface of an antigen-presenting cell. The phrase “cell surface receptor” includes molecules and complexes of molecules capable of receiving a signal and transmitting signal across the membrane of a cell.

By the term “specifically binds,” as used herein, refers to an antibody, a receptor, or a ligand capable of recognizing and binding to a cognate binding partner (e.g., a stimulatory, costimulatory, or inhibitory molecule present on a T cell) present in a sample, but which antibody or ligand does not substantially recognize or bind other molecules in the sample.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

The term “subject” as used herein refers to any living organism in which an adaptive immune response can be elicited (e.g., mammals). Preferably, the subject is a human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cells that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a host cell. A “transfected” or “transformed” or “transduced” cell is one which has been transfected, transformed or transduced with one or more exogenous nucleic acid molecules. The term “cell” in this context includes the primary subject cell and its progeny.

“Transmembrane domain” refers to a portion or a region of a molecule that spans a lipid bilayer membrane.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Inhibitory CARs (iCARs)

In one aspect, the invention provides an inhibitory CAR (iCAR) that is capable of specifically binding a mural cell (e.g. a pericyte or vSMC) and preventing T cell-mediated killing of the mural cell (e.g. pericyte or vSMC). The iCAR comprises an antigen binding domain that is capable of binding a mural cell (e.g. pericyte or vSMC), an inhibitory signaling domain, and optionally, a transmembrane domain.

### Antigen-Binding Domain

The antigen binding domain of an iCAR is an extracellular region of the iCAR for binding to a specific target antigen including, but not limited to, a protein, carbohydrate, or glycolipid. In some embodiments, the iCAR comprises an antigen-binding domain having affinity for a target antigen with expression restricted to one or more specific cell types (e.g., mural cells, e.g pericytes or vSMCs). In some embodiments, the iCAR comprises affinity to a target antigen on a target cell, (e.g. a mural cell, e.g a pericyte or vSMC). The target antigen may include any type of protein, or epitope thereof, associated with the target cell. In some embodiments, the antigen-binding domain has affinity for a target antigen restricted to cells present at the blood-brain barrier (BBB). In some embodiments, the cells present at the blood-brain barrier (BBB) are mural cells, and the target antigen comprises a mural cell-associated or mural cell-specific antigen. In some embodiments, the cells present at the blood-brain barrier (BBB) are pericytes, and the target antigen comprises a pericyte-associated or pericyte-specific antigen. In some embodiments, the cells present at the blood-brain barrier (BBB) are vSMC, and the target antigen comprises a vSMC-associated or vSMC-specific antigen. In some embodiments, the target antigen is a mural cell-associated antigen. In some embodiments, the target antigen is a mural cell-specific antigen. In some embodiments, the target antigen is a pericyte-associated antigen. In some embodiments, the target antigen is a pericyte-specific antigen. In some embodiments, the target antigen is a vSMC-specific antigen. In some embodiments, the target antigen is a vSMC-associated antigen. In some embodiments, the mural cell-associated antigen (e.g. pericyte-associated antigen or vSMC-associated antigen) is selected from the group consisting of cluster of differentiation 146 (CD146 or MUC18 or MCAM), platelet-derived growth factor receptor beta (PDGFRB), regulator of G protein signaling 5 (RGS5), cluster of differentiation 248 (CD248 or endosialin), chondroitin sulfate proteoglycan 4 (NG2 or CSPG4), biglycan (BGN), fibronectin 1 (FN1), semaphorin 5A (SEMA5A), plexin domain containing 1 (PLXDC1), thy 1 surface antigen (THY1), cadherin (CDH6), tissue factor pathway inhibitor (TFPI), collagen type 1α2 chain (COL1A2), integrin subunit α1 (ITGA1), endothelin receptor type A (EDNRA), protocadherin 18 (PCDH18), cadherin 11 (CDH11), AXL receptor tyrosine kinase (AXL), neurotrimin (NTM), Tumor Necrosis Factor receptor superfamily member 1A (TNFRSF1A), sphingosine-1-phosphate receptor (S1PR3), coagulation factor III, and tissue factor (F3). In certain embodiments of the invention, the iCAR is specific for an antigen expressed on mural cells (e.g. pericyte or vSMC) but not expressed on the tumor cells to be treated.

CD146, also known as the melanoma cell adhesion molecule (MCAM) or cell surface glycoprotein MUC18, is a 113 kDa cell adhesion molecule currently used as a marker for cells of the endothelial cell lineage. In humans, the CD146 protein is encoded by the MCAMgene.

Platelet-derived growth factor receptor beta (PDGFRB) is a member of the receptor tyrosine kinase family. Activation of PDGFRB leads to activation of downstream signaling pathways, inducing cellular proliferation, differentiation, survival, and migration.

Regulator of G protein signaling 5 (RGS5) is a signal transduction molecule that is involved in the regulation of heterotrimeric G proteins by acting as GTPase activator.

CD248 (endosialin) is a transmembrane glycoprotein that is dynamically expressed on pericytes and fibroblasts during tissue development, tumor neovascularization and inflammation.

CD248 is a member of the “Group XIV” family of C-type lectin transmembrane receptors which play roles in cell-cell adhesion and in host defense.

CSPG4 is a proteoglycan which plays a role in cell proliferation and migration which stimulates endothelial cells motility during microvascular morphogenesis.

Thus, the antigen-binding domain of the iCAR is capable of binding to, and has affinity for, mural cell (e.g. pericyte or vSMC)-associated antigens. In some embodiments, the mural cell-associated antigen is mural cell-specific. In some embodiments, the pericyte-associated antigen is pericyte-specific. In certain embodiments, the antigen-binding domain has affinity for a surface antigen expressed on the surface of a mural cell. In certain embodiments, the antigen-binding domain has affinity for a surface antigen expressed on the surface of a mural cell (e.g. pericyte or vSMC). In certain embodiments, the antigen-binding domain has affinity for, and is capable of binding to, an antigen selected from the group consisting of CD146, PDGFRB, RGS5, CD248, and NG2/CSPG4.

The antigen-binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen-binding domain portion comprises a mammalian antibody or a fragment thereof. The choice of antigen-binding domain may depend upon the type and number of antigens that are present on the surface of a target cell. The antigen-binding domain may comprise a commercial antibody or a fragment thereof that binds to a target antigen. Exemplary antibodies with binding affinity for mural cell (e.g. pericyte or vSMC)-associated antigens, include, but are not limited to, monoclonal antibody, monoclonal antibody P1H12 to human CD146 (Abcam: ab24577), monoclonal antibody Y92 to human PDGFR beta-C-terminal (Abcam: ab32570), monoclonal antibody CL5568 to RGS5 (Abcam: ab243029), and monoclonal antibody OTI1H6 to CD248 (Invitrogen: MA5-26838), monoclonal antibody D120.43/D4.11/N143.8/N109.6 to NG2 (Invitrogen: 37-2700).

In some embodiments, the antigen-binding domain is selected from the group consisting of an antibody, an antigen-binding fragment (Fab), and a single-chain variable fragment (scFv). In some embodiments, the antigen-binding domain of the present invention is selected from the group consisting of a CD146-specific antibody, a CD146-specific Fab, and a CD146-specific scFv. In some embodiments, the antigen-binding domain of the present invention is selected from the group consisting of a PDGFRB-specific antibody, a PDGFRB-specific Fab, and a PDGFRB-specific scFv. In some embodiments, the antigen-binding domain of the present invention is selected from the group consisting of a CD248-specific antibody, a CD248-specific Fab, and a CD248-specific scFv. In some embodiments, the antigen-binding domain of the present invention is selected from the group consisting of a RGS5-specific antibody, a RGS5-specific Fab, and a RGS5-specific scFv. In some embodiments, the antigen-binding domain of the present invention is selected from the group consisting of a CSPG4-specific antibody, a CSPG4-specific Fab, and a CSPG4-specific scFv.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen-binding domain (e.g., CD248 binding domain) comprises an scFv having the configuration from N-terminus to C-terminus, VH-linker-VL. In some embodiments, the antigen-binding domain comprises an scFv having the configuration from N-terminus to C-terminus, VL-linker-VH. Those of skill in the art will be able to select the appropriate configuration for use in the present invention.

The linker is usually rich in glycine for flexibility, and rich in serine or threonine for solubility. The linker generally joins the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO:1), (GGGS)ₙ (SEQ ID NO:2), and (GGGGS)ₙ (SEQ ID NO:3), where n represents an integer of at least 1. Exemplary linker sequences can comprise amino acid sequences including, without limitation, GGSG (SEQ ID NO:4), GGSGG (SEQ ID NO:5), GSGSG (SEQ ID NO:6), GSGGG (SEQ ID NO:7), GGGSG (SEQ ID NO:8), GSSSG (SEQ ID NO:9), GGGGS (SEQ ID NO:10), GGGGSGGGGSGGGGS (SEQ ID NO:11) and the like. Those of skill in the art will be able to select the appropriate linker sequence for use in the present invention. In one embodiment, an antigen-binding domain of the present invention comprises a heavy chain variable region (VH) and a light chain variable region (VL), wherein the VH and VL are separated by a linker sequence having the amino acid sequence GGGGSGGGGSGGGGS (SEQ ID NO:11), which may be encoded by the nucleic acid sequence GGTGGCGGTGGCTCGGGCGGTGGTGGGTCGGGTGGCGGCGGATCT (SEQ ID NO:12).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

In some embodiments, the antigen-binding domain may be derived from the same species in which the iCAR will ultimately be used. For example, for use in humans, the antigen-binding domain of the iCAR may comprise a human antibody or a fragment thereof. In some embodiments, the antigen-binding domain may be derived from a different species in which the iCAR will ultimately be used. For example, for use in humans, the antigen-binding domain of the iCAR may comprise a murine antibody or a fragment thereof.

In certain embodiments, the antigen-binding domain is a ligand capable of binding a receptor on the surface of a mural cell (e.g. pericyte or vSMC).

In certain embodiments, an iCAR of the present disclosure may have affinity for more than one target antigen on one or more target cell type. In such embodiments, the iCAR is a bispecific iCAR, or a multispecific iCAR. In certain embodiments, the iCAR comprises one or more target-specific antigen-binding domains that confer affinity for one or more target antigens. For example, an exemplary bispecific iCAR may have a first binding domain that confers affinity for a first mural cell (e.g. pericyte or vSMC)-associated antigen (e.g. CD248) and a second binding domain that confers affinity for a second mural cell (e.g. pericyte or vSMC)-associated antigen (e.g. RGS5). In certain embodiments, the iCAR may comprise one or more antigen-binding domains that confer affinity for the same target antigen. For example, an iCAR comprising one or more antigen-binding domains having affinity for the same target antigen could bind distinct epitopes of that target antigen.

When a plurality of antigen-binding domains is present in an iCAR, the binding domains may be arranged in tandem and may be separated by linker peptides. For example, in an iCAR comprising two antigen-binding domains, the binding domains are connected to each other covalently on a single polypeptide chain, through an oligo- or polypeptide linker, an Fc hinge domain, or a membrane hinge domain.

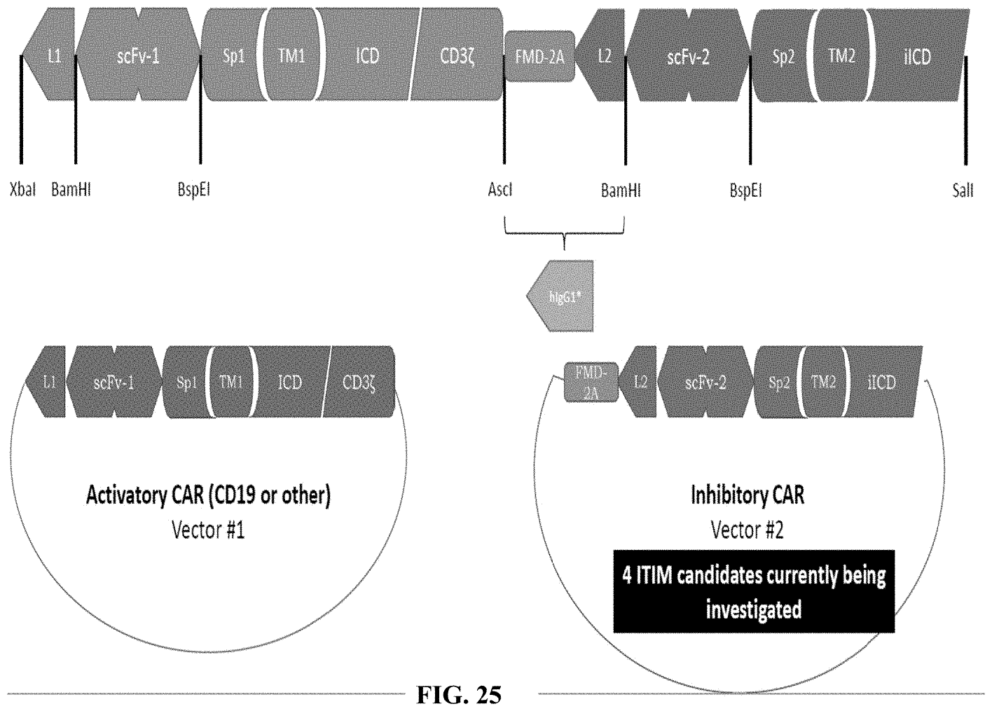

In certain aspects, the invention includes a bi-specific iCAR comprising an activatory anti-CD19 CAR and an inhibitory anti-mural cell CAR (e.g. anti-pericyte or anti-vSMC) (see e.g FIG. 25). In certain embodiments, the activatory CAR comprises an antigen-binding domain capable of binding CD19, a transmembrane domain, an intracellular domain, and a CD3zeta domain and the iCAR comprises an antigen-binding domain capable of binding a mural cell (e.g. pericyte or vSMC), a transmembrane domain, and an intracellular signaling domain. In certain embodiments, the intracellular signaling domain of the inhibitory CAR comprises PTPN6, LAIR1, PD-1, and/or KIR2DL4.

### Inhibitory Signaling Domain

The iCAR comprises an intracellular domain that provides an inhibitory signal to the cell upon engagement of the iCAR with its target. The inhibitory signal serves to inhibit or suppress the activity of the cell from which it is expressed (e.g. a modified T cell). Due to this inhibitory potential, iCARs are distinct and distinguishable from CARs, which are receptors that activate a T cell response. For example, CARs are activating receptors as they typically include CD3zeta, whereas iCARs do not contain activating domains.

The inhibitory signaling domain may comprise any molecule or fragment thereof that serves to send an inhibitory signal to the cell. Such molecules include but are not limited to PD-1, CTLA-4, ICOS, LAG-3, 2B4, BTLA, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, PTPN6, LAIR1, KR2DL4 and any fragments or combination thereof. In certain embodiments, the inhibitory signaling domain comprises the intracellular domain of an inhibitory signaling molecule, or an active fragment thereof capable of transducing a signal. In certain embodiments, the inhibitory signaling domain is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP PTPN6, LAIR1, KIR2DL4 and any combination thereof. Any of the inhibitory signaling domains disclosed herein may be combined with any of the antigen-binding domains disclosed herein to make up the iCAR.

### Transmembrane Domain

iCARs of the present invention may comprise a transmembrane domain disposed between the antigen-binding domain of the iCAR and the inhibitory signaling domain of the iCAR. The transmembrane domain of an iCAR is a domain or amino acid sequence capable of spanning the plasma membrane of a cell (e.g., an engineered immune cell), e.g., a eukaryotic cell.

In some embodiments, the transmembrane domain is derived from a naturally-occurring membrane protein, for example, a Type I transmembrane protein. In some embodiments, the transmembrane domain is an artificial or synthetic sequence that facilitates insertion of the iCAR into a cell membrane, for example, an artificial hydrophobic sequence of appropriate length. Exemplary transmembrane domains include, without limitation, transmembrane domains derived from protein fragments comprising at least the transmembrane region of PD-1, CTLA-4, ICOS, LAG-3, TIM3, 2B4, and BTLA.

Further exemplary transmembrane domains include, but are not limited to, the alpha, beta, gamma, or delta polypeptide subunits of the T cell receptor, CD2, CD28, CD3 epsilon, CD3 zeta, CD45, CD4, CD5, CD7, CD8, CD9, CD16, CD22, CD33, CD37, CD64, CD80, CD86, CD134 (OX-40), CD137 (4-1B), CD154 (CD40L), Toll-like receptor 1 (TLR1), TLR2, TLR3, TLR4, TLR5, TLR6, TLR7, TLR8, and TLR9. In some embodiments, the transmembrane domain may be a synthetic peptide comprising predominantly hydrophobic residues such as leucine and valine, preferably having a triplet of phenylalanine, tryptophan and valine at each end.

The transmembrane domains described herein can be combined with any of the antigen-binding domains described herein, any of the inhibitory signaling domains described herein, or any of the other domains described herein to produce the iCAR.

In some embodiments, the iCAR further comprises a hinge domain. In some embodiments, the hinge domain of an iCAR is disposed between the antigen-binding domain and the transmembrane domain. The hinge domain is an optional component for the CAR. The hinge domain may comprise a domain selected from the group consisting of Fc fragments of antibodies, hinge domains of antibodies, CH2 regions of antibodies, CH3 regions of antibodies, artificial hinge sequences or combinations thereof. Exemplary hinge domains include, without limitation, a CD8a hinge, CH1 and CH3 domains of IgGs (such as human IgG4), and artificial hinges comprising peptides as small as three glycine residues (Gly).

In some embodiments, the iCAR comprises a hinge domain that connects the antigen-binding domain with the transmembrane domain (TM), which, in turn, connects to the inhibitory signaling domain (ISD), such that the iCAR comprises, from amino- to carboxy-terminus: scFv-Hinge-TM-ISD. The hinge domain is a flexible domain, thus allowing the antigen-binding domain to have a structure to optimally recognize the specific structure and density of the target antigens on a cell such as tumor cell (Hudecek et al., supra). The flexibility of the hinge domain permits the hinge domain to adopt many different conformations.

In some embodiments, the hinge domain is an immunoglobulin heavy chain hinge domain. In some embodiments, the hinge domain is a hinge domain polypeptide derived from a receptor (e.g., a CD8-derived hinge domain). In some embodiments, the hinge domain is derived from any one of the following inhibitory receptors: PD-1, CTLA-4, ICOS, LAG-3, 2B4, and BTLA.

The hinge domain can have a length of from about 4 amino acids (aa) to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge domain can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge domains can have any of a number of suitable lengths, such as from 1 amino acid to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge domains can have a length of greater than 20 amino acids (e.g., 30, 40, 50, 60 or more amino acids).

Non-limiting exemplary synthetic hinge domains include, for example, glycine polymers (G)ₙ, glycine-serine polymers (including, for example, (GS)ₙ, (GSGGS)ₙ (SEQ ID NO:1) and (GGGS)ₙ (SEQ ID NO:2), where n is an integer of at least one), glycine-alanine polymers, alanine-serine polymers, and other flexible linkers known in the Other exemplary hinge domains can comprise amino acid sequences include, without limitation, GGSG (SEQ ID NO:4), GGSGG (SEQ ID NO:5), GSGSG (SEQ ID NO:6), GSGGG (SEQ ID NO:7), GGGSG (SEQ ID NO:8), GSSSG (SEQ ID NO:9), and the like.

In some embodiments, the hinge domain comprises an immunoglobulin heavy chain hinge domain. Immunoglobulin hinge domain amino acid sequences are known in the art; see, e.g., Tan et al., Proc. Nat. Acad. Sci. USA (1990) 87(1):162-166; and Huck et al., Nucleic Acids Res. (1986) 14(4): 1779-1789. As non-limiting examples, an immunoglobulin hinge domain can include one of the following amino acid sequences: DKTHT (SEQ ID NO:13); CPPC (SEQ ID NO:14); CPEPKSCDTPPPCPR (SEQ ID NO:15) (see, e.g., Glaser et al., J. Biol. Chem. (2005) 280:41494-41503); ELKTPLGDTTHT (SEQ ID NO:16); KSCDKTHTCP (SEQ ID NO:17); KCCVDCP (SEQ ID NO:18); KYGPPCP (SEQ ID NO:19); EPKSCDKTHTCPPCP (SEQ ID NO:20) (human IgG1 hinge); ERKCCVECPPCP (SEQ ID NO:21) (human IgG2 hinge); ELKTPLGDTTHTCPRCP (SEQ ID NO:22) (human IgG3 hinge); SPNMVPHAHHAQ (SEQ ID NO:23) (human IgG4 hinge); and the like.

In some embodiments, the hinge domain comprises one or more conservative amino acid substitutions and/or insertions and/or deletions compared to a wild-type (naturally-occurring) hinge domain. For example, His229 of human IgG1 hinge can be substituted with Tyr, so that the hinge domain comprises the sequence EPKSCDKTYTCPPCP (SEQ ID NO:24); see, e.g., Yan et al., J. Biol. Chem. (2012) 287: 5891-5897. In some embodiments, the hinge domain comprises an amino acid sequence derived from human CD8, or a variant thereof.

### C. Modified Cells

The present invention provides a modified immune cell or precursor thereof (e.g., a T cell). In some embodiments, the modified immune cell comprises an inhibitory CAR (iCAR) comprising an antigen-binding domain capable of binding a mural cell-associated or mural cell-specific antigen (e.g. pericyte-associated or pericyte-specific antigen, or vSMC-associated or vSMC-specific antigen) and an inhibitory signaling domain. In some embodiments, the cell comprises a chimeric antigen receptor (CAR) and an inhibitory CAR (iCAR). In some embodiments, the CAR comprises an antigen-binding domain capable of binding CD19, a transmembrane domain, and an intracellular signaling domain, and the iCAR comprises an antigen-binding domain capable of binding a mural cell-associated or mural cell-specific antigen (e.g. pericyte-associated or pericyte-specific antigen or vSMC-associated or vSMC-specific antigen) and an inhibitory signaling domain.

In some embodiments, the modified immune cell or precursor cell thereof comprises a nucleic acid encoding an iCAR comprising an antigen-binding domain capable of binding a mural cell-associated or mural cell-specific antigen (e.g. pericyte-associated or pericyte-specific antigen or vSMC-associated or vSMC-specific antigen) and an inhibitory signaling domain. In some embodiments, the modified immune cell or precursor cell thereof comprises a nucleic acid encoding an exogenous CAR comprising an antigen-binding domain capable of binding CD19, a transmembrane domain, and an intracellular signaling domain and a nucleic acid encoding an exogenous iCAR comprising antigen-binding domain capable of binding a mural cell-associated or mural cell-specific antigen (e.g. pericyte-associated or pericyte-specific antigen or vSMC-associated or vSMC-specific antigen) and an inhibitory signaling domain.

In some embodiments, the CD19 CAR comprises an antigen-binding domain that has affinity for, and is capable of binding to, CD19 on a target cell (e.g. a tumor cell and/or a mural cell e.g. pericyte or vSMC). In some embodiments, the antigen-binding domain comprises any domain that specifically binds to CD19. In some embodiments, the domain that specifically binds to CD19 comprises a monoclonal antibody, a polyclonal antibody, a synthetic antibody, a human antibody, a humanized antibody, a non-human antibody, and any fragment thereof, including a Fab or scFv. In some embodiments, the CD19 CAR further comprises a transmembrane domain. In some embodiments, the transmembrane domain comprises a domain derived from (i.e. comprise at least the transmembrane region(s) of) the alpha, beta, delta, or gamma polypeptide subunits of the T cell receptor, CD28, CD3 epsilon, CD3 zeta, CD45, CD4, CD5, CD7, CD8, CD9, CD16, CD22, CD33, CD37, CD64, CD80, CD86, CD134 (OX-40), CD137 (4-1B), CD154 (CD40L), Toll-like receptor 1 (TLR1), TLR2, TLR3, TLR4, TLR5, TLR6, TLR7, TLR8, and TLR9. The transmembrane domain may optionally comprise a hinge domain. The transmembrane domain of the CD19 CAR may include any of the hinge domains that are discussed in detail elsewhere herein.

In some embodiments, the CD19 CAR further comprises an intracellular signaling domain. The intracellular signaling domain of the CAR transduces an activation signal for at least one of the effector functions of the immune cell expressing the CAR (e.g., a T cell) and directs the cell (e.g., a T cell) to perform its specialized function, e.g., mediating antigen-specific destruction of a target cell. Non-limiting exemplary intracellular signaling domains include signaling domains or fragments thereof derived from one or more of the following proteins, CD3zeta, CD3gamma, CD3delta, CD3epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof. In some embodiments, the CD19 CAR comprises an intracellular signaling domain that is not CD28.

In certain embodiments, the CD19 CAR comprises a CD19-specific scFV, a transmembrane domain, a 4-1BB intracellular domain and a CD3zeta intracellular domain. In certain embodiments, the CD19 CAR comprises a CD19 specific scFV, a transmembrane domain, an ICOS intracellular domain and a CD3zeta intracellular domain.

In certain embodiments, the modified cells are T cells.

In certain embodiments, the modified cell is the progeny of the original cell that had the modification (e.g. the modified immune cell does not have to have the actual molecular manipulation, but can be the offspring of the immune cell that was modified and had divided into daughter cells).

### D. Methods of Treatment

The present invention is based on the unexpected finding that some mural cells (e.g. pericytes or vSMCs) at the blood-brain barrier (BBB) express CD19.

In one aspect, provided herein are methods of treating cancer in a subject in need thereof. In some embodiments, the methods comprise administering to the subject a modified T cell comprising a CD19 CAR and an inhibitory CAR. In some embodiments, the CD19 CAR comprises an antigen-binding domain capable of binding CD19, a transmembrane domain, and an intracellular signaling domain, and the iCAR comprises an antigen-binding domain capable of binding a mural cell-associated or mural cell-specific antigen (e.g. pericyte-associated antigen or a pericyte-specific antigen, or vSMC-associated or vSMC-specific antigen) and an inhibitory signaling domain.

After the cells comprising a CD19-specific CAR and an iCAR are administered to the subject, on encountering a CD19-expressing cell, whether a healthy mural cell (e.g. pericyte or vSMC) present at the BBB or a cancerous B cell, the CD19 CAR will bind to CD19, transducing an activation signal turning on the cytotoxic activity of the CAR T cell. When the CD19-specific CAR binds to CD19 expressed on a mural cell (e.g. pericyte or vSMC) at the BBB, however, the iCAR will bind a mural cell-associated or mural cell-specific antigen (e.g. pericyte-associated or pericyte-specific antigen, or vSMC-associated or vSMC-specific antigen) expressed on the surface of the mural cell (e.g. pericyte or vSMC) and transduce an inhibitory signal via the inhibitory signaling domain, thereby turning off the cytotoxic activity of the CD19-specific CAR, thereby sparing the CD19-expressing mural cell (e.g. pericyte or vSMC) from destruction. Thus, CAR T cell treatment remains effective against the cancer but does not induce neurotoxicity.

The modified immune cells (e.g., T cells comprising a CAR and/or iCAR) described herein may be formulated as a pharmaceutical composition for delivery to a subject as an adoptive cell therapy. The pharmaceutical composition may further comprise a pharmaceutically acceptable carrier. A therapeutically effective amount of the pharmaceutical composition comprising the modified immune cells may be administered to a human subject in need thereof.

In one aspect, provided herein are methods for adoptive cell therapy comprising administering to a subject in need thereof a composition (e.g., a pharmaceutical composition) comprising modified immune cells (e.g., T cells) as further described herein. In another aspect, provided herein are methods of treating a disease or condition in a subject comprising administering to a subject in need thereof a population of modified immune cells (e.g., T cells).

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive cell therapy, comprises an autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, comprises an allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells of the present invention can be administered to an animal, preferably a mammal, even more preferably a human, to treat a cancer. In addition, the cells of the present invention can be used for the treatment of any condition related to a cancer or the cancer treatment itself, for example a cell-mediated immune response against mural cells (e.g. pericytes or vSMCs) at the blood brain barrier, or neurotoxicity induced by CART therapy. The types of cancers to be treated with the modified cells or pharmaceutical compositions of the invention include, certain leukemia or lymphoid malignancies, benign and malignant tumors, and malignancies that express CD19. Other exemplary cancers include but are not limited prostate cancer, melanoma, B-cell lymphomas and leukemias, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a leukemia. In one embodiment the cancer is a solid tumor.

The administration of the cells of the invention may be carried out in any convenient manner known to those of skill in the art. The cells of the present invention may be administered to a subject by injection, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured by known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day).

In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade >3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In some embodiments, the degree or extent of persistence of administered cells can be detected or quantified after administration to a subject. For example, in some aspects, quantitative PCR (qPCR) is used to assess the quantity of cells expressing the exogenous receptor (e.g., CAR and/or iCAR) in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the exogenous receptor (e.g. exogenous CAR and/or iCAR) can be used to distinguish the administered cells from endogenous cells in a subject.

In one aspect, the invention includes a method of treating cancer in a subject in need thereof comprising administering to the subject a modified T cell comprising a CAR and an iCAR. The CAR comprises an antigen-binding domain capable of binding CD19, a transmembrane domain, and an intracellular domain, and the iCAR comprises an antigen-binding domain capable of binding a mural cell (e.g. pericyte or vSMC (but not CD19), and an inhibitory signaling domain. In certain embodiments, the administering results in reduced neurotoxicity in the subject as compared to neurotoxicity that results from administering an analogous CAR T lacking a mural cell-specific (e.g. pericyte-specific or vSMC-specific) iCAR.

Certain aspects of the invention provide methods for avoiding neurotoxicity due to treatment, in part or in whole. The neurotoxicity may be induced by CAR T cell treatment. In one aspect, the invention includes a method of inhibiting CAR T cell-induced neurotoxicity comprising administering to the subject an effective amount of a modified T cell comprising a CAR and an iCAR. The CAR comprises an antigen-binding domain capable of binding CD19, a transmembrane domain, and an intracellular domain, and the iCAR comprises an antigen-binding domain capable of binding a mural cell (e.g. pericyte or vSMC), and an inhibitory signaling domain.

Neurotoxicity refers to damage to the brain or peripheral nervous system caused by exposure to natural or man-made toxic substances. These toxins can alter the activity of the nervous system in ways that can disrupt or kill nerves. Nerves are essential for transmitting and processing information in the brain, as well as other areas of the nervous system. Symptoms of neurotoxicity include, but are not limited to, paralysis or weakness in the limbs, altered sensation, tingling and numbness in the limbs, headache, vision loss, loss of memory and cognitive function, uncontrollable obsessive and/or compulsive behavior, behavioral problems, sexual dysfunction, depression, loss of circulation, imbalance, and flu-like symptoms. One way of measuring neurotoxicity is by assessing the clinical manifestations of these symptoms listed above. Additional methods of assessing neurotoxicity include, but are not limited to, using laboratory models, e.g., in mice as set forth in the examples.

### E. Nucleic Acids and Expression Vectors

The present disclosure provides a nucleic acid encoding a CAR and/or an iCAR. In one embodiment, a nucleic acid of the present disclosure comprises a nucleic acid sequence encoding an iCAR (e.g., a mural cell-specific iCAR). In one embodiment, a nucleic acid of the present disclosure comprises a nucleic acid sequence encoding a CAR (e.g., a CD19 CAR). In one embodiment, a nucleic acid of the present disclosure comprises a nucleic acid sequence encoding an iCAR (e.g., mural cell-specific iCAR) and a CAR (e.g., a CD19 CAR).

In some embodiments, a nucleic acid of the present disclosure is provided for the production of a CAR and/or iCAR as described herein, e.g., in a mammalian cell. In some embodiments, a nucleic acid of the present disclosure provides for amplification of the CAR and/or iCAR-encoding nucleic acid.

In some embodiments, a nucleic acid of the present disclosure comprises a nucleic acid comprising a CAR coding sequence and an iCAR coding sequence separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. For example, a linker for use in a nucleic acid of the present disclosure comprising a CAR coding sequence and an iCAR coding sequence, allows for the CAR and iCAR to be translated as a polyprotein that is cleaved into separate CAR and iCAR components.

In some embodiments, the linker comprises a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention.

In some embodiments, the linker comprises a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use in the present invention.

In some embodiments, the linker further comprises a nucleic acid sequence that encodes a furin cleavage site. Furin is a ubiquitously expressed protease that resides in the trans-Golgi and processes protein precursors before their secretion. Furin cleaves at the COOH-terminus of its consensus recognition sequence. Various furin consensus recognition sequences (or “furin cleavage sites”) are known to those of skill in the art, including, without limitation, Arg-X1-Lys-Arg (SEQ ID NO:25) or Arg-X1-Arg-Arg (SEQ ID NO:26), X2-Arg-X1-X3-Arg (SEQ ID NO:27) and Arg-X1-X1-Arg (SEQ ID NO:28), such as an Arg-Gln-Lys-Arg (SEQ ID NO:29), where X1 is any naturally occurring amino acid, X2 is Lys or Arg, and X3 is Lys or Arg. A skilled artisan will be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding Furin and F2A, a linker comprising a nucleic acid sequence encoding Furin and E2A, a linker comprising a nucleic acid sequence encoding Furin and P2A, a linker comprising a nucleic acid sequence encoding Furin and T2A. Those of skill in the art would be able to select the appropriate combination for use in the present invention. In such embodiments, the linker may further comprise a spacer sequence between the Furin and 2A peptide. Various spacer sequences are known in the art, including, without limitation, glycine serine (GS) spacers such as (GS)ₙ, (GSGGS)ₙ (SEQ ID NO:1) and (GGGS)ₙ (SEQ ID NO:2), where n represents an integer of at least 1. Exemplary spacer sequences can comprise amino acid sequences including, without limitation, GGSG (SEQ ID NO:4), GGSGG (SEQ ID NO:5), GSGSG (SEQ ID NO:6), GSGGG (SEQ ID NO:7), GGGSG (SEQ ID NO:8), GSSSG (SEQ ID NO:9), and the like. Those skilled in the art would be able to select the appropriate spacer sequence for use in the present invention.

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid encoding a CAR and/or iCAR is operably linked to a promoter. In certain embodiments, the promoter is a phosphoglycerate kinase-1 (PGK) promoter.

For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched on through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR and/or iCAR inducible expression cassette. In one embodiment, the CAR and/or iCAR inducible expression cassette is for the production of a transgenic polypeptide product that is released upon CAR and/or iCAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

In some embodiments, the nucleic acids provided herein may be present in an expression vector and/or a cloning vector. An expression vector typically comprises a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentiviral vector, a gamma retroviral vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In some embodiments, an expression vector (e.g., a lentiviral vector) may be used to introduce the CAR and/or iCAR into an immune cell or precursor thereof (e.g., a T cell). Accordingly, an expression vector (e.g., a lentiviral vector) of the present invention may comprise a nucleic acid encoding for a CAR and/or iCAR. In some embodiments, the expression vector (e.g., lentiviral vector) will comprise additional elements that will aid in the functional expression of the CAR and/or iCAR encoded therein. In some embodiments, an expression vector comprising a nucleic acid encoding for a CAR and/or iCAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR and/or iCAR encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR and/or iCAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR and/or iCAR of the present disclosure. Methods for introducing RNA into a host cell are known in the art. See, e.g., Zhao et al. Cancer Res. (2010) 15: 9053. Introducing RNA comprising a nucleotide sequence encoding a CAR and/or iCAR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR and/or iCAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

### F. Sources of Cells

Prior to expansion, a source of cells (e.g. immune cells; e.g. T cells) is obtained from a subject for ex vivo manipulation. Sources of target cells for ex vivo manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods of treatment disclosed herein include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD 14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD1 1b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR/CD3 complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the T cells are provided as one component within a population of peripheral blood mononuclear cells (PBMCs), cord blood cells, a population comprising T cells, or a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### G. Expansion of Immune Cells

Whether prior to or after modification of cells to express a CAR and/or iCAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the T cells expand in the range of about 20-fold to about 50-fold.

Following culturing, the T cells can be incubated in cell medium in a culture apparatus for a period of time or until the cells reach high cell density for optimal passage before passing the cells to another culture apparatus. The culturing apparatus can be of any culture apparatus commonly used for culturing cells in vitro. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-β, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the T cells expand in the range of about 20-fold to about 50-fold, or more. In one embodiment, human T cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### H. Pharmaceutical Compositions and Formulations

Also provided are populations of immune cells of the invention, compositions containing such cells and/or enriched for such cells, such as in which cells expressing the CAR and/or iCAR make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for intravenous, intraperitoneal, or subcutaneous administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in Experiments 1-4 are now described.

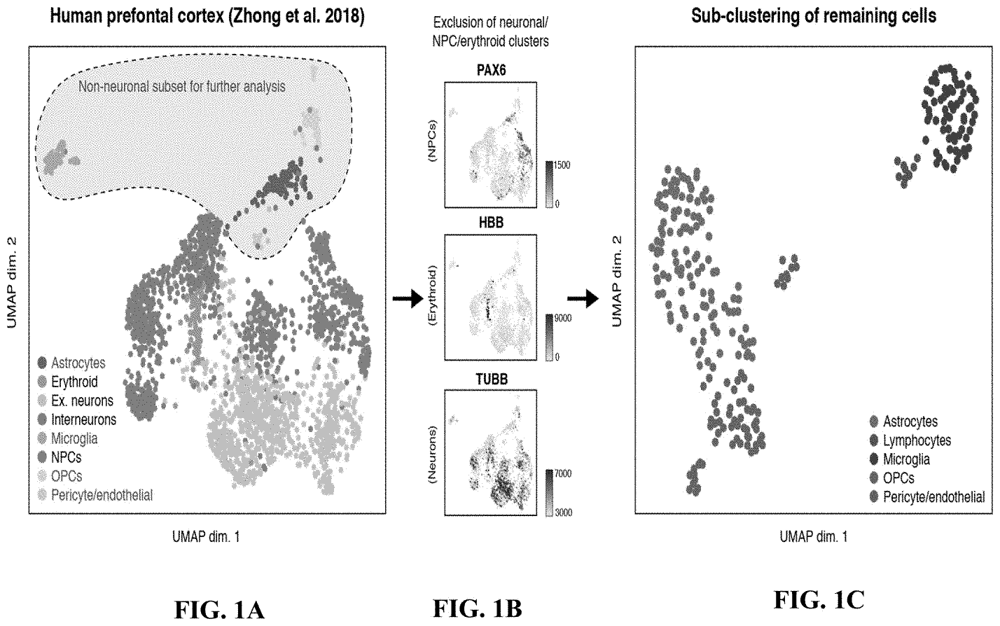

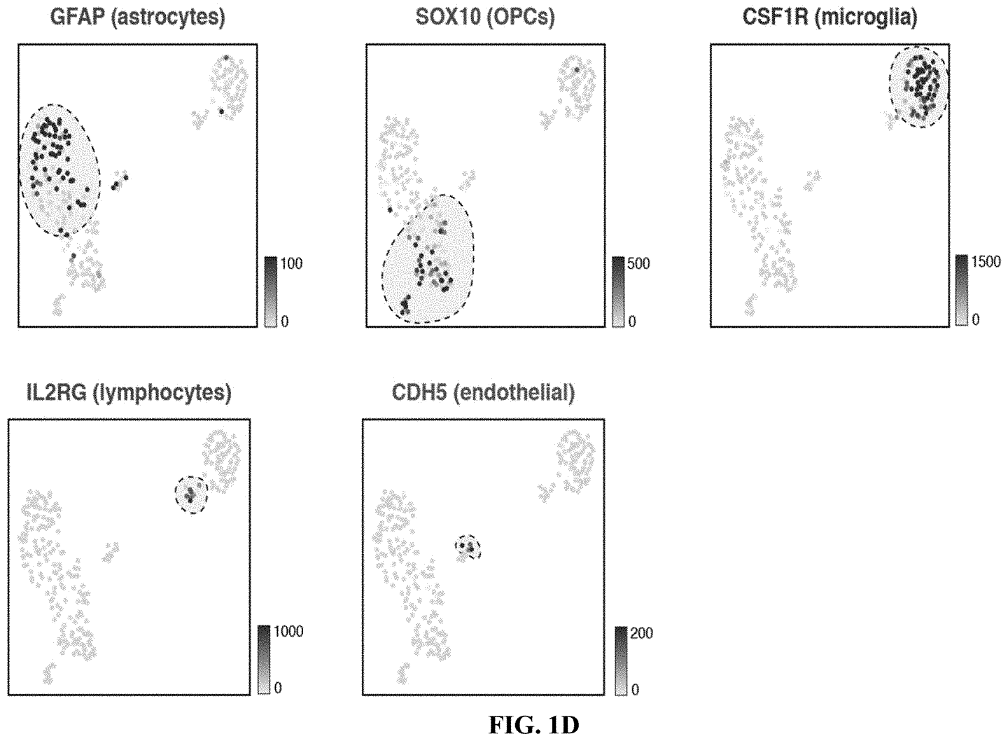

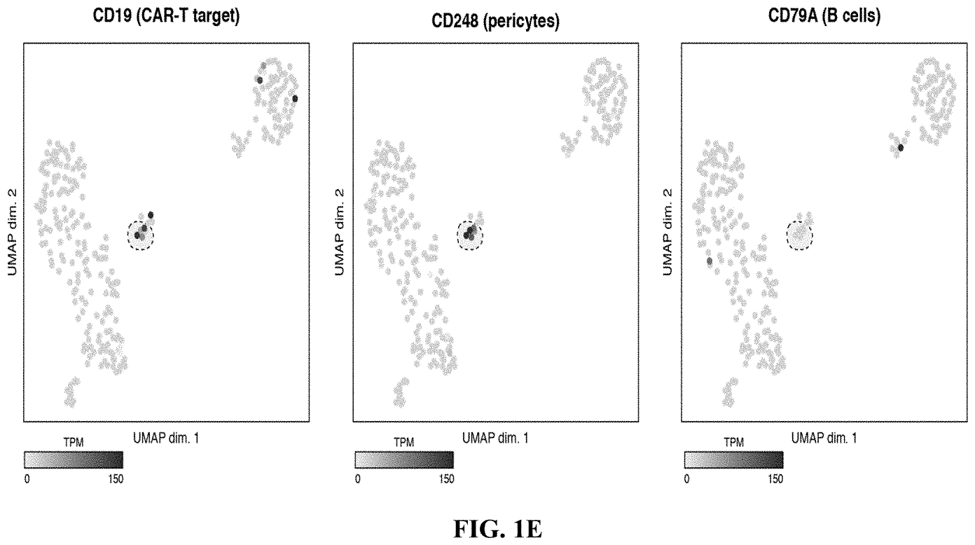

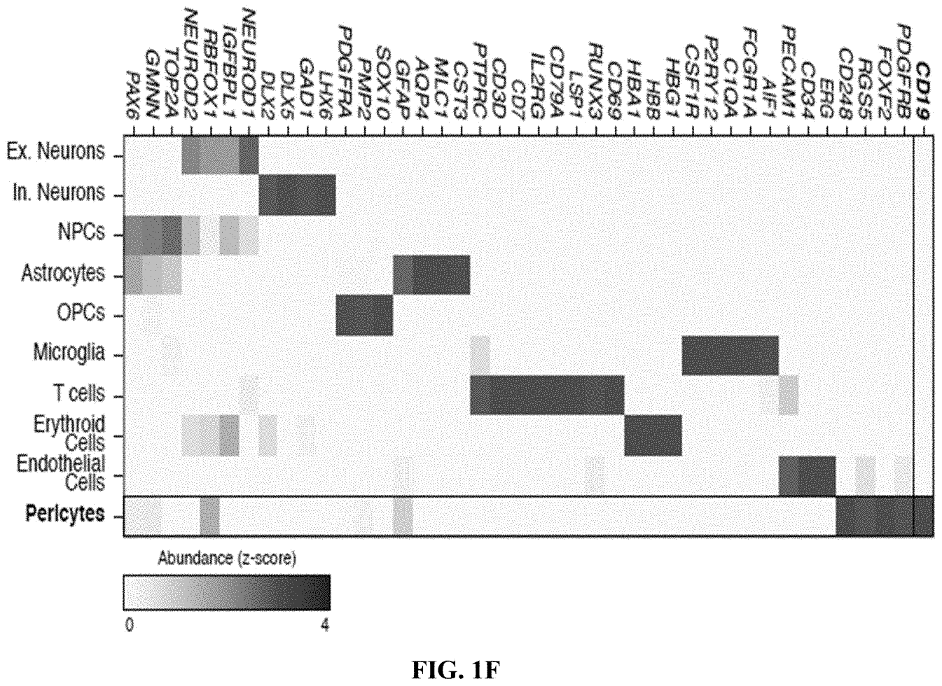

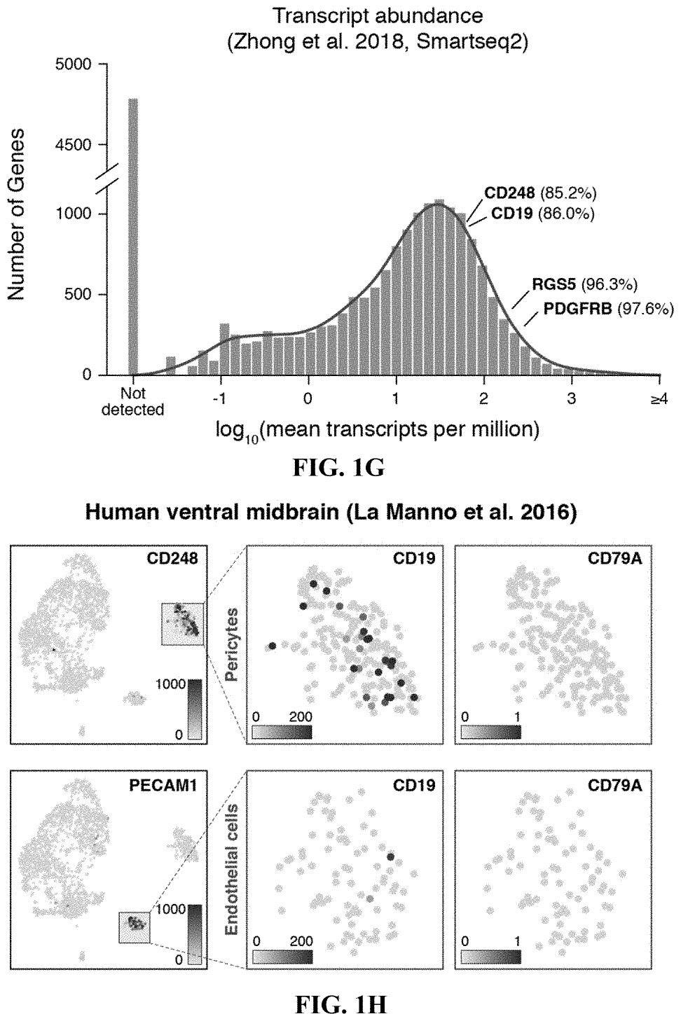

Analysis of human brain single-cell RNA-sequencing data: Processed sequencing data (gene counts per cell) were downloaded as follows: Zhong, et al. 2018, Nature, 555, 524-528. (GEO GSE104276), La Manno et al., 2016, Cell, 167, 566-580. (GEO GSE76381), La Manno et al., 2018, Nature, 560, 494-498. (PanglaoDB database, Karolinska Institutet). Samples were processed using Seurat (Butler, et al., 2018, Nature Biotechnology, 36, 411-420) version 2.3.4 and Scanpy (Wolf, et al., 2018, Genome Biology, 19, 15) version 1.3.1. Cells with fewer than 500 detected genes or UMI counts were excluded, and cell counts were normalized per cell. The ˜1500-2500 most variable genes were used for clustering based on the variance to mean ratio. As the datasets include both post-mitotic and actively cycling cells, the cell cycle status was computed using the CellCycleScoring function and subsequently regressed out using the ScaleData function in Seurat. Principle component analysis was performed using the genes identified as highly variable for each dataset, and the top ˜25-50 principle components were used for subsequent dimensionality reduction using the UMAP algorithm. Clusters were called using the FindClusters function 146 in Seurat, and marker genes for each cluster were identified using the FindMarkers function. Clusters were subsequently manually annotated by comparing highly enriched genes to known cell-type markers. For the analysis of Zhong et al. 2018 data, neuronal precursor cells, erythroid cells, and neuronal cells were identified and excluded, and the remaining cells were subsequently re-clustered. Gene expression data shown in FIG. 1C represents mean expression across all cells with a given cluster label. Percentages and cell counts of a given label (e.g., pericytes) represent the total number of cells in a given cluster, not necessarily only those cells positive for an individual marker, unless otherwise indicated.

Cell lines and cultures: Murine A-20 B cell leukemia cell line was provided by Jong Lee. Nalm6 and A20 tumor cell lines were cultured in RPMI-1640 media supplemented with 10% FBS, penicillin (100 U/mL; Gibco), streptomycin sulfate (100 mg/mL; Gibco), and 10 mmol/L HEPES (Gibco) (R10). 50 μM of 2-mercaptoethanol was added in the culture media of A20 cell line.

Vector constructs: The anti-hCD19-BBz CAR was constructed previously (Milone, et al., 2009, Molecular Therapy, 17, 1453-1464). The mCD19-BBz and mCD19-28z CARs were constructed by ligating the mCD19 scFv (1D3) into the CAR backbone sequences of pTRPE-BBz and pTRPE-28z. Third generation lentiviral vectors were produced as previously described.

Flow cytometry: For the mouse CNS stromal fraction characterization experiments, the following antibodies against mouse molecules were used: anti-mouse CD19 (clone 6D5 0.1 mg/ml, BV650, Biolegend, dilution 1 to 200), CD45 (clone 30-F11, APC-Fire750 Biolegend, dilution 1 to 100), CD31 (clone 390, PerCP-Cy5.5, Biolegend, dilution 1 to 400), B220 (clone RA3-6B2, PE/Dazzle 594 Biolegend, dilution 1 to 200), Zombie Aqua Fixable Viaiblity kit (dilution 1 to 400). Human T cell viability was determined by staining with LIVE/DEAD Fixable Violet Dead Cell Stain Kit (Invitrogen), followed by surface antibody staining. CAR expression was detected using biotinylated protein L (Genscript) followed by R-phycoerythrin-conjugated streptavidin (BD cat. #554061). All experiments were acquired on a BD LSR Fortessa flow cytometer (Becton Dickinson) and data was analyzed with FlowJo v10.

In vitro T cell transduction and cultures: Human CAR-T cells were produced from normal donor T cells provided by the University of Pennsylvania Human Immunology Core, as previously described (Milone, et al., 2009, Molecular Therapy, 17, 1453-1464). Cells were transduced with lentiviral vector encoding anti-human (h) or murine (m) CD19 scFv fused to CAR backbones containing either human 4-1BB or human CD28 and CD3zeta (CD247) signaling domains, as described and were expanded ex vivo for 11 days. Two cell expansions were produced from 2 different healthy human donors. The transduction efficiencies ranged from 20-40%.

Cytotoxicity assays: All cytotoxicity assays were flow cytometry-based and adapted from the Quah et al., 2007, Nature Protocols, 2, 2049-2056. Protocol. Target cells were stained with CFSE (Thermo Fisher) and T cell effectors were stained with Cell Trace Far Red (Thermo Fisher) prior to co-culture. 24 hours after co-culture, cells were harvested, stained for viability with LIVE/DEAD Fixable Violet Dead Cell Stain Kit and analyzed by flow cytometry.

Enzyme-Linked Immunosorbent Assays: For cytokine production assays, effector and target co-culture supernatants were collected after 24 hours and assessed for human IFN-7 using the Human IFN-γ DuoSet ELISA Development Kit (R&D Systems) per manufacturer instructions.

Mouse models: All animal studies adhered to the NIH Guide for the Care and Use of Laboratory Animals and in accordance with protocols approved by The University of Pennsylvania Institutional Animal Care and Use Committee. NOD/scid/IL2rg (NSG) mice were obtained from the University of Pennsylvania Stem Cell and Xenograft Core and housed in the vivarium at the University of Pennsylvania under BSL2 and pathogen-free conditions. 1×10⁷ T cells with 20% normalized CAR+ expression were administered to non-tumor bearing mice by one intravenous tail vein injection in 100 μL of PBS. C57BL/6J mice were obtained from Jackson Laboratory and housed in a BSL1 mouse facility at the University of Pennsylvania. Murine splenocytes from donor mice were harvested, activated with murine-specific anti-CD3/anti-CD28 magnetic beads (Thermo Fisher), and transduced with retroviral vector encoding mCD19mBBz CAR, mCD19mCD28z CAR, or hCD19mBBz CAR transgenes. Four days after transduction, 5×10⁶ murine CAR-T cells were injected intravenously in recipient mice+/−50 mg/kg cyclophosphamide for lymphodepletion.

Blood brain barrier permeability assays: Evans Blue dye (EBD) infusion was performed as previously described (Radu, et al., 2013, Journal of Visualized Experiments, e50062). Seven days after CAR-T cell infusion, mice were injected intravenously with EBD+/−mannitol. Thirty minutes after EBD infusion, mice were euthanized; brain tissues were excised and fixed in 10% formalin solution for at least 24 h. After washing and dehydrating, brains were embedded in paraffin and cross-sectioned at a thickness of 5 μm. Sections were deparaffinized, mounted with DAPI-containing Vectashield, and analyzed by fluorescence microscopy.

Immunohistochemistry: The human anti-CD19 antibody (clone BT51E) was used to stain normal human brain. Images were visualized and acquired using the Leica #NCL-L-CD19-163 microscope. Automated immunohistochemistry was performed with Ventana Benchmark XT following a clinically-validated protocol for CD19. 4-micron FFPE tissue sections were deparaffinized and rehydrated. Antigen retrieval was performed using Standard Cell Condition 1 (pH 8.5) for 60 minutes (Ventana Medical Systems). Slides were incubated with anti-human CD19 (1:50, monoclonal, Abnova BT51E) at 37° for 30 minutes. The ultraView Universal DAB Detection system (Ventana) was used with 3,3′-diaminobenzidine chromogen.

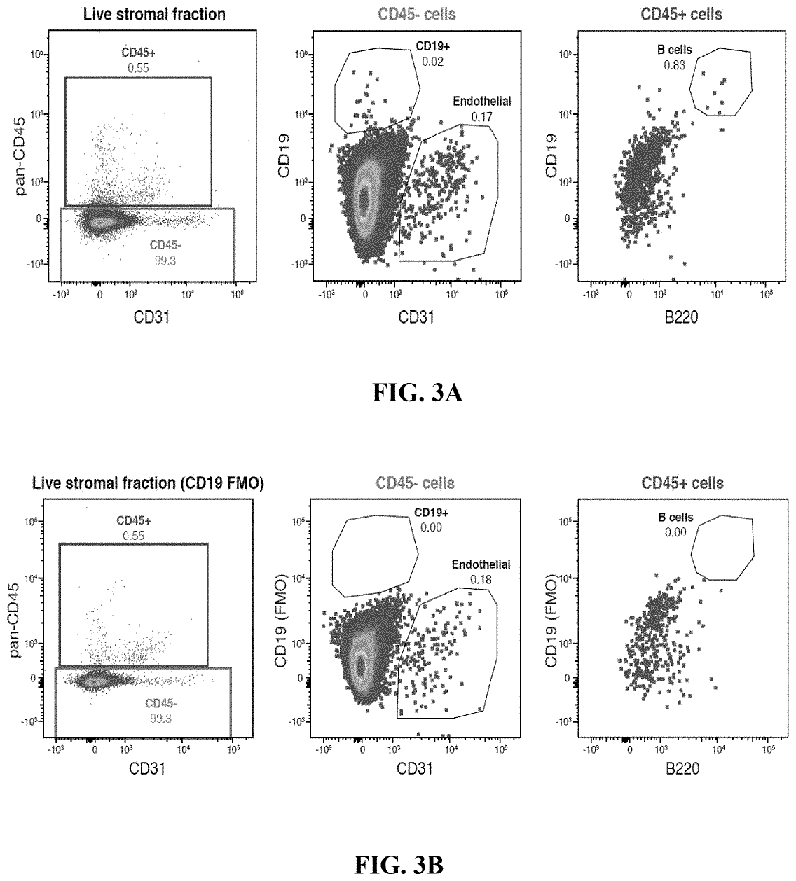

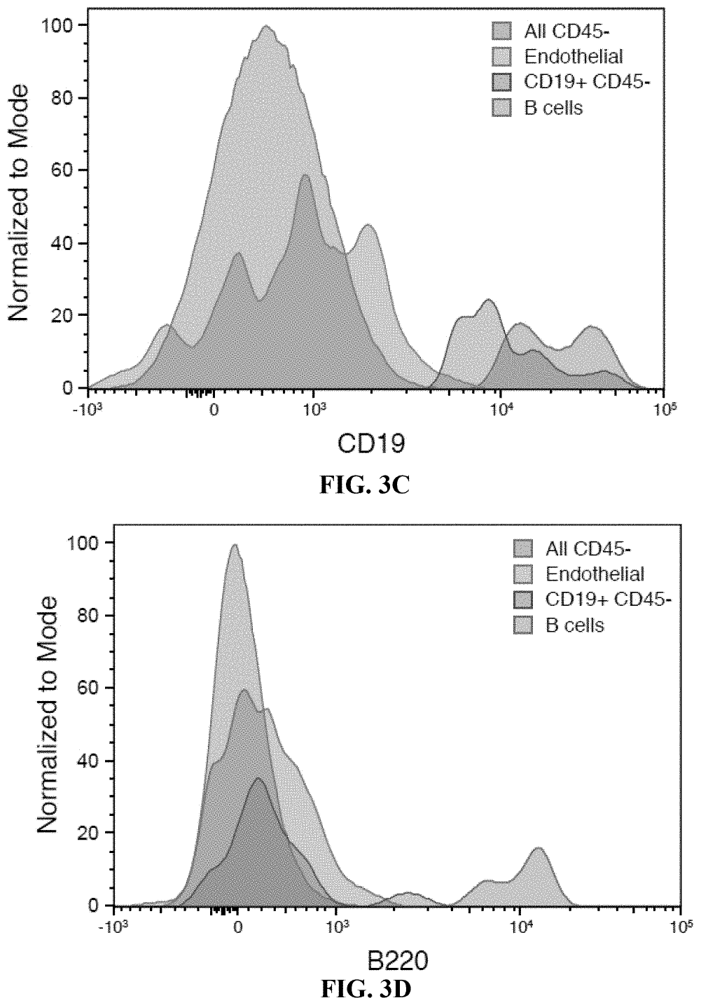

High resolution in vivo brain MRI: Mice were anesthetized by using isoflurane maintained at 0.5-1.5% in 1 liter/min air. The lateral tail vein was cannulated to administer gadodiamide (287 mg/ml) or mannitol (25% w/v). Mice were transferred to a 9.4T horizontal bore small animal MR scanner (Bruker, Billerica, Mass.) and placed in a 20 mm diameter commercial quadrature proton coil (m2m Imaging Corp., Cleveland, Ohio). During the MR scans, animal body temperature was maintained with the air generated and blowing through a heater (SA Instruments, Inc., Stony Brook, N.Y.). Respiration and body temperature were continuously monitored using an MRI-compatible small animal monitoring system (SA Instruments, Inc., Stony Brook, N.Y.). The T1-mapping was performed pre- and post-gadodiamide injection on 0.7 mm thick coronal slice in the mid-brain using saturation recovery protocol with following parameters, number of averages=2, field of view=20′20 mm2, matrix size=128′128, echo time=8 ms, repetition times=200, 500, 800, 1200, 1500, 4000 and 9000 ms, scan time 30 minutes. Following the T1 acquisition, gadodiamide was administered via tail vein as described previously (Ku, et al. 2018, Methods in Molecular Biology, 1718, 395-408.) T1 map was generated using the image sequence analysis tool in Paravision 6.0.1 by exponential fitting of the signal recovery data. An in-house MATLAB script was used to quantity the DTi (T1Post−T1Pre) caused by Gd retention in the brain. Representative −DTi maps from the brains of a mouse in each group are plotted in the figure. 3×10⁷ T cells with 32% normalized CAR+ expression were administered to non-tumor bearing NSG mice (n=2 per group) by one intravenous tail vein injection in 100 μL of PBS. All the MRI imaging experiments were performed 4 days post CAR-T cell infusions.

The results of the experiments are now described.

### Example 1

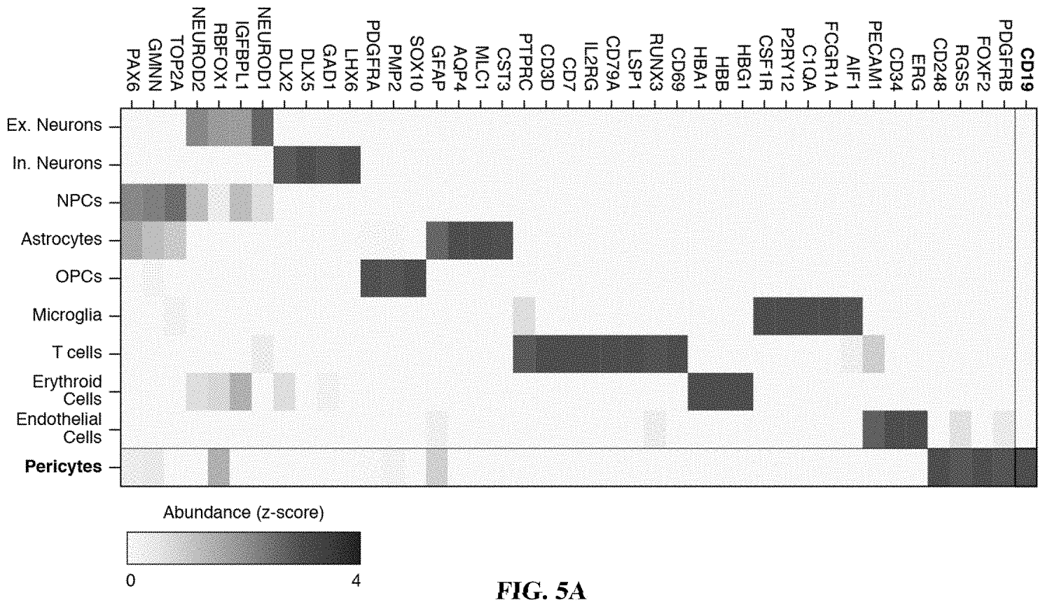

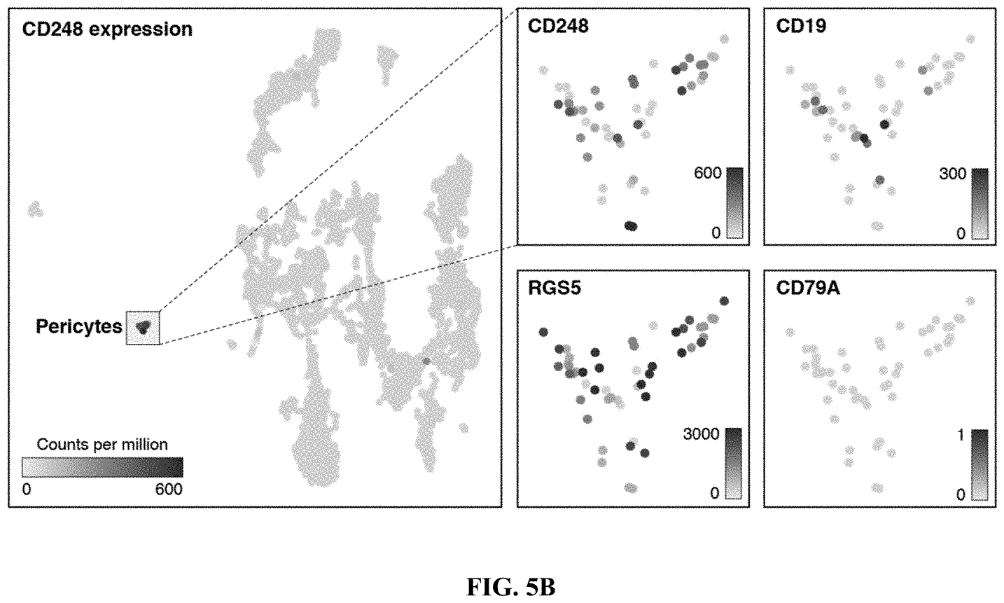

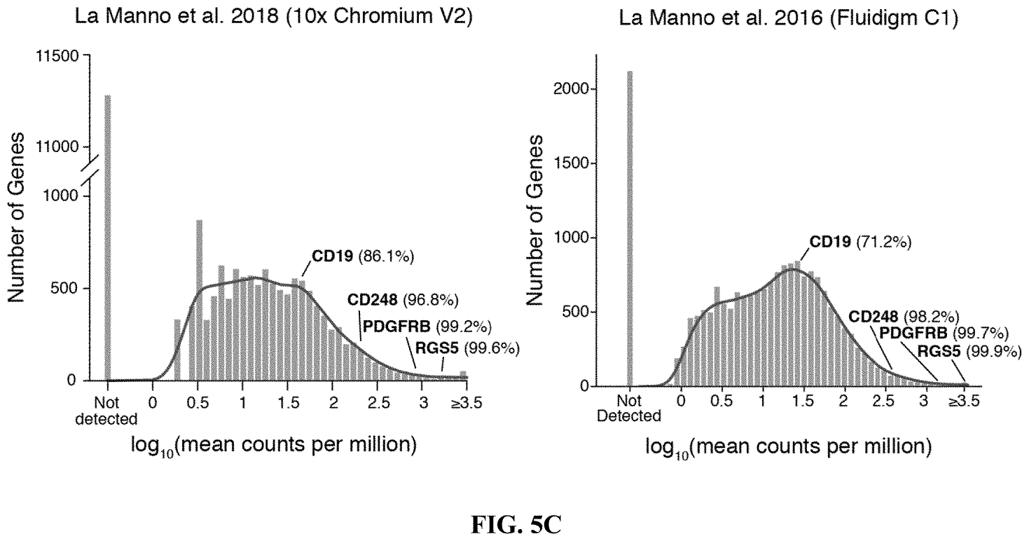

One possible mechanism for neurotoxicity is the unanticipated expression of CD19 on non-B cells in the brain. It was hypothesized that if such a population of CD19-expressing cells were present, it might be identifiable in data generated from recent efforts to map the human brain transcriptome with single-cell resolution. Thus, scRNA-seq data from 2,364 human prefrontal cortex cells were analyzed (Zhong, et al. 2018, Nature, 555, 524-528) (FIG. 1A). Cells were clustered and broad populations were identified, focusing subsequent analyses on non-neuronal, non-erythroid cells. These groups further segregated into astrocyte, lymphocyte, microglial, oligodendrocyte precursor, endothelial, and pericyte populations (FIG. 1B-1C). These populations were identified on the basis of the expression of canonical marker genes: pericytes specifically expressed expected marker genes, such as PDGFRB, FOXF2, RGS5, and CD248, while endothelial cells expressed a distinct set of markers, such as CD34 and PECAM1 (CD31) (FIG. 5A). This analysis revealed a small population of cells (˜1.5% of non-neuronal cells; ˜0.2% including neuronal cells) that expressed CD19 and co-expressed the pericyte marker CD248 (FIG. 1D). Pericytes wrap endothelial cells and regulate angiogenesis, wound healing, and contribute to maintaining the blood brain barrier. Importantly, this population was negative for the B cell marker CD79A, arguing against the possibility of artifactual CD19 expression due to B cell-pericyte doublets (FIG. 1D). A separate population of lymphocytes was observed that expressed the marker genes CD45 (PTPRC), CD3, CD7, and IL2RG (CD132), representing a population of T cells (FIG. 1C-1D, FIG. 5A). Notably, the expression of lymphocyte markers and pericyte markers were mutually exclusive in the observed clusters, and the expression of CD19 was specific to the pericyte cluster (FIG. 5A).

This observation was then reproduced in additional independent scRNA-seq datasets from the human brain. In a dataset from human forebrain (La Manno et al., 2018, Nature, 560, 494-498), a pericyte population (48/7906 cells) positive for the markers CD248 and RGS5 also displayed expression of CD19 (12/48 cells had measurable CD19) and the absence of the B-cell marker CD79A (FIG. 1D). In a third dataset derived from human ventral forebrain (La Manno et al., 2016, Cell, 167, 566-580), expression of CD19 was again observed in a population of CD248-positive pericytes (195 pericytes/1977 cells, of which 25/195 had measurable CD19; (FIG. 5B). As with other datasets, no RNA-sequencing counts for CD79A were identified in any pericytes. Although spurious non-pericyte CD19-positive cells were observable, these are likely the result of cell-cell doublets, such as pericyte-endothelial cell doublets observed due to the adhesion of pericyte to endothelial cells in the brain.

Due to the inherent sparsity of single-cell data, even highly expressed genes will not be detected in every cell. Therefore, the level of expression of CD19 in pericytes was compared to known pericyte marker genes relative to the overall gene expression distribution in pericytes. Mean-normalized gene expression values provided a rough estimate of the relative expression of a given gene in a cell population of interest, despite the inherent sparsity of single-cell data. As expected, pericyte markers such as CD248, RGS5, and PDGFRB ranked in the top percentiles of gene expression in pericytes from the human prefrontal cortex (85^{th}, 96^{th}, and 98^{th}, respectively) (FIG. 1F). CD19 ranked in the 86^{th} percentile for gene expression, suggesting relatively robust expression. This finding was repeated, with relatively similar respective gene expression percentiles, in the data from the human forebrain datasets (FIG. 5C).

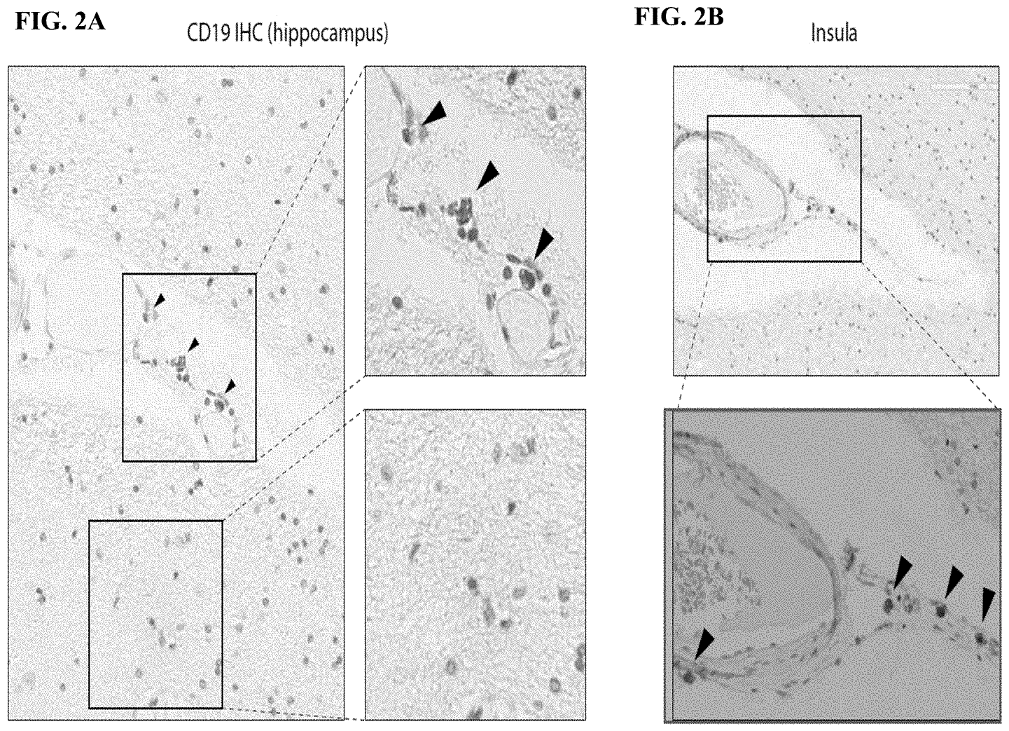

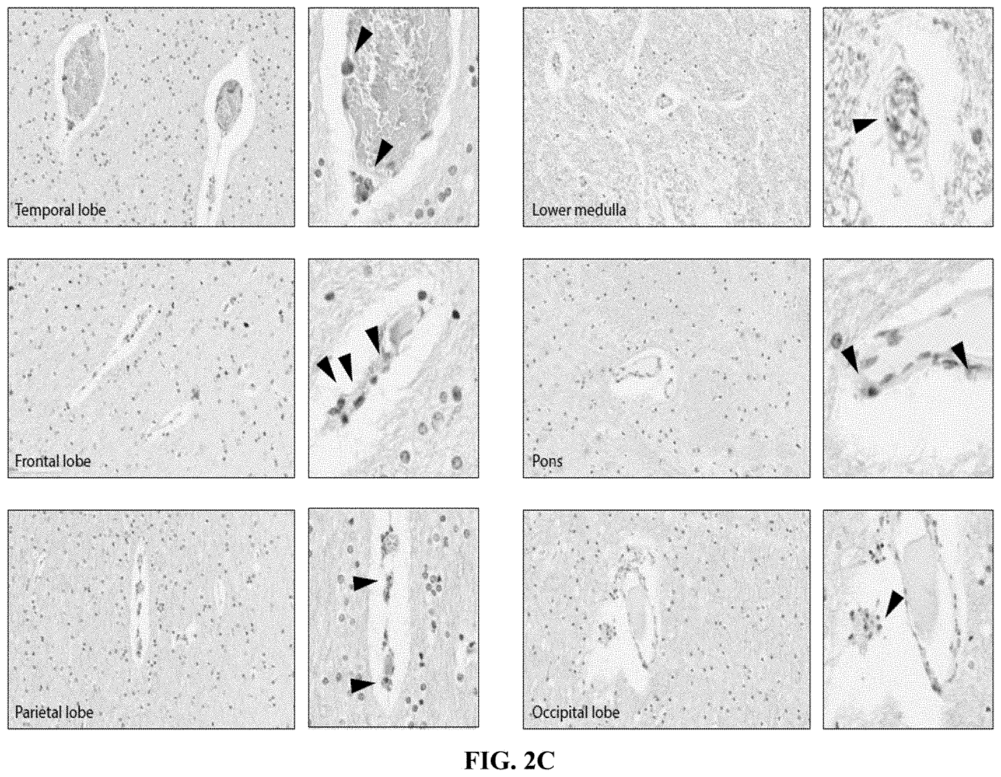

To assess the expression of CD19 protein in human pericytes, immunohistochemistry was performed on several regions of the human brain using a clinically-validated anti-human CD19 antibody (clone BT51E) on samples from healthy deceased subjects. CD19 expression was found on cells present adjacent to the vessel basement membrane walls in perivascular areas (FIG. 2). CD19 expression was observed across multiple brain regions, with particular regions, such as the hippocampus, insula, temporal lobe, frontal lobe, and parietal lobe displaying a comparatively higher, albeit still rare, incidence of CD19-positive cells. In contrast, regions such as the lower medulla, pons, and occipital lobe displayed lower rates of CD19-positive cells (FIG. 2).

To complement these observations, brains from healthy C57Bl/6J were extracted and stromal cells from the brain were isolated as previously described (Boroujerdi, et al., 2014, Methods in Molecular Biology, 1135, 383-392.). Analysis by flow cytometry of live single cells demonstrated the presence of CD19+ cells within the pan-CD45 negative fraction, in addition to CD31+ endothelial cells (FIG. 3A-3B). A population of CD45+ B cells was also identifiable on the basis of CD19 and B220 expression (FIG. 3A-3B). The CD19 expression levels among CD19+, CD45− cells was comparable to that of CD45+, CD19+ B-cells, displaying clear separation from CD19 levels in endothelial cells as well as the overall CD45 negative stromal fraction (FIG. 3C). In contrast, B220 expression was found only in B cells, and not in CD19+, CD45− cells, as expected (FIG. 3D).

### Example 2

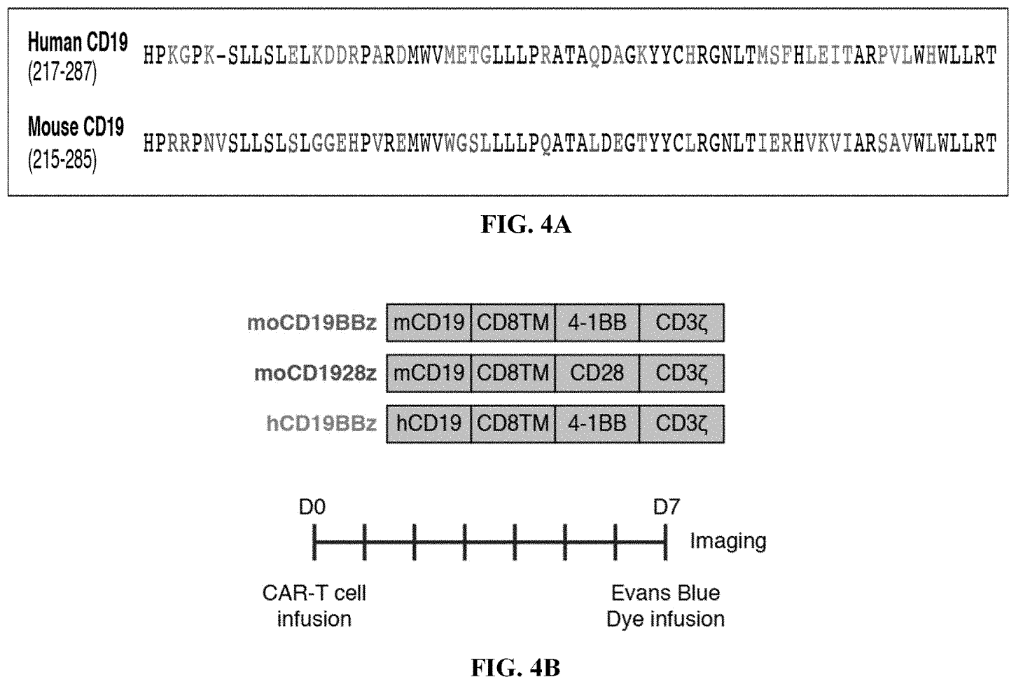

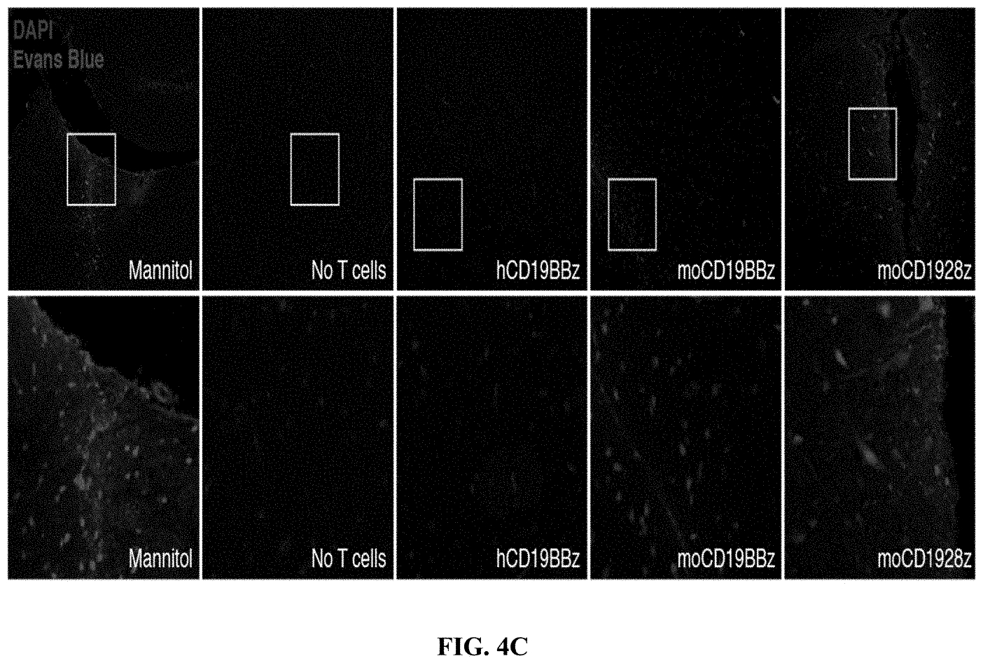

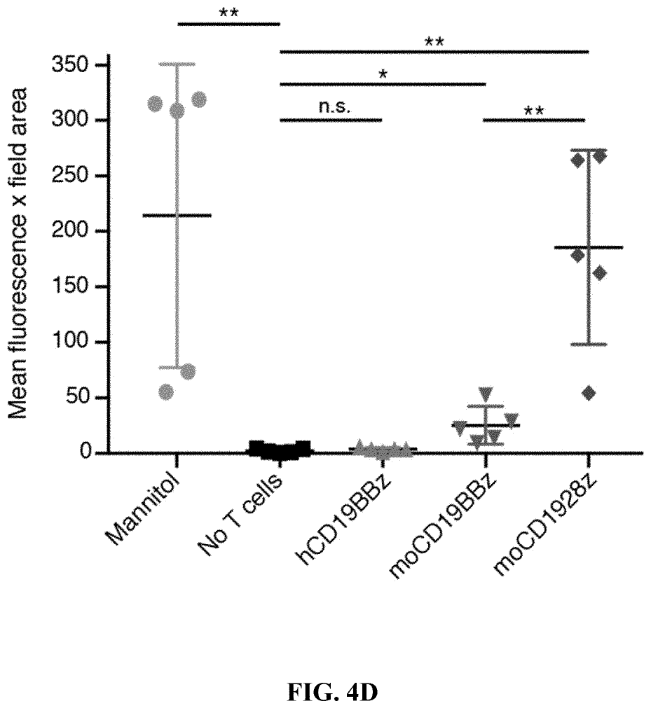

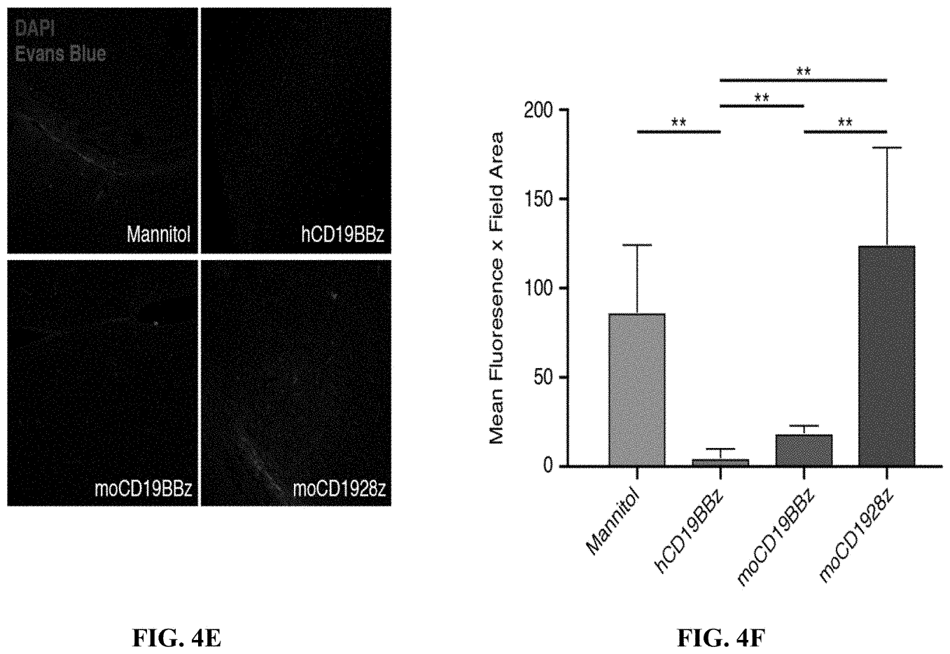

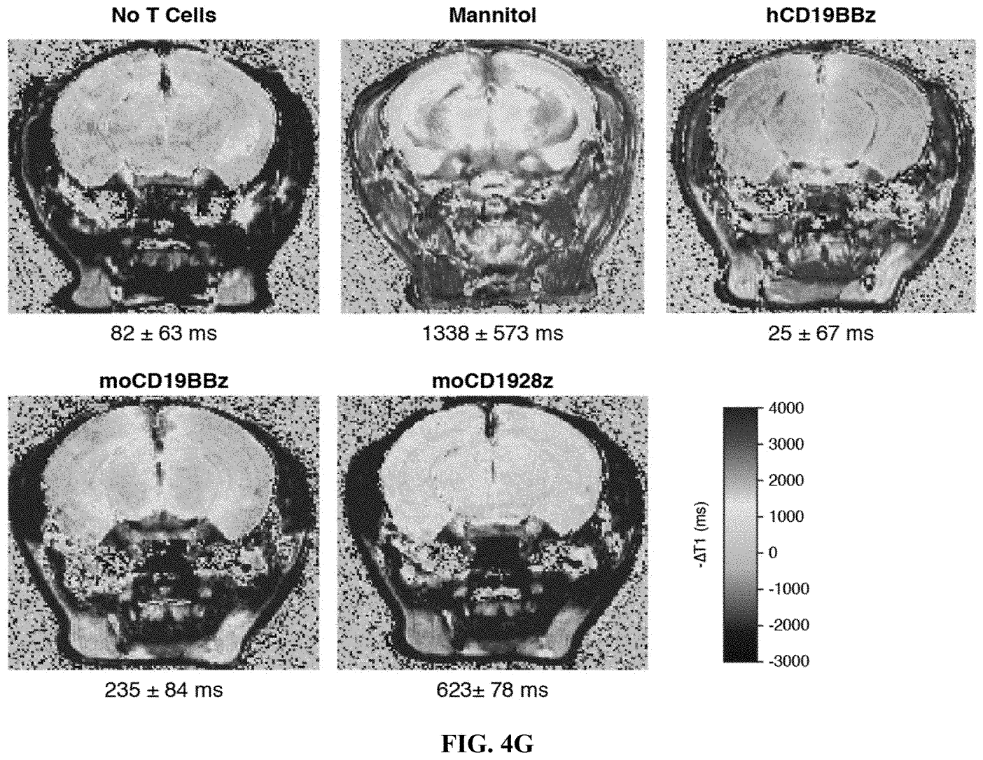

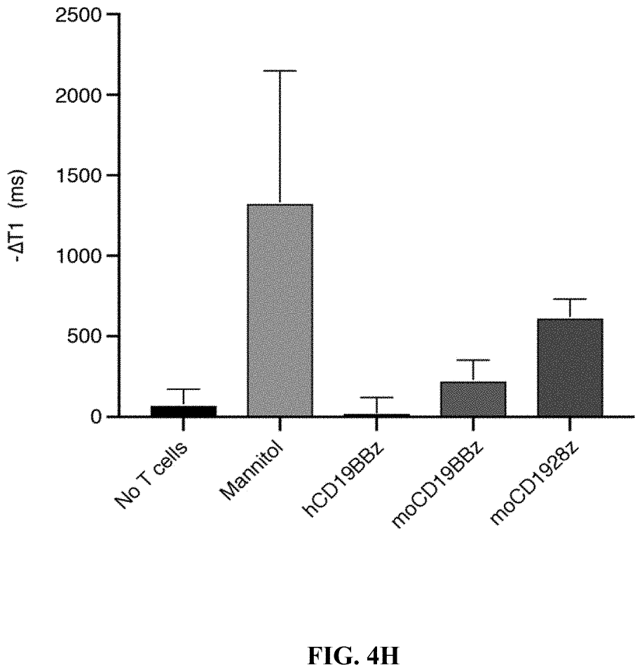

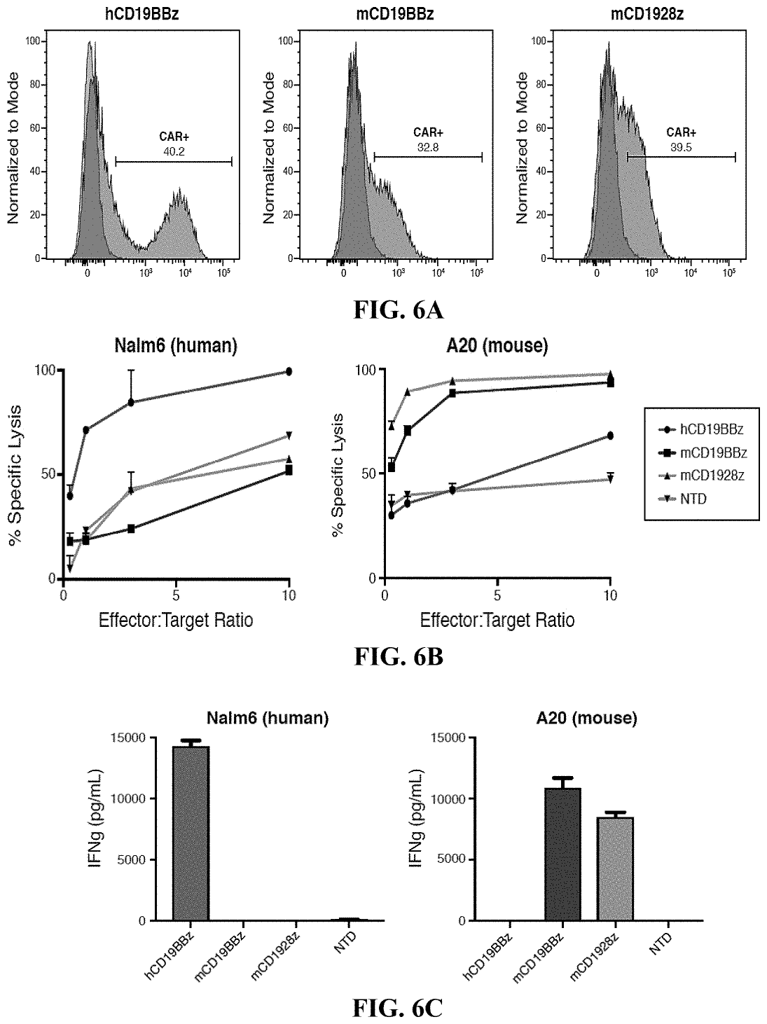

These results demonstrated that CD19-positive cells were present in the brain and appeared both transcriptionally and histologically as pericytes. As CD19-positive non-B cells were also present in the mouse brain, the presence of blood brain barrier disruption in mice lacking a B-cell population that would otherwise control against any blood brain barrier disruption resulting from CRS-related symptoms was assessed. CD28-based or 4-1BB-based CAR-T cells specific for either murine CD19 (1D3 scFv, mCD1928z and mCD19BBz) or human CD19 (FMC63 scFv, hCD19BBz) were generated. The hCD19BBz cells represent a negative control experimental condition, as no CD19-specific targeting would be expected in recipient mice due to the absence of strong sequence homology at the FMC63 epitope targeted by the hCD19BBz cells (FIG. 4A). CAR expression on the cell surface was confirmed (FIG. 6A) and CAR-T cell functionality was tested in vitro using flow cytometric-based cytotoxicity assays using the human CD19+ B-ALL cell line Nalm6 and the murine CD19+ B-ALL cell line A20 as targets. Specific lysis of Nalm6 cells was observed, but not A20 cells, by anti-human CD19 CAR-T cells with increasing effector-to-target ratios (FIG. 6B). Conversely, anti-murine CD19 CAR-T cells lysed A20 at a much higher rate than Nalm6 cells (FIG. 6B). Species specificity of both anti-murine and anti-human CD19 CAR-T cells was confirmed based on IFN-7 secretion (FIG. 6C).

### Example 3

To address whether blood brain barrier disruption is caused by on-target cytotoxicity of brain pericytes, in addition to CRS-related effects caused by B-cell targeting, immunodeficient, non-tumor bearing NSG mice were treated with PBS, human CAR-T cells containing either CD28-based or 4-1BB-based constructs specific for murine CD19, or human 4-1BB-based CAR-T cells targeting human CD19 (FIG. 4B). Seven days post CAR-T cell infusion, mice were infused intravenously with Evans Blue dye (EBD), which allows quantitative measurement of extravasation and can be used to analyze blood brain barrier permeability. EBD binds to albumin, which remains in the bloodstream in normal physiologic conditions. When the blood brain barrier is disrupted, small proteins, such as albumin, can cross. Mice receiving no CAR-T infusion were treated with mannitol and EBD simultaneously as a positive control of blood brain barrier permeability (FIG. 4B). Thirty minutes after EBD injection, mice were euthanized and brains were harvested, formalin-fixed, and paraffin-embedded. Deparaffinized cross sections of brain were stained with DAPI and imaged using confocal microscopy for EBD fluorescence. Mice treated with mannitol displayed EBD fluorescence indicative of blood brain barrier extravasation, in contrast with mice receiving no treatment as well as mice receiving anti-human CD19 CAR-T cells (hCD19BBz) (FIG. 4C). Mice treated with anti-murine mCD19BBz cells, and to a higher extent, anti-murine mCD1928z cells, displayed blood brain barrier extravasation, indicative of disruption of the blood brain barrier independent of any B cell-killing-related effects (FIG. 4C). These results were quantified, displaying a significant enrichment of EBD fluorescence in mCD19BBz and mCD1928z conditions (FIG. 4D).

This animal study was repeated using a syngeneic, immunocompetent C57Bl/6J model with cyclophosphamide as a lympho-depleting preconditioning regimen. In this model, mice were treated with murine T cells expressing murine versions of the CARs evaluated in the NSG model (hCD19BBz, mCD19BBz, or mCD1928z) and analyzed as above. The syngeneic study recapitulated the pattern of blood brain barrier permeability observed in the NSG model, suggesting that the presence of murine CD19+ B cells in the syngeneic model did not affect the specific disruption of the blood brain barrier observed only in the murine-targeting conditions (FIG. 4E-4F).

Blood brain barrier integrity was measured after CAR-T cell treatment using high-definition imaging with 9.4 tesla magnetic resonance imaging (MRI). Immunodeficient, non-tumor bearing NSG mice were treated with PBS, mannitol, hCD19BBz, mCD19BBz, or mCD1928z conditions, as before. Four days post infusion, brain MRI analysis confirmed an increase in gadodiamide uptake in the brain parenchyma in the mCD19BBz and mCD1928z conditions as well as the mannitol control (FIG. 4G-4H). As before, the hCD19BBz condition did not display blood brain barrier disruption, and the CD28-based CAR-T cells displayed increased blood brain barrier disruption relative to the 4-1BB-based CAR-T cells.

These findings are consistent with a role for pericytes in mediating neurotoxicity in patients receiving CD19 CAR-T cells; namely, CD19 CAR-T-mediated depletion of pericytes leads to endothelial activation and increased blood brain barrier permeability. Previous analyses of patients who received CD19 CAR-T therapy and displayed neurologic adverse reactions revealed edema, multifocal hemorrhage, and vascular disruption in the brain, and neuropathologic evaluation of brain from a patient who developed fatal CRS and/or neurotoxicity revealed CAR-T cell infiltration into the brain.

### Example 4

The CD19+ subset was further characterized and the following surface proteins were well-expressed on the CD19+ pericytes, and not on B-cells: BGN—surface & extracellular, biglycan, FN1—surface & extracellular, fibronectin 1, SEMASA—surface, semaphorin 5A, PLXDC1—surface, plexin domain containing 1, THY1—surface, thy 1 surface antigen, CDH6—surface, cadherin, TFPI—surface, tissue factor pathway inhibitor, COL1A2—surface & extracellular, collagen type 1α2 chain, ITGA1—surface, integrin subunit α1, EDNRA—surface, endothelin receptor type A, CSPG4 (aka NG2)—surface, chondroitin sulfate proteoglycan 4, PCDH18—surface, protocadherin 18, CDH11—surface, cadherin 11, AXL—surface, AXL receptor tyrosine kinase, NTM—surface, neurotrimin, TNFRSF1A—surface, S1PR3—surface, sphingosine-1-phosphate receptor, F3—surface and secreted, tissue factor. These pericyte-associated antigens are targets for the iCARs disclosed herein.

The materials and methods employed in Experiments 5-10 are now described.

Cell lines and cultures: The murine A-20 B cell leukemia cell line was kindly provided by Dr. Jong Lee (University of Pennsylvania). Nalm6 and A20 tumor cell lines were cultured in RPMI-1640 media supplemented with 10% FBS, penicillin (100 U/mL; Gibco), streptomycin sulfate (100 mg/mL; Gibco), and 10 mmol/L HEPES (Gibco) (R10). 50 pM of 2-mercaptoethanol was added in the culture media of A20 cell line.

In vitro T cell transduction and cultures: Human CAR-T cells were produced from normal donor T cells provided by the University of Pennsylvania Human Immunology Core, as previously described (Milone et al., 2009, Molecular Therapy, 17, 1453-1464). Cells were transduced with lentiviral vector encoding anti-human (h) or murine (m) CD19 scFv fused to CAR backbones containing either human 4-1BB or human CD28 and CDSzeta (CD247) signaling domains, as described and were expanded ex vivo for 11 days (Milone et al., 2009, Molecular Therapy, 17, 1453-1464). Two cell expansions were produced from 2 different healthy human donors. The transduction efficiencies ranged from 20 to 40%.

Mouse models: All animal studies adhered to the NIH Guide for the Care and Use of Laboratory Animals and in accordance with protocols approved by The University of Pennsylvania Institutional Animal Care and Use Committee. NOD/scid/IL2rg (NSG) mice were obtained from the University of Pennsylvania Stem Cell and Xenograft Core and housed in the vivarium at the University of Pennsylvania under BSL2 and pathogen-free conditions. 1×10⁷ T cells with 20% normalized CAR+ expression were administered to non-tumor bearing mice by one intravenous tail vein injection in 100 L of PBS.

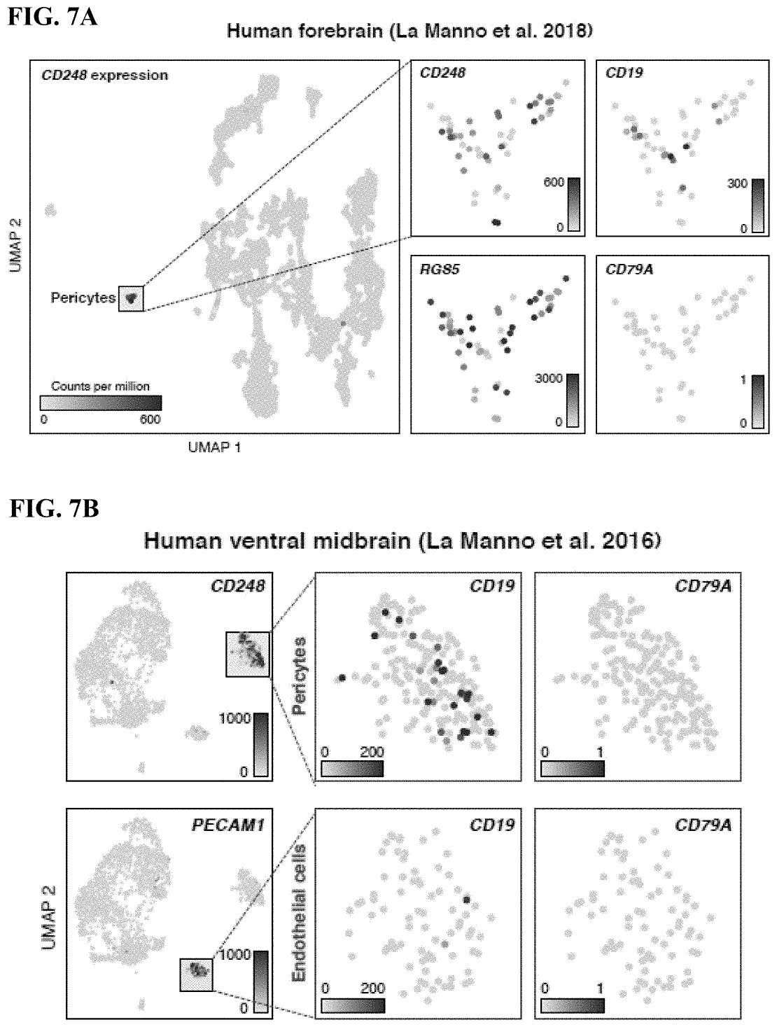

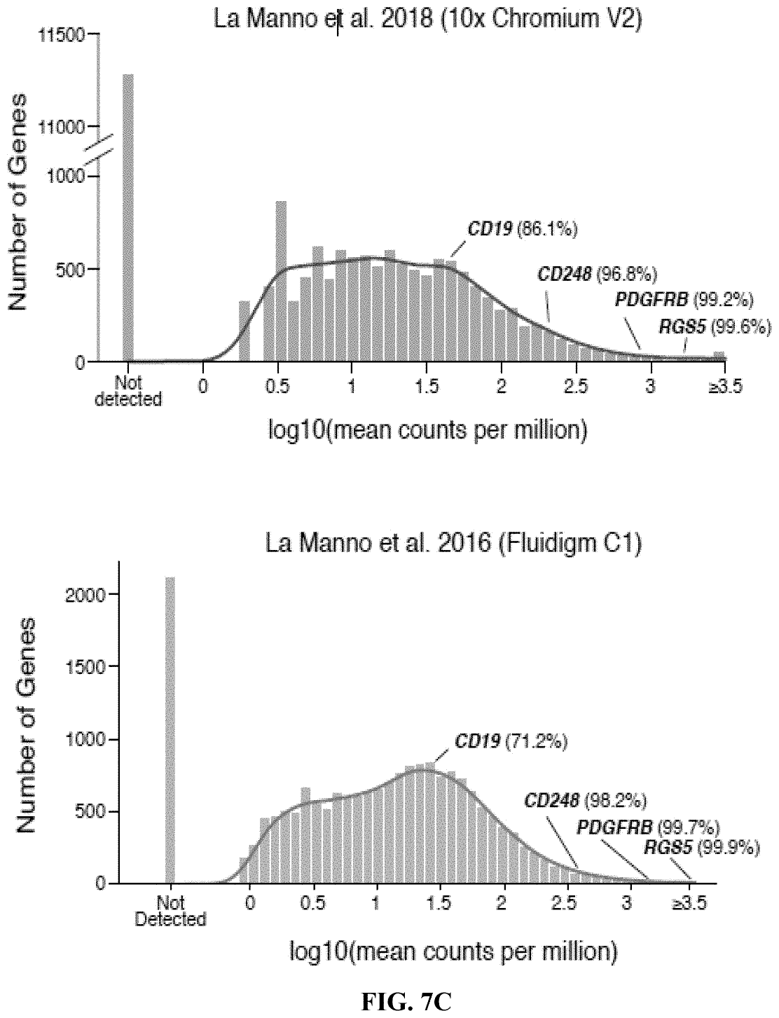

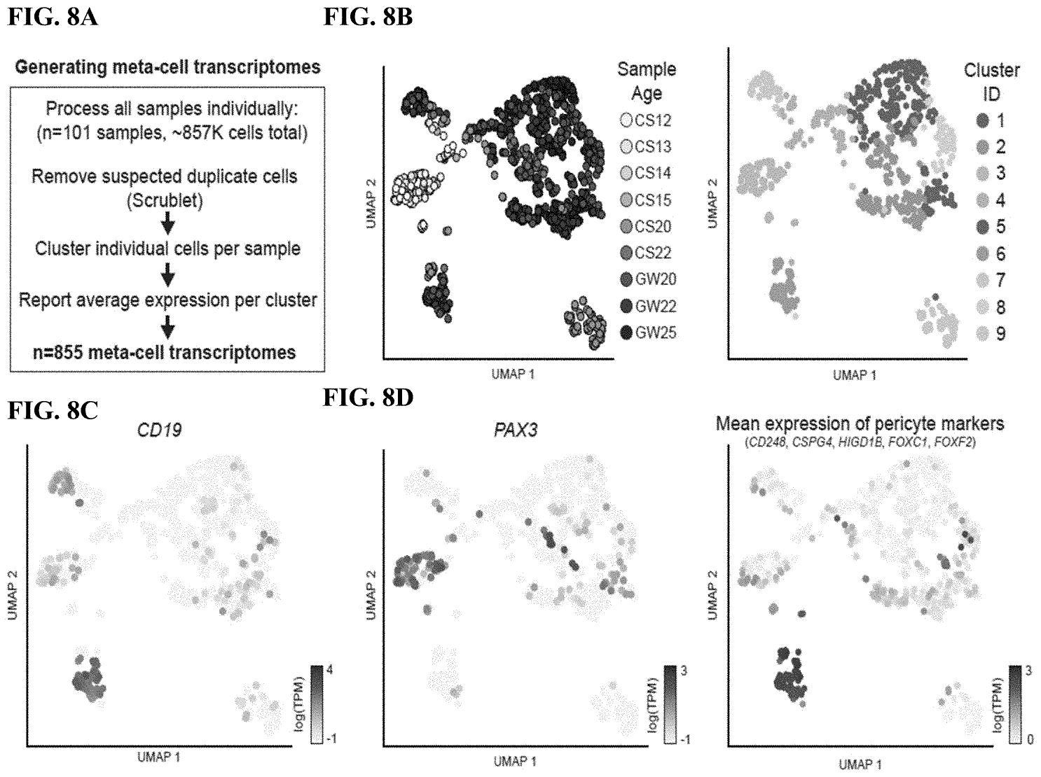

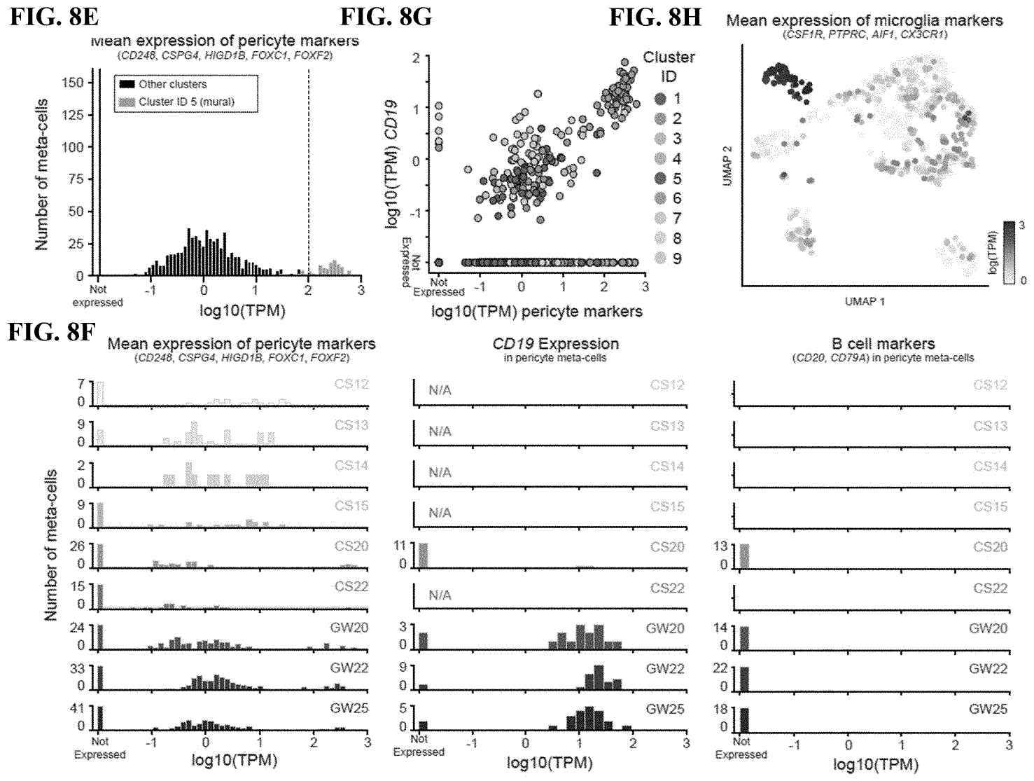

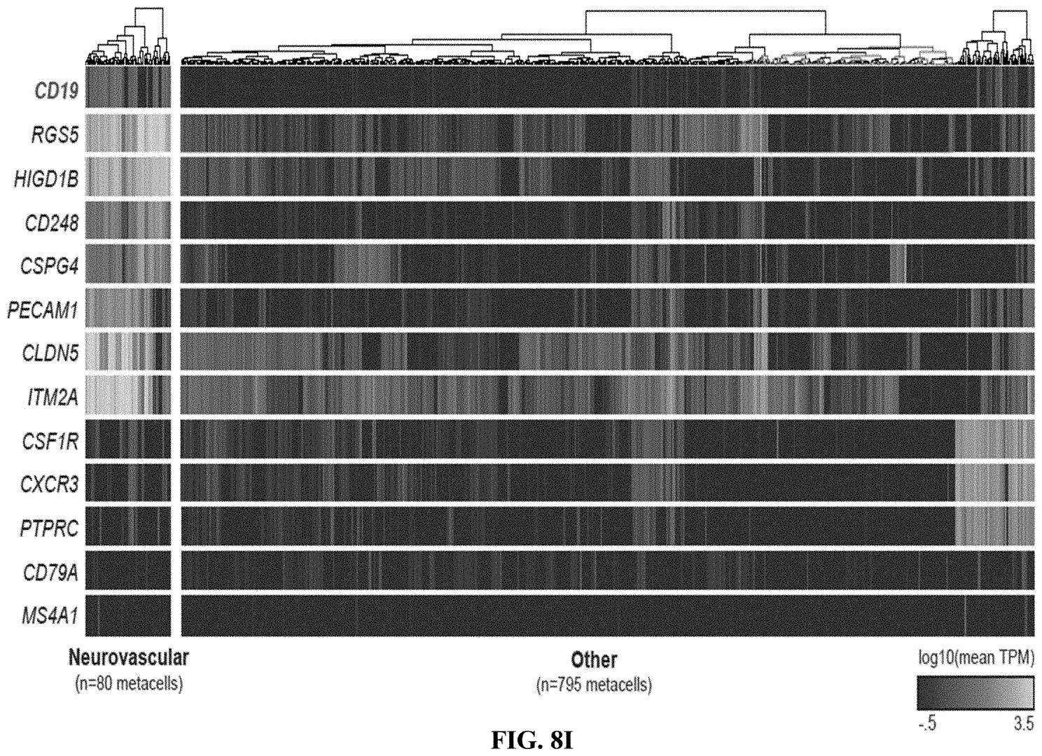

Analysis of human brain single-cell RNA-sequencing data: Sequence data were downloaded as described in Data and Code Availability. For data shown in FIGS. 1A-1H and 7A-7C, samples were processed using Seurat (Butler et al., 2018, Nature Biotechnology, 36, 411-420) version 2.3.4. For data shown in FIGS. 8A-11C, samples were processed using Scanpy version 1.4.3 (Wolf, et al., 2018, Genome Biology, 19, 15) or Seurat version 3.1.4.

Analysis of Zhong 2018, La Manno 2016, La Manno 2018: Cells with fewer than 500 detected genes or UMI counts were excluded, and cell counts were normalized per cell using log(depth-normalized counts+1). The −1500-2500 most variable genes were used for clustering based on the variance to mean ratio. As the datasets include both post-mitotic and actively cycling cells, the cell cycle status was computed using the CellCycleScoring function and subsequently regressed out using the ScaleData function in Seurat. Principle component analysis was performed using the genes identified as highly variable for each dataset, and the top −25-50 principle components were used for subsequent dimensionality reduction using the UMAP algorithm. Clusters were called using the FindClusters function 146 in Seurat, and marker genes for each cluster were identified using the FindMarkers function. Clusters were subsequently manually annotated by comparing highly enriched genes to known cell-type markers. For the analysis of Zhong et al. 2018 data, neuronal precursor cells, erythroid cells, and neuronal cells were identified and excluded, and the remaining cells were subsequently re-clustered. Gene expression data shown in FIGS. 1G and 7C represents mean expression across all cells with a given cluster label. Percentages and cell counts of a given label (e.g., pericytes) represent the total number of cells in a given cluster, not necessarily only those cells positive for an individual marker, unless otherwise indicated.

Comparison of gene expression with B cells: Single-cell gene expression data from B cells was generated from the PBMC data described in Data and Code Availability. A database of extracellular proteins based on mass spectrometry data was used for the analysis of putative cell-surface proteins (Bausch-Fluck et al., 2015, PloS One 10, e0121314). This database includes proteins that may be secreted and thus not strongly enriched at the cell surface, but in the interest of not excluding proteins that might be at the cell surface, this more comprehensive database was used. Gene expression was computed as the mean across all single B cells. To account for differences in the expected distribution of mean gene expression due to the inherent sparsity of single-cell RNA expression data, which is biased based on cell capture and library preparation technique, as well as sequencing depth, the distribution of gene expression values were quantile normalized using the normalize.quantiles function in R to facilitate comparisons across cell types. As such, the absolute gene expression values are pseudo-arbitrary, in that they have been transformed to be relatively comparable across different cell types from distinct single-cell sequencing techniques.

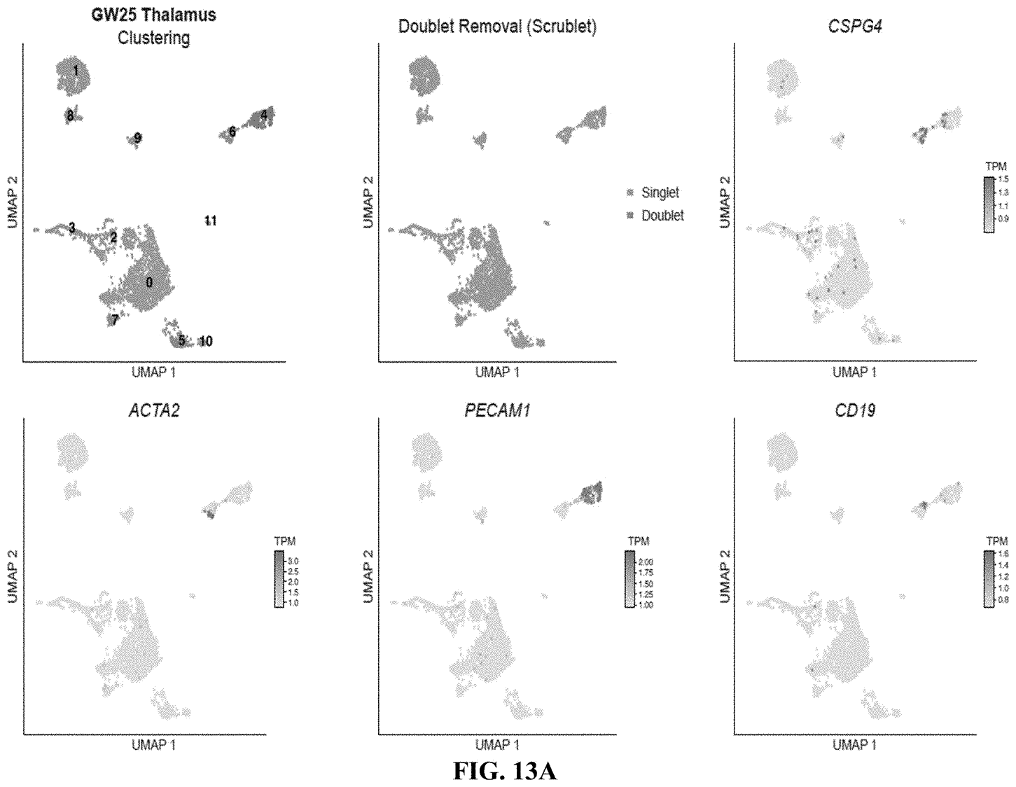

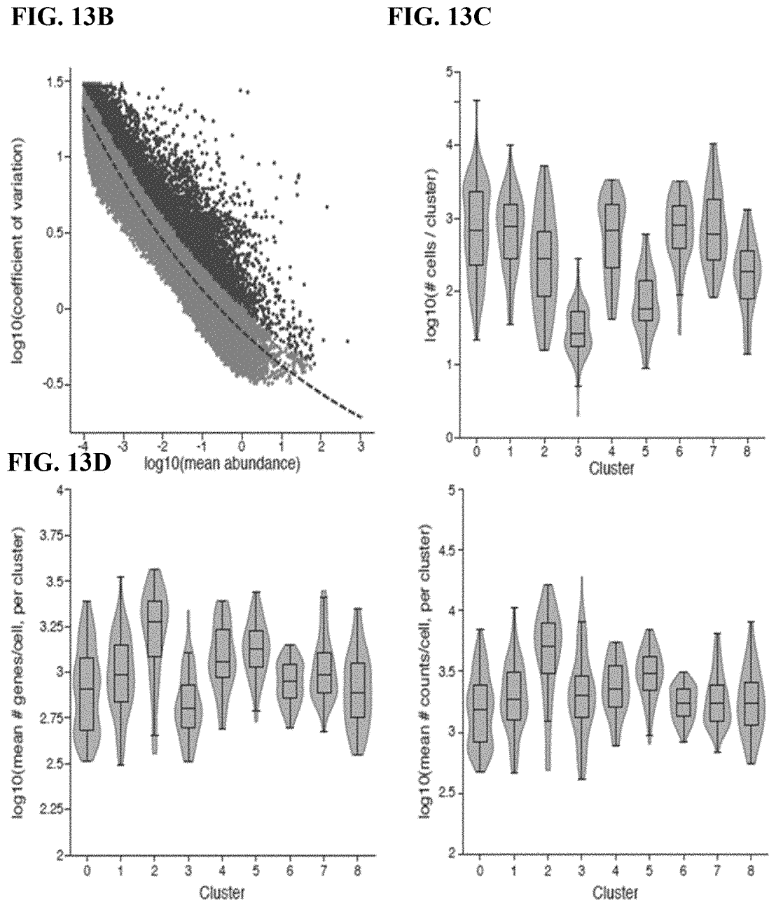

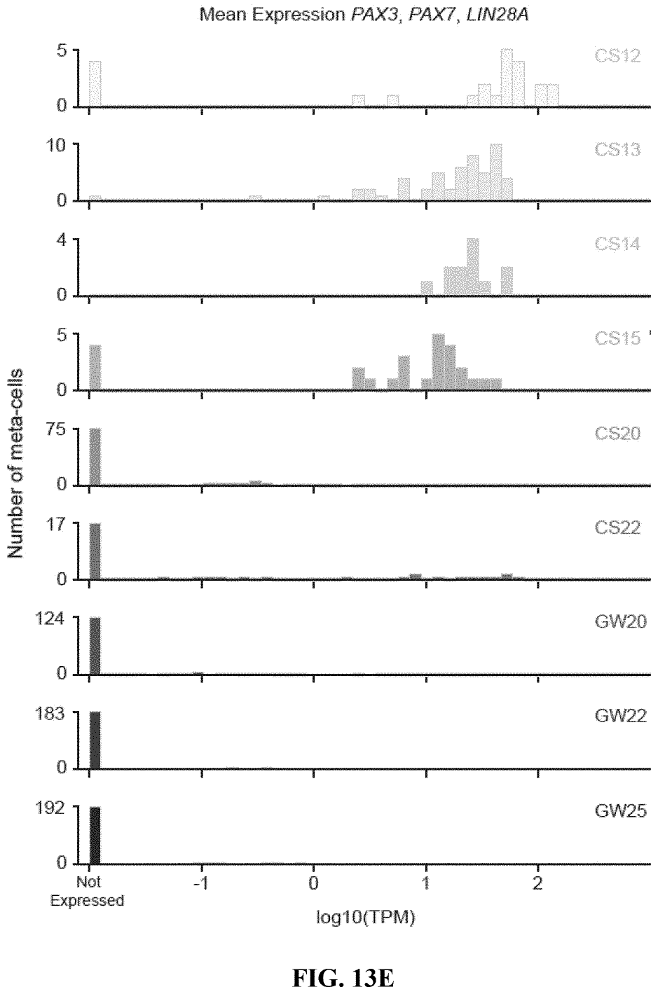

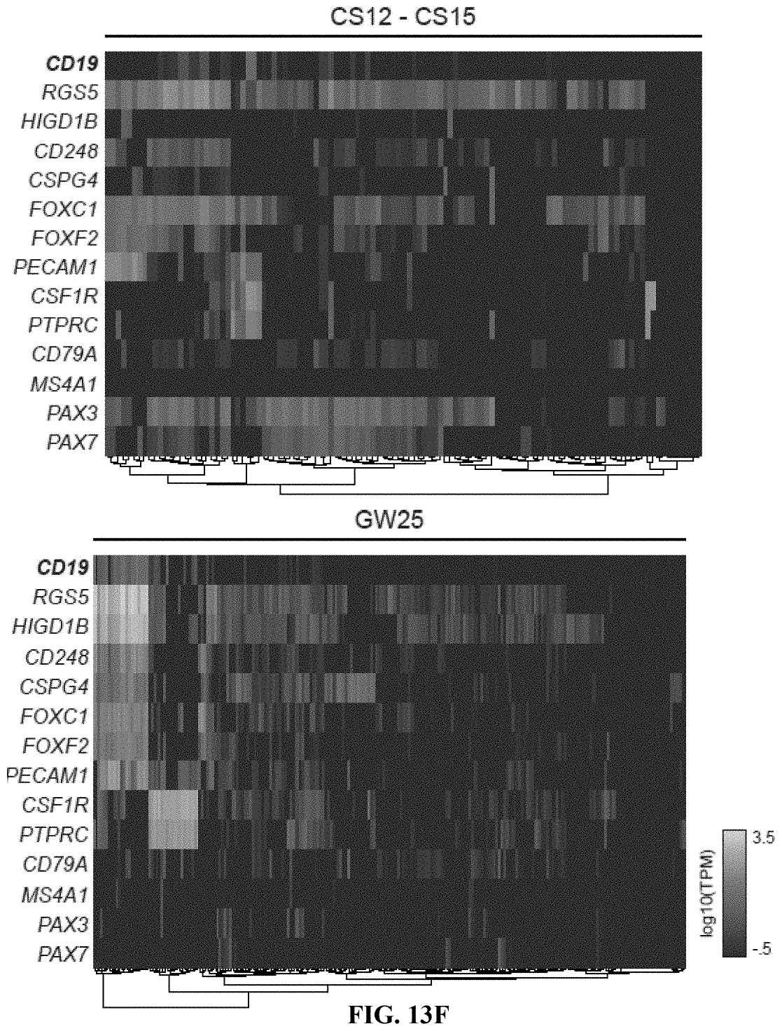

Analysis of Kriegstein/BICCN meta-cells: To identify meta-cells in the data from the Kriegstein lab/BICCN data, the processed UMI counts were first analyzed using Scrublet on a per-sample basis to identify potential doublets. The following parameters were used: expected_doublet_ratio=0.08, sim_doublet_ratio=2, min_counts=3, min_cells=3, min_gene_variability_pct1=75, n_prin_comps=30. A consistent threshold for doublet calling was set at 0.35 across all samples. The results of this were then saved and imported alongside the UMI counts into Seurat. Samples were all processed individually with the same pipeline, using the same parameters and clustering resolution. As a result, some samples may be under-clustered, as the clustering resolution was intentionally set to err on the side of merging similar cells rather than creating false positive clusters. Only cells with >250 and <4000 features were included, as well as less than 20% mitochondrial reads. A cell cycle difference score was calculated following the Seurat vignette. The SCTransform workflow was used, and percent mitochondrial reads, number of genes detected, and cell cycle difference were included as variables to regress out. 30 components were used for UMAR dimensionality reduction, with a random seed of 1, and n.neighbors=100. 30 components were used for FindNeighbors, and a resolution of 0.2 was used for FindClusters. Subsequently, predicted doublets were excluded, and the average depth-normalized expression of each cluster was calculated using the AverageExpression function. This information, representing the meta-cell transcriptome, alongside the individual cell metadata (e.g., cell barcode, cluster ID, UMAR coordinates, etc.), and the meta-cell metadata (e.g., number of cells per metacell, mean number of genes per cell for each meta-cell, etc.) was saved for each sample. A representative example of this output is shown in FIG. 13A.

This information was then imported into Python and merged, to create a metacell x genes matrix, with each value representing mean(depth-normalized UMI counts). Variable genes were identified by fitting an exponential function to the log(mean) versus log(coefficient of variation) for each gene across meta-cells. The top 6000 genes, representing −20%, based on distance from the fit line, were used for PCA. Data was log transformed, then z-normalized, prior to PCA. The scikit-learn decomposition.PCA( ) function was used with the svd_solver=‘arpack’. Then, the top PCs were used as input to the UMAR reducer in the umap-learn package with the following parameters: n_neighbors=80, n_components=2, min_dist=0.25, transform_seed=50, n_epochs=500. Last, the top 30 PCs were used as input to KMeans clustering in the scikit-learn package, with k=9.

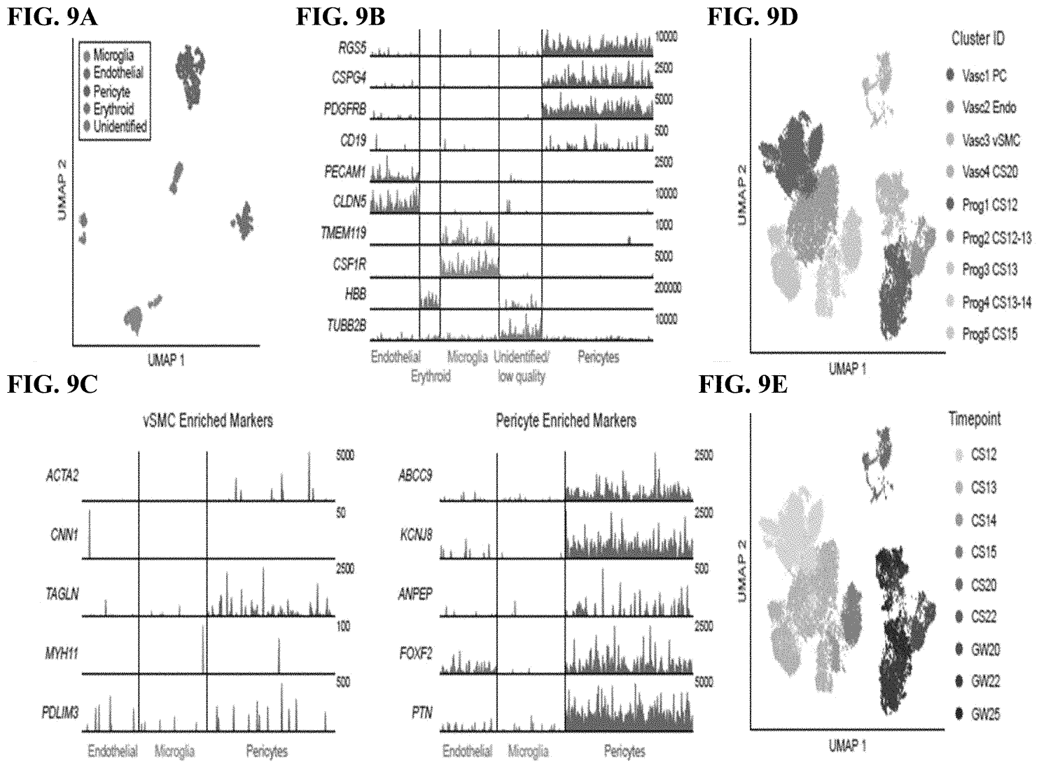

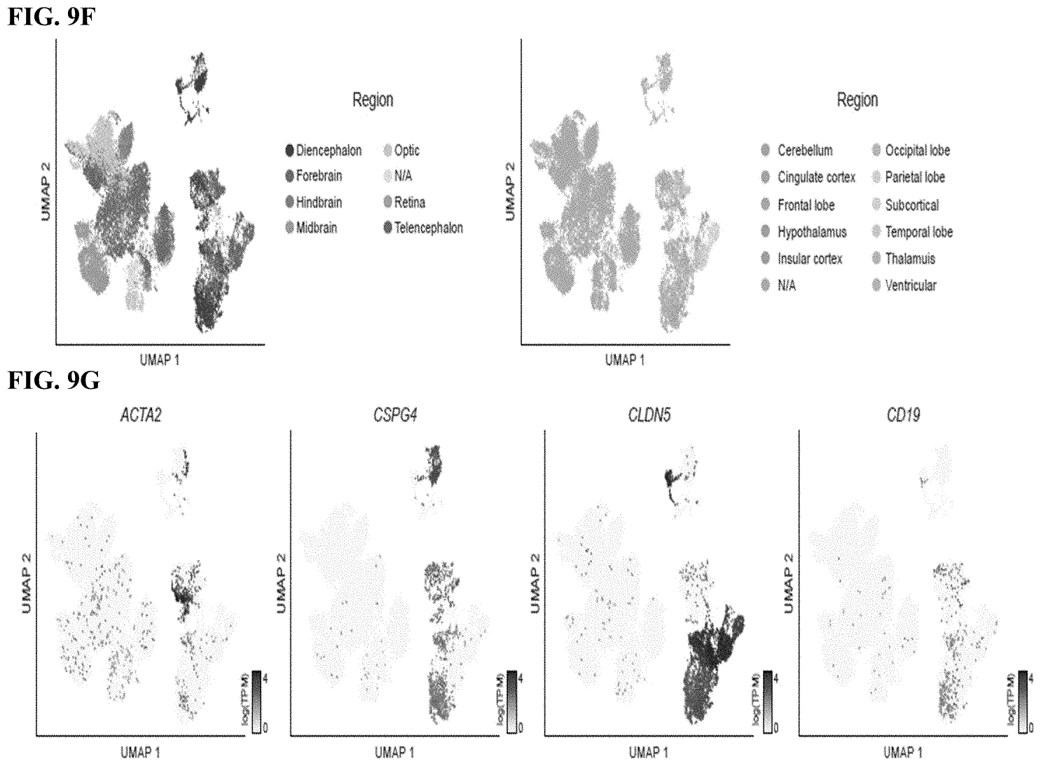

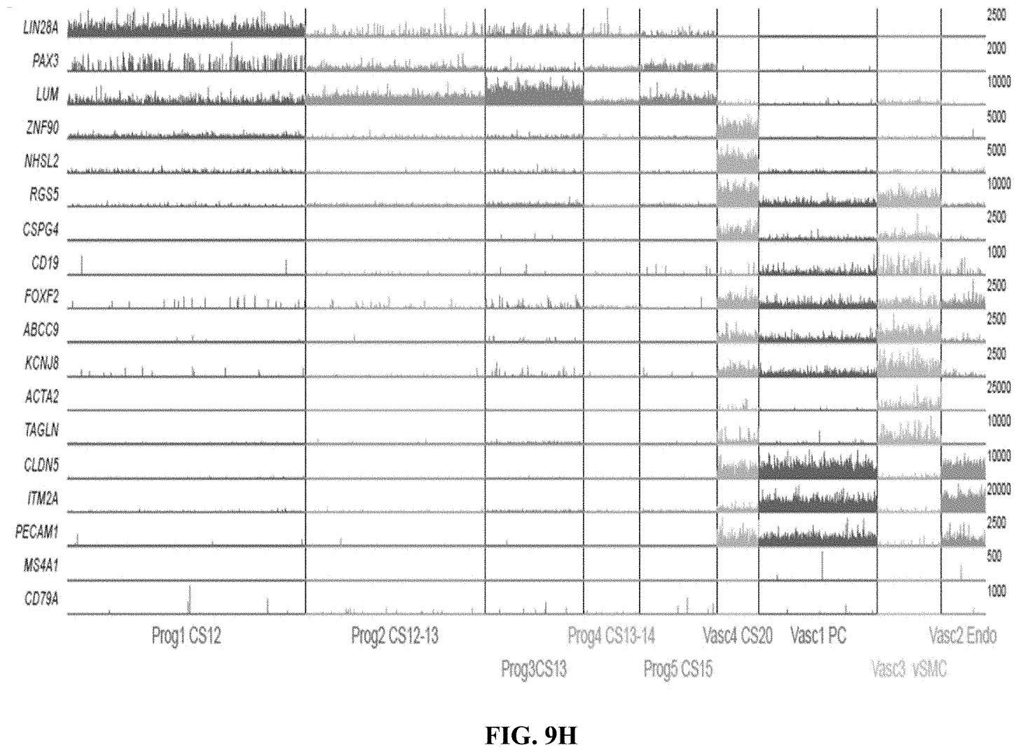

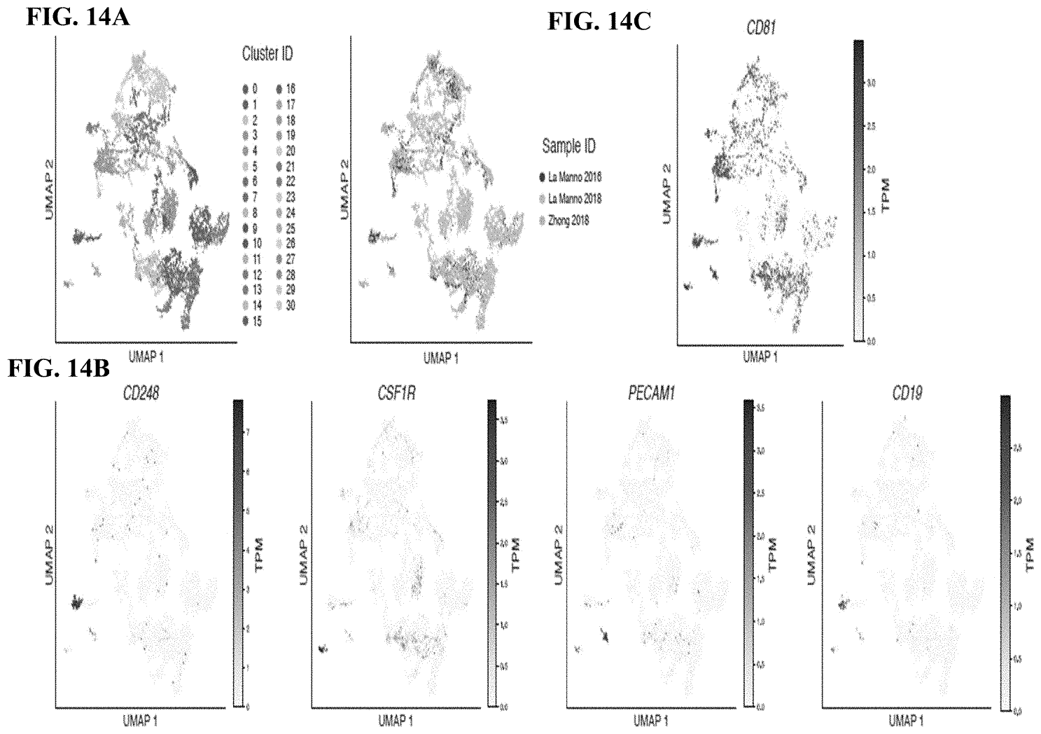

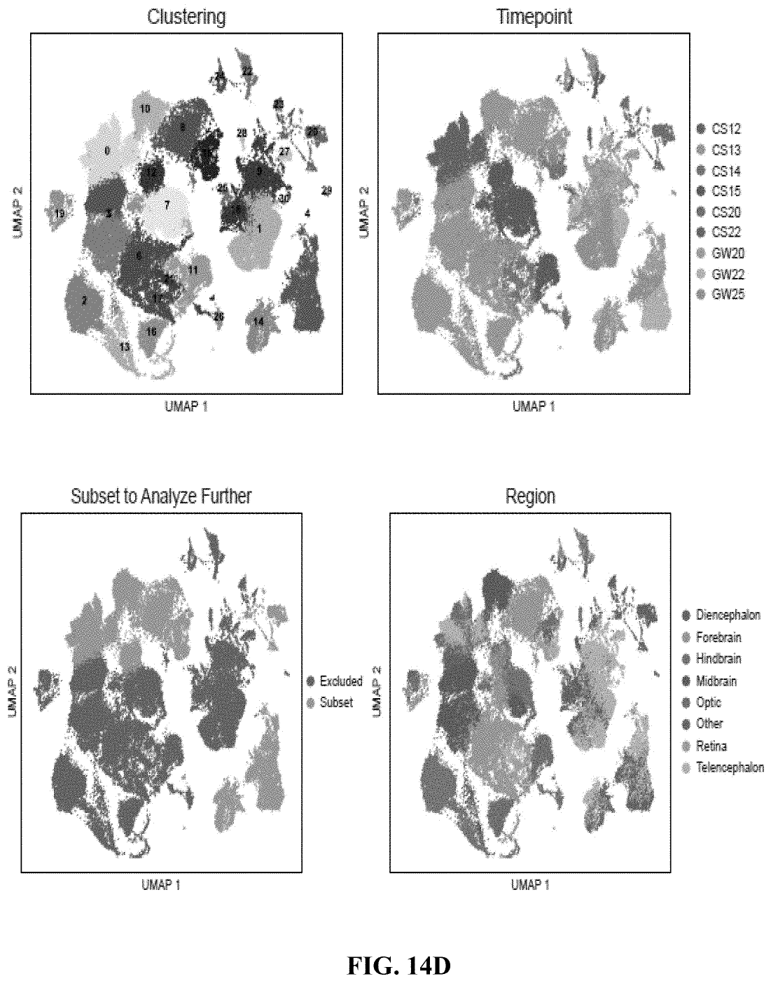

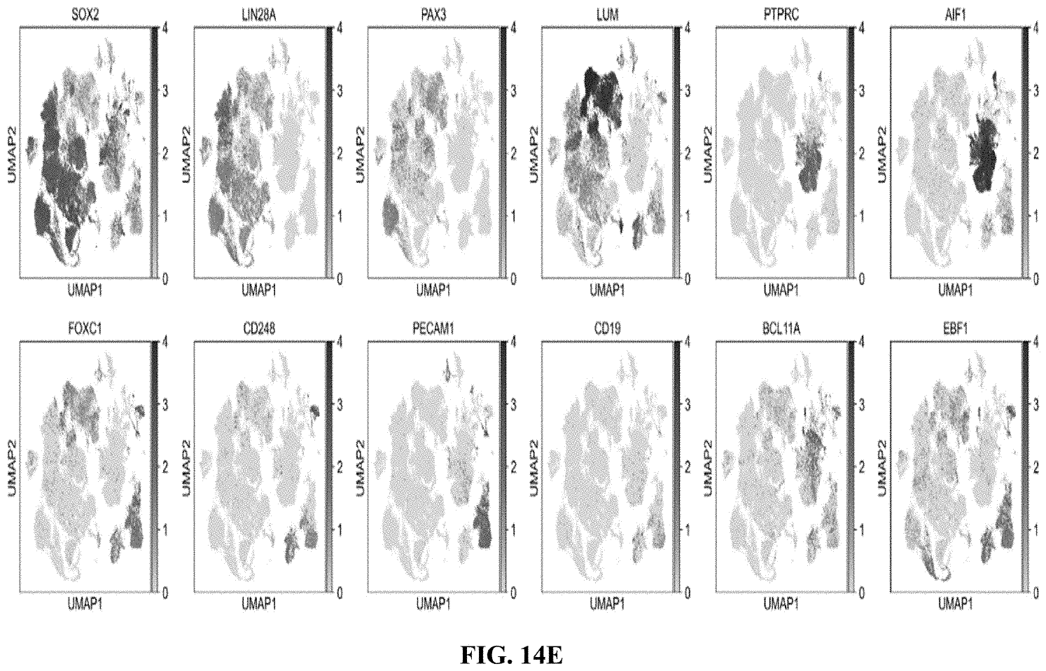

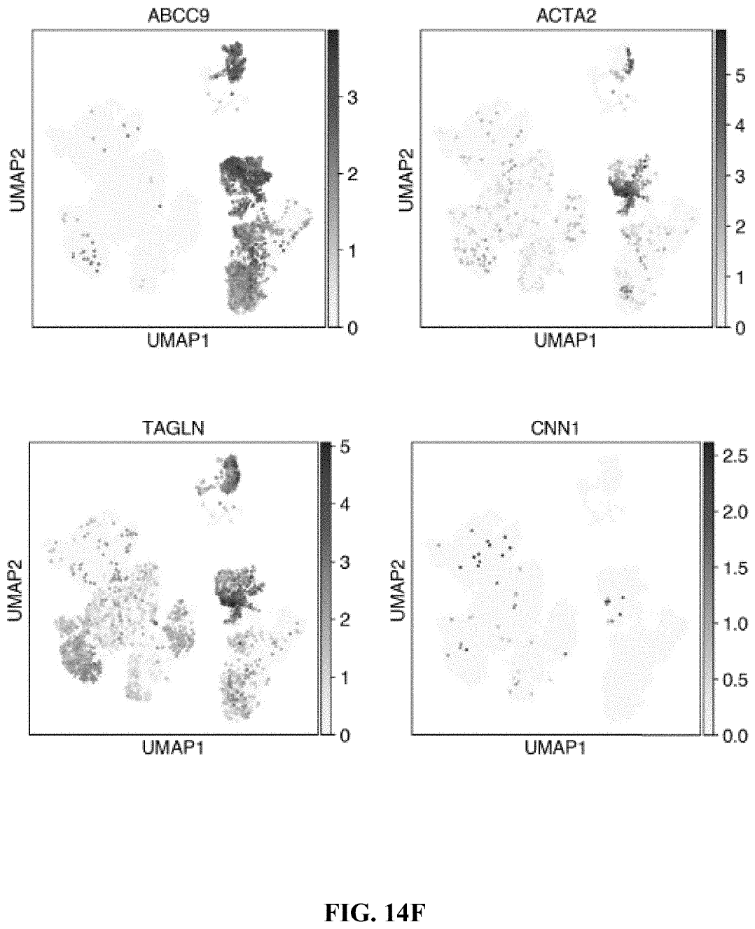

Analysis of vSMC vs pericyte identity: To generate the integrated dataset shown in FIGS. 9A-9C and 14A-14C, the data from Zhong 2018, La Manno 2016, and La Manno 2018 was first processed using Scrublet to identify doublets with the following paramters: expected_doublet_rate=0.06, min_counts=2, min_cells=3, min_gene_variability_pct1=85, n_prin_comps=30. A threshold of 0.25 was used for doublet calling. The doublet information along with expression information was then imported into Seurat. Individual Seurat objects were created, using only the union of genes annotated in all three datasets (to account for small differences in upstream sample processing), and only including cells with at least 500 features detected. Cell cycle scoring was performed as described above, and each sample was processed using SCTransform withvars.to.regress=nFeature_RNA and percent.mt. 3000 features for integration were then chosen using the SelectlntegrationFeatures function, followed by PrepSCTIntegration, FindlntegrationAnchors, and IntegrateData using default parameters. For RunUMAP, the following parameters were used: dims=1:30, seed.use=20, min.dist=0.3, spread=1. 50 PCs were used as input for FindNeighbors and resolution=1 was used for FindClusters. This output is shown in FIG. 14A.

The subset of cells that were in clusters expressing pericyte/vSMCgenes (RGS5, CSPG4, FOXF2, ACTA2), endothelial genes (PECAMI) or microglial genes (CSF1R) were included for downstream analysis. Predicted doublets were excluded from the cells falling into these clusters. These samples were re-processed using the same paramters for RunUMAP, FindNeighbors, and FindClusters as above, with the exception of dims=1:30 for FindNeighbors and resolutions for FindClusters.

To generate the dataset shown in FIGS. 9D-9H and 14D-14F, meta-cells from cluster ID=3, 4, 6, or 9 were included. The 92K single cells that comprised these meta-cells were identified and imported into Scanpy. These were first processed, excluding cells with fewer than 500 genes, genes detected in fewer than 3 cells, and cells with more than 10% mitochondrial reads. Doublets had already been excluded in the generation of meta-cells, and so all cells analyzed here were not predicted doublets. Variable genes were identified with min_mean=0.0125, max_mean=3, min-dist=0.5. n_counts was regressed out, and max_value=10 was used for pp.scaleQ. 50 PCs were calculated and used for the pp.neighbors( ) calculation; default parameters were used fortl.umap( ) and resolutions.65 was used for tl.leiden( ). This output is shown in FIG. 14D.

Then, clusters identified as pericytes/vSMCs/endothelial cells, as well as the subset of progenitor (early timepoints) cells that were SOX2 low and showed scattered expression of pericyte markers were analyzed. This comprised 26K cells. These samples were reprocessed using the same workflow, unless noted here: maximum percent_mito=8%, resolution=0.25 for tl.leiden( ).

Analysis of Brainspan data: RPKM data processed at the gene- and exon-level was downloaded from the Allen Institute portal. For correlation analysis, spearman correlation was used. The top 200 genes by spearman correlation were analyzed using the PANTHER web tool. PBMC data was integrated with the data from the Zhong 2018, La Manno 2016, and La Manno 2018 datasets. The BICCN data was not included due to the less clear separation between pericytes and endothelial cells observed in this data, presumably due to incomplete dissociation of the tightly interacting cell types leading to a high rate of false positive endothelial transcripts being present in pericyte cells and vice-versa. These samples were merged and processed using Scanpy using join=outer. Cells with greater than 20% mitochondrial genes were excluded. To calculate a gene score, the top 30 genes identified based on spearman correlation in the Brainspan data were used as inputs to the tl.score_genes( ) function in Scanpy, with the following parameters: ctrl_size=50, random_state=1, n_bins=10. The ttest_ind( ) function inscipy.stats was used to calculate p-values for gene scores between the different clusters.

Analysis of lung data: Lung data was first processed using Seurat to identify clusters with high expression of pericyte and endothelial marker genes. These clusters, which included ˜19K cells, were then merged with the brain data from Zhong 2018, La Manno 2016, and La Manno 2018. The BICCN data was excluded for the same reason as above. These cells were then processed together to allow identification of the large vSMC, pericyte, and endothelial cell clusters in the lung data. The annotations of pericyte/endothelial cell identity were carried over from the above merged analysis of the three datasets described in the analysis of vSMC vs. pericyte identity of these datasets (FIGS. 9A-9C). Diffxpy was used to perform differential gene expression testing between the different pairwise comparisons of interest (e.g., brain vs. lung pericytes). Due to the vast difference in the number of lung pericytes and endothelial cells versus brain cells, the lung data was sub-sampled to match the same number of cells in the respective brain cluster being analyzed in order to avoid confounding effects due to differences in cell number (i.e., the lung data had greater power to detect lowly-expressed genes).

Generation of mouse single-cell RNA-sequencing data: Four mouse brains were dissected and dissociated following an established protocol. Briefly, whole brain was minced using a razor blade, and all tissue (not following the note to remove vessels as in the protocol) was pooled and transferred into a tube filled with an enzymatic dissociation solution containing 250 ug/mL STEMxyme I. This was incubated at 37 degrees for 30 minutes, then triturated, and filtered using a sucrose gradient to remove myelin debris. Red blood cell lysis was performed with ACK lysis buffer. The resulting cells were then resuspended in FACS buffer (HBSS without Ca/Mg, supplemented with 1% BSA), blocked at RT for 10 minutes using BioLegend TruStaing FcX (#101320), then stained for 20 minutes on ice with the following antibodies: CD19 (BioLegend 115512, diluted 1:100), CD13 (BD 558745, diluted 1:20), CD31 (BioLegend 102421, diluted 1:50), and CD45 (BioLegend 103113, diluted 1:100). CD19+, CD31+, and CD13+ cells were enriched by flow sorting prior to capture using the 10× Genomics 5′ scRNA kit following the manufacturer protocol. Libraries were sequenced using a HiSeq 2500.

Analysis of mouse single-cell RNA-sequencing data: Data was processed using CellRanger v3.0.2 using the prebuilt mm10 index vS.O.O. The resulting files were then processed using Scrub let to remove suspected doublets with the following settings: miin_counts=2, min_cells=3, min_gene_variability_pct1=85, n_prin_comps=30, threshold=0.25. Doublets were then excluded, and the remaining data was then processed using Scanpy, excluding cells with less than 250 genes or more than 10% mitochondrial reads. Variable genes were selected with the following parameters: min_mean=0.0125, max_mean=3, min_disp=0.5. PCs were used for pp.neighbors( ), and data was clustered using tl.leiden( ) with resolution=0.15.

Flow cytometry: For the mouse CNS neurovascular fraction characterization experiments, the following antibodies against mouse molecules were used: anti-mouse CD19 (clone 6D5 0.1 mg/ml, BV650, Biolegend, dilution 1 to 200), CD45 (clone 30-F11, APC-Fire750 Biolegend, dilution 1 to 100), CD31 (clone 390, PerCP-Cy5.5, Biolegend, dilution 1 to 400), B220 (clone RA3-6B2, PE/Dazzle 594 Biolegend, dilution 1 to 200), Zombie Aqua Fixable Viaiblity kit (dilution 1 to 400). Isolation and flow cytometric analysis of CNS perivascular cells was performed according to Crouch et. al, with modifications to digestion volumes to accommodate whole murine brain rather than micro regions. Antibody staining was done using CD13-PE (BD Biosciences, cat. No. 558745), CD31-PECy7 (BD Biosciences, clone 390, cat no. 102418), CD41-APC (Biolegend, cat. no. 133914), CD45-APC (Biolegend, clone 30-F11, cat no. 103112), CD19-BV421 (BD Biosciences, clone 1D3, cat. no. 562701), and BV421 Rat lgG2a k isotype control (BD Biosciences, cat. no. 400549). Dead cells were excluded with 7-AAD (Biolegend, 420404). Fluorescence minus one (FMO) controls were used for each maker to define gates for each population. Samples were acquired on a BD 5-laser LSRFortessa or 4-laser BD Aria II. Note: epitope cleavage of CD13 and CD19 and loss of cell viability were observed with harsher digestion protocols using papain or higher concentrations of collagenase/dispase. Species-matched isotype controls were performed for each primary antibody to ensure the absence of non-specific binding. A fluorophore minus one (FMO) control was also used for each individual marker staining, to define gates for each population. An important challenge for these perivascular cell suspension preparations is the cell stress during dissociation due to trituration and digestion by enzymes, both known to impact cell viability and cause the low cell numbers we observed in our experiments. The dissociation itself is also known to cause significant cell surface marker epitope cleavage. CD13 and CD19 expression was present but variable according to the enzyme used (papain, collagenase, or hyaluronidase) for digestion.

Human T cell viability was determined by staining with LIVE/DEAD Fixable Violet Dead Cell Stain Kit (Invitrogen), followed by surface antibody staining. CAR expression was detected using biotinylated protein L (Genscript) followed by R-phycoerythrin-conjugated streptavidin (BD cat. #554061). All experiments were acquired on a BD LSR Fortessa flow cytometer (Becton Dickinson) and data was analyzed with FlowJo v10.

Cytotoxicity assays: All cytotoxicity assays were flow cytometry-based and adapted from the Quah et al. protocol (Quah et al., 2007). Target cells were stained with CFSE (Thermo Fisher) and T cell effectors were stained with Cell Trace Far Red (Thermo Fisher) prior to co-culture. 24 hours after co-culture, cells were harvested, stained for viability with LIVE/DEAD Fixable Violet Dead Cell Stain Kit and analyzed by flow cytometry.

BBB permeability assays: Evans Blue dye (EBD) infusion was performed as previously described (Radu and Chernoff, 2013). Seven days after CAR-T cell infusion, mice were injected intravenously with EBD+/−mannitol. Thirty minutes after EBD infusion, mice were euthanized; brain tissues were excised and fixed in 10% formalin solution for at least 24 h. After washing and dehydrating, brains were embedded in paraffin and cross-sectioned at a thickness of 5 pm. Sections were deparaffinized, mounted with DAPI-containing Vectashield, and analyzed by fluorescence microscopy.

C57BL/6J mice were obtained from Jackson Laboratory and housed in a BSL1 mouse facility at the University of Pennsylvania. Murine splenocytes from donor mice were harvested, activated with murine-specific anti-CD3/anti-CD28 magnetic beads (Thermo Fisher), and transduced with retroviral vector encoding mCD19mBBz CAR, mCD19mCD28z CAR, or hCD19mBBz CAR transgenes. Four days after transduction, 5×10⁶ murine CAR-T cells were injected intravenously in recipient mice+/−(50 mg/kg cyclophosphamide for lymphodepletion.

Immunohistochemistry: The human anti-CD19 antibody (clone BT51E) was used to stain normal human brain. Images were visualized and acquired using the Leica #NCL-L-CD19-163 microscope. Automated immunohistochemistry was performed with Ventana Benchmark XT following a clinically validated protocol for CD19. 4-micron FFPE tissue sections were deparaffinized and rehydrated. Antigen retrieval wasperformed using Standard Cell Condition 1 (pH 8.5) for 60 minutes (Ventana Medical Systems). Slides were incubated with anti-human CD19 (1:50, monoclonal, Abnova BT51E) at 370 for 30 minutes. The ultraView Universal DAB Detection system (Ventana) was used with 3,3′diaminobenzidine chromogen.

High resolution in vivo brain MRI: 3×10⁷ T cells with 32% normalized CAR+ expression were counted using the ADAM-CellT (NanoEnTek, Seoul, Korea) and the Multisizer 4e (Beckman Coulter). T cells were administered to non-tumor bearing NSG mice (n=4 per group) by one intravenous tail vein injection in 100 μL of PBS. All the MR imaging experiments were performed 4 days post CAR-T cell infusions. Mice were anesthetized by using isoflurane maintained at 1-1.5% in 1 liter/min air. Lateral tail vein was cannulated for administration of gadodiamide (287 mg/ml) or mannitol (25% w/v). Mice were placed in a 20 mm diameter commercial quadrature proton coil (m2m Imaging Corp., Cleveland, Ohio) and the probe was transferred into a 9.4T horizontal bore small animal MR scanner (Bruker, Billerica, Mass.). During the MRI scans, animal body temperature was maintained at 37° C. with the air generated and blowing through a heater (SA Instruments, Inc., Stony Brook, N.Y.). Respiration and body temperature were continuously monitored using an MRI compatible small animal monitoring system (SA Instruments, Inc., Stony Brook, N.Y.).

T1-mapping was performed pre- and post-gadodiamide injection on 0.7 mm thick coronal slice in the mid-brain using saturation recovery protocol with following parameters, number of averages=2, field of view=20×20 mm², matrix size=192×192, echo time=8 ms, repetition times=200, 500, 800, 1200, 1500, 4000 and 9000 ms, scan time 30 minutes. Following the baseline T1 acquisition, gadodia mide was administered via tail vein as described previously (Ku et al., 2018, Methods Mol. Biol. Clifton N.J. 1718, 395-408.; van Vliet et al., 2014, Neurobiol. Dis. 63, 74-84). T1 acquisition scans were run consecutively after one another. Pre- and Post-gadodiamide administration T1 maps were generated using the image sequence analysis tool in Paravision 6.0.1 by exponential fitting of the signal recovery. An in-house MATLAB script was usedtoquantity the ATl (T1 Post⁻ T1 Pre) caused by Gd leakage in the brain.

Quantification and Statistical Analysis: Information on specific statistical tests used is provided in the figure legends and/or Method Details. Quantification of MRI ATl was performed using a MATLAB script. For single-cell RNA-sequencing analysis, specific details regarding pre-processing steps and parameters are provided in the relevant section of the materials and methods.

The results of the experiments are now described.

### Example 5: CD19-Positive Mural Cells Identified by scRNA-Sequencing in Human Brain

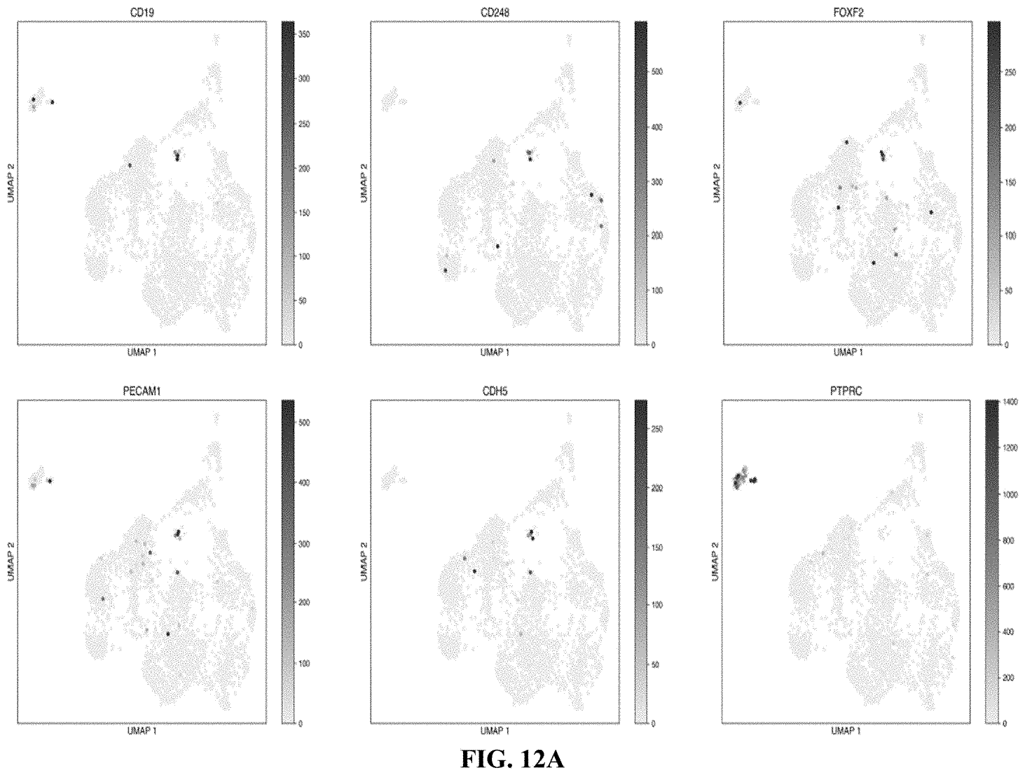

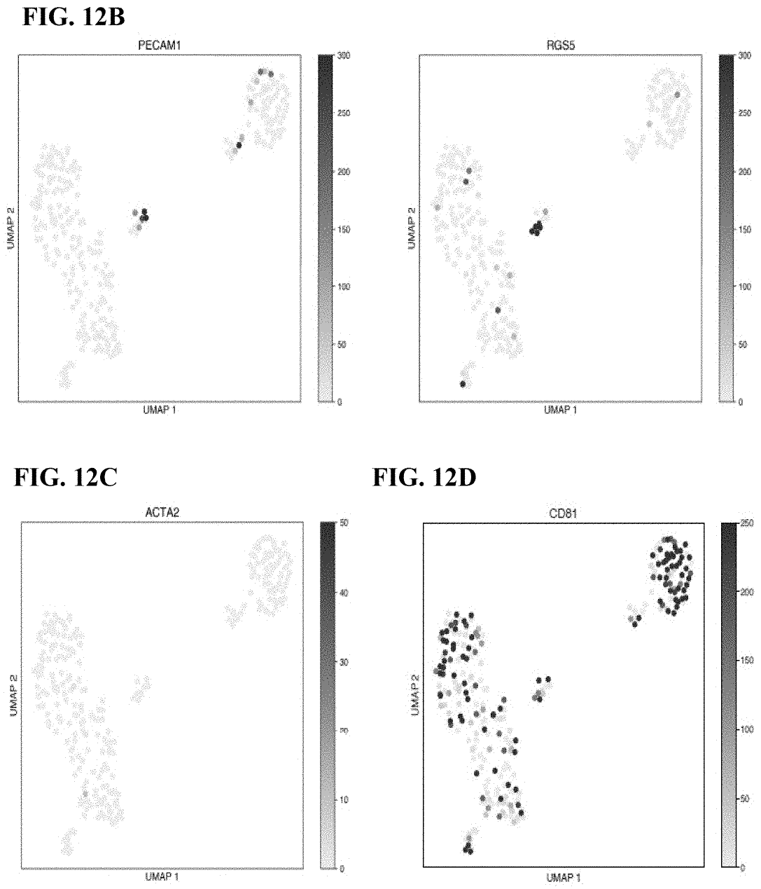

It was hypothesized that if a population of CD19-expressing cells is present in the human brain, it might be identifiable in data generated from recent efforts to map the human brain transcriptome with single-cell resolution. ScRNA-seq data was analyzed from 2,364 human prefrontal cortex cells (Zhong et al., 2018, Nature 555, 524-528; FIG. 1A). Cells were clustered and broad populations identified, focusing subsequent analyses on non-neuronal, non-erythroid cells. These further segregated into astrocyte, lymphocyte, microglial, oligodendrocyte precursor, endothelial, and pericyte populations (FIGS. 1B, 1C, and 12A). These populations were identified based on the expression canonical marker genes; mural cells specifically expressed expected marker genes, such as PDGFRB, FOXF2, RGS5, and CD248, while endothelial cells expressed a distinct set of markers, including CDH5 and PECAM1 (CD31; FIG. 1D). Surprisingly, this analysis revealed a small population of cells (−1.5% of non-neuronal cells; −0.2% including neuronal cells) that expressed CD19 and co-expressed the mural cell marker CD248 (FIGS. 1E and 12A). These cells were negative for the vSMC marker ACTA2, indicating that these cells were pericytes (FIG. 12C). Importantly, this population was negative for the B cell marker CD79A, arguing against the possibility of artifactual CD19 expression due to B cell pericyte doublets (FIG. 1E). Additionally, these cells were positive for the tetraspanin CD81, which chaperones CD19 through secretory pathways to the plasma membrane and is required for surface expression of CD19 in B cells (FIG. 12D). A separate population of lymphocytes was observed that expressed the marker genes CD45 (PTPRC), CD3, CD7, and IL2RG (CD132), representing a population of T cells (FIGS. 1E-1F). Notably, the expression of lymphocyte markers and pericyte markers was mutually exclusive in the observed clusters, and the expression of CD19 was specific to the pericyte cluster (FIG. 1F).

Due to the inherent sparsity of single-cell data, even highly expressed genes will not be detected in every cell, causing single-cell data to be zero-inflated. To ask at what level CD19 is expressed in pericytes, the expression of CD19 was compared to known pericyte marker genes, relative to the overall distribution of gene expression in pericytes. Mean-normalized gene expression values can provide a rough estimate of the relative expression of a given gene in a cell population of interest, despite the inherent sparsity of single-cell data. As expected, pericyte markers such as CD248, RGS5, and PDGFRB ranked in the top percentiles of gene expression in pericytes from the human prefrontal cortex (85^{th}, 96^{th}, and 98^{th}, respectively) (FIG. 1G). CD19 was similarly expressed highly, in the 86^{th} percentile for gene expression.

This observation was reproduced in additional independent scRNA-seq datasets. In a dataset from human forebrain (La Manno et al., 2018, Nature, 560, 494-498), a mural cell population (48/7906 cells) positive for the markers CD248 and RGS5 also displayed CD19 expression (12/48 cells had measurable 0019) and the absence of the B-cell marker CD79A (FIG. 7A). In a third dataset derived from human ventral forebrain (La Manno et al., 2016, Cell 167, 566-580.e19), expression of CD19 was again observed in a population of CD248 positive mural cells (195 mural cells/1977 cells, of which 25/195 had measurable CD19 (FIG. 7B). As with other datasets, no RNA-sequencing counts for CD79A were identified in any mural cells. Although spurious non-mural CD79-positive cells were observable, these were likely the result of cell-cell doublets, such as mural-endothelial cell doublets observed due to the adhesion of mural to endothelial cells in the brain. In these datasets, CD19 ranked in the 86^{th} and 71^{st} percentiles for gene expression, suggesting relatively robust expression (FIG. 7C).

### Example 6: CD19 Expression in Adult Human Brain by IHC

To assess the expression of CD19 protein in human mural cells, immunohistochemistry was performed on several regions of the human brain using a clinically validated anti-human CD19 antibody (clone BT51E) on samples from healthy deceased subjects. This antibody recognizes the C-terminus of the CD19 protein, which is cytoplasmically localized. CD19 expression was found on cells present adjacent to the vessel basement membrane walls in perivascular areas (FIG. 2A-2C). Abluminal CD19 expression was observed across multiple brain regions, with particular regions, such as the hippocampus, insula, temporal lobe, frontal lobe, and parietal lobe displaying a comparatively higher, albeit still rare, incidence of CD19-positive cells. In contrast, regions such as the pons and occipital lobe displayed lower rates of CD 19-positive cells (FIG. 2A-2C). Notably, CD19 positive cells were found along smaller capillaries (<8 pm) as well as larger vessels (>8 pm; majority of cells depicted), suggesting that in addition to pericytes, CD19 may also be expressed in vSMCs. It is possible that the CD19 staining observed by IHC may also be due to other cell types, such as passing B cells or glia. However, the abluminal localization along the vasculature of CD19+ cells is most consistent with staining of mural cells.

### Example 7: Analysis of Pericytes and vSMCs Shows CD19 Expression Across Mural Cells

Based on the perivascular staining of CD19 along both larger and smaller vessels, which suggested that CD19 expression might be a more general feature of mural cells, the transcriptome of pericytes and vSMCs was analyzed. A large scRNA-seq dataset generated by the BRAIN Initiative Cell Census Network (BICCN, Kriegstein/UCSF), comprising scRNA-seq data from diverse human brain regions over many developmental timepoints spanning Carnegie Stage 12 (CS12), corresponding to gestational week (GW) 4, through GW25 was analyzed. This dataset included roughly 857,000 cells across 101 individual samples, affording us the power to perform a detailed analysis of the cell types within the NVU.

All 101 samples were first processed individually, using the same pipeline, removing any cells identified as putative doublets and then identifying distinct clusters of transcriptionally similar cells (FIGS. 8A, 13A). These clusters were aggregated into “meta-cell” transcriptomes, representing the average gene expression across the cells in that cluster, allowing efficient identification of populations of interest within the larger dataset. Together, this analysis identified 855 meta-cells, which were then analyzed together. Highly variable genes were identified across the 855 meta-cells, then these genes were used to perform dimensionality reduction and clustering (FIGS. 8B, 13B). Importantly, the clusters of meta-cells were largely similar on quality measures, such as the number of single cells per meta-cell, and mean number of counts per meta-cell (FIG. 13C).

This analysis revealed that CD19 is highly expressed in neurovascular meta-cell clusters, consistent with our prior findings (FIGS. 8C-8D). The meta-cell analysis does not show a clear separation between mural and endothelial cells (or other NVU cell types), since meta-cells may include closely clustering cell types in sparse individual samples. However, this clustering shows clear separation of meta-cells expressing mural markers, and the expression of CD19 and absence of B cell markers in these meta-cells (FIGS. 8E-8F). Additionally, a strong correlation was observed between expression of mural cell marker genes and CD19 across the data (FIG. 8G). Low-level CD19 expression was also observed in microglia clusters (FIG. 8G-8H). However, as mural cells and other cells of the NVU, which include perivascular macrophages, are tightly connected, this expression is likely the result of contamination from mural cells in microglia meta-cells.

Notably, mural meta-cells were not present in early timepoints (CS12-CS15), which had high expression of early developmental markers such as PAX3 and LIN28A (FIG. 8D, 13E). In contrast, mural meta-cells at GW25 displayed high and specific expression of mural cell marker genes, such as CD248, RGS5, and FOXF2, in addition to specific expression of CD19 (FIG. 13F). In this data, undifferentiated progenitors were clearly separated from differentiated mural cells, which demonstrates that once mural cells emerge, they express CD19 (FIG. 8E-F, 13E-F). Collectively, there was clear separation of meta-cells expressing neurovascular markers from all other meta-cells (FIG. 8I), which allowed identification of cells of interest for further analysis.

Pericytes are closely related to vSMCs, which together are the mural cells that line the brain vasculature. These cells differ based on anatomical position; pericytes localize around capillaries and vSMCs localize around larger vessels, including arteries, arterioles, and venules. However, these cells are transcriptionally similar, sharing the identity of many marker genes and appearing to exist on a transcriptional lineage continuum. ACTA2, encoding alpha-smooth muscle actin, is a canonical marker used to distinguish these two populations, which is significantly upregulated in vSMCs. Many pericyte markers, however, such as CSPG4 and RGS5, are also highly expressed in vSMCs, causing brain vSMCs to often be annotated as pericytes.

It was first asked whether the cells originally identified as CD19+ mural cells represented pericytes or vSMCs. The three datasets from Zhong, 2018, La Manno, 2016, and La Manno, 2018, were aggregated and analyzed as a single integrated dataset (FIG. 14A). A subset of non-neuronal clusters enriched for mural marker genes (CD248, CSPG4) were identified, and it was confirmed that this population expressed both CD19 and CD81 (FIGS. 14B-14C). Subsets expressing the endothelial and microglial markers, CD248 and CSFIR, respectively, were also identified (FIG. 14B). The non-neuronal subset (mural cells, endothelial cells, and microglia) was then re-analyzed and clustered to distinguish between transcriptional differences in cell types of the NVU (FIGS. 9A-9B). These cells showed strong enrichment of reported pericyte markers, such as ABCC9 and KCNJ8, without enrichment of ACTA2 or other vSMC marker genes, suggesting that they represented bona-fide pericytes (FIG. 9C).

This analysis was repeated using the cells from the BICCN dataset, performing a detailed analysis of neurovascular cells and related progenitors. Using the meta-cells as a guide, ˜92K single cells representing the NVU as well as early progenitors were identified (FIGS. 8B, 14D). A bifurcation of progenitors from the early timepoints that appeared to be neural lineage-biased, and others that seemed biased toward non-neuronal lineages was observed. The non-neuronal-biased progenitors, as well as the pericyte and endothelial cell clusters were then subset for further analysis (FIG. 14E). These ˜26K cells showed a clear separation between progenitors from CS12-CS15) and differentiated NVU cells from GW20-GW25 (FIGS. 9D-9E). They were also representative of cells from many brain regions (FIG. 9F). Note that samples were annotated at different levels of granularity, such as forebrain versus diencephalon or telencephalon; and only later samples had finer-grained regional resolution. Notably, a smaller vSMC population was identified in this dataset, likely due to the high number of cells in the BICCN data, as pericytes were more abundant than vSMCs (FIG. 9G). These cells showed high expression of ACTA2 and TAGLN, as well as other pericyte markers such as RGS5 (FIG. 9H). Importantly, CD19 was expressed in the vSMC population as well, consistent with the high transcriptional similarity between these two populations. This suggests that CD19 expression is not unique to pericytes, but a common feature in human brain mural cells, consistent with the staining pattern observed by IHC (FIGS. 2A-2C). Further assigning the identity of this vSMC population to venule, arterial, and arteriole sub-populations was challenging since canonical markers identified in mice did not perfectly align with the sub-populations identified in this human dataset, suggesting transcriptional differences between mouse and human mural cells (FIG. 14F).

Within the BICCN dataset, a higher degree of endothelial marker gene expression was observed in mural cell clusters, and vice-versa. For example, while endothelial and mural cell clusters are distinguishable, the expression of CLDN5, and endothelial cell marker, and CSPG4, a mural cell marker, is more overlapping than would be expected (FIG. 9G). It was postulated that this is likely due to incomplete separation of these two cell types during tissue dissociation, as cells within the tightly-interacting NVU are challenging to fully dissociate, and cross-contamination is common. However, the analysis of previous datasets, as well as analyses of individual BICCN datasets showed greater separation of neurovascular cells and clearly demonstrated that CD19 expression is primarily in mural cells and not endothelial cells (FIGS. 1F, 9B, 13A).

### Example 8: The CD19 Isoform Recognized by CAR-T Cells is Expressed in the Adult Human Brain

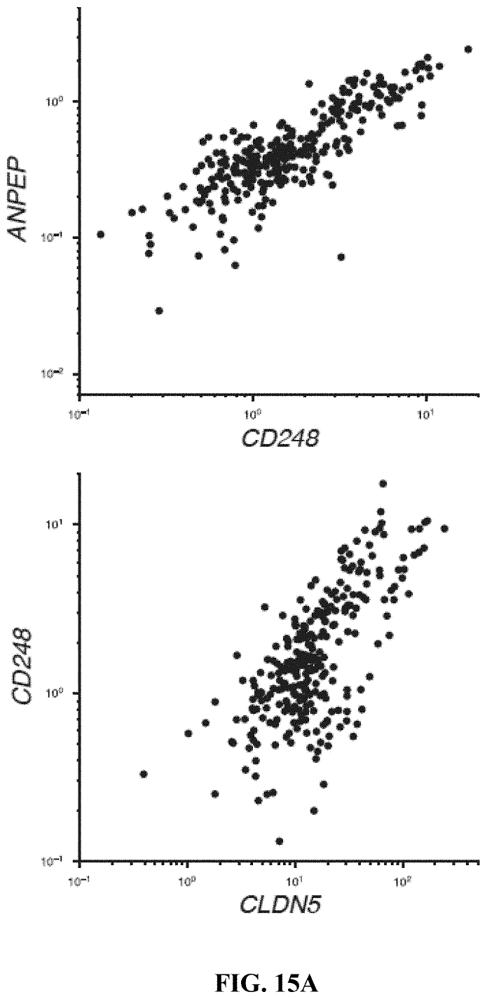

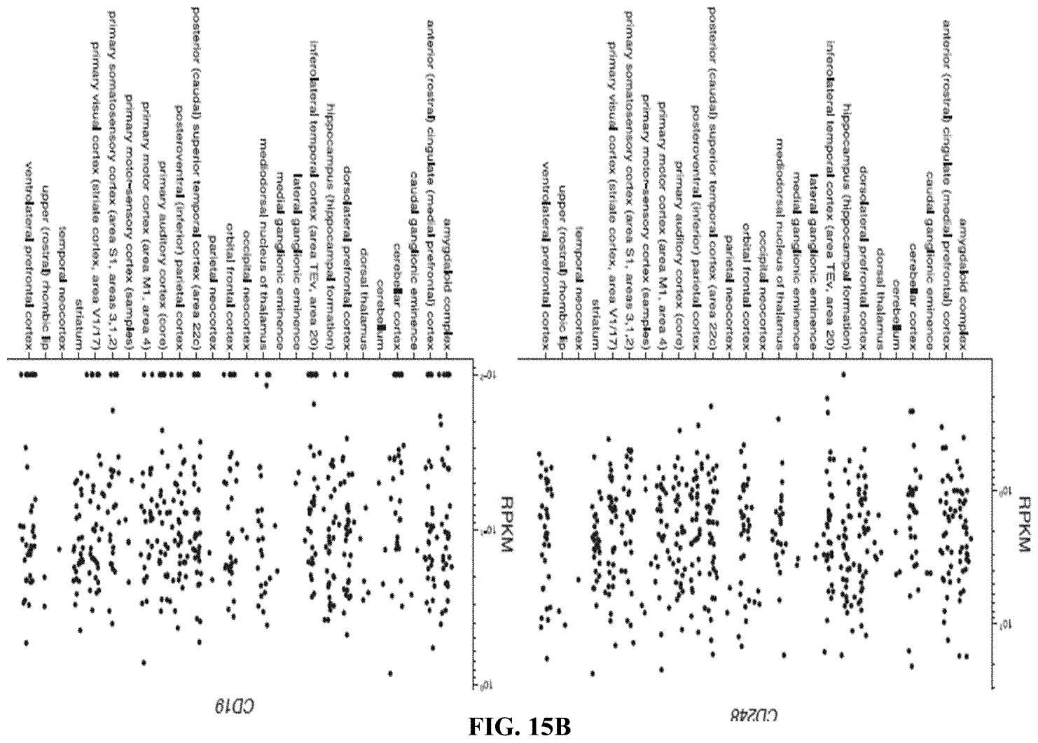

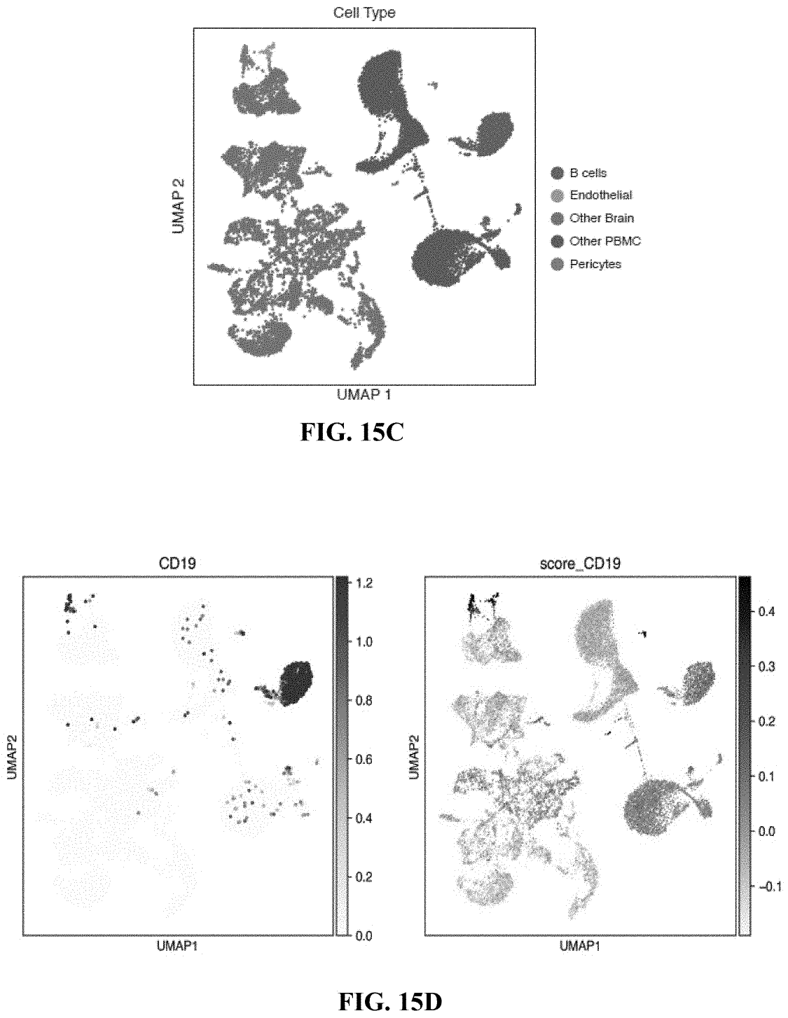

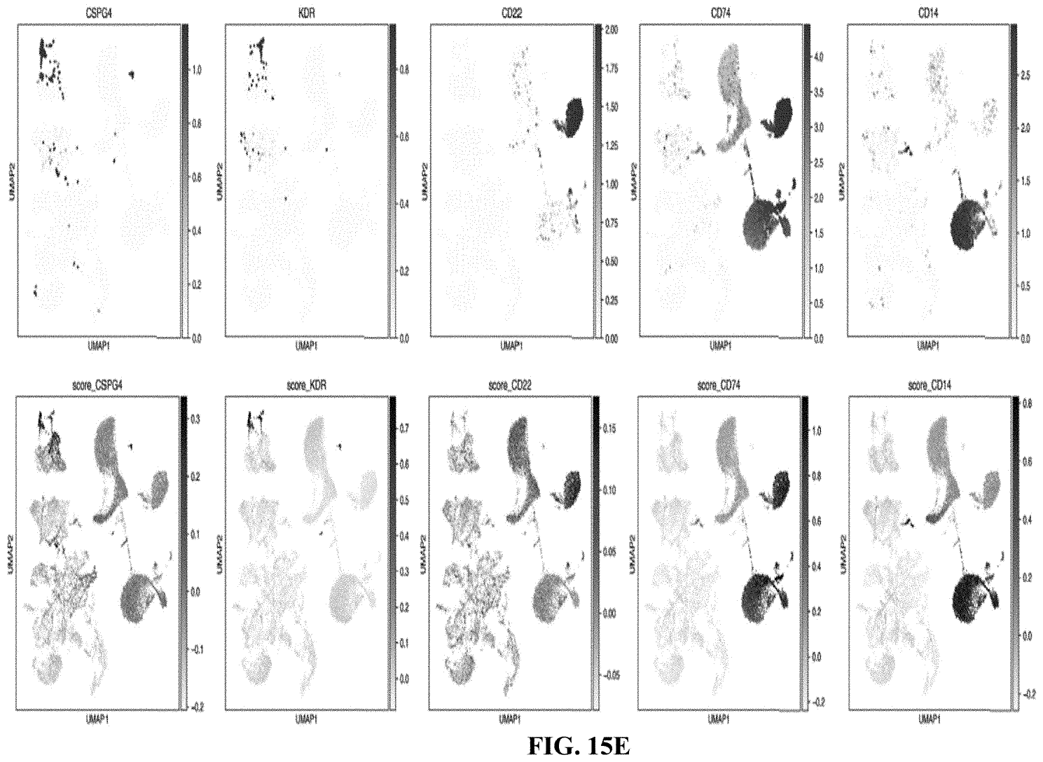

The expression of CD19 in the adult human brain was analyzed next to complement the results observed previously by immunohistochemistry. scRNA-seq analysis of mural cells in adult human brain samples is challenging, due to difficulties in tissue acquisition and low numbers of neurovascular cells in the brain, relative to neuronal or glial populations (FIG. 14A). Additionally, the majority of existing datasets are prospectively enriched for neuronal populations of interest. Therefore, to ask whether CD19 expression in pericytes is also present in adult samples, bulk RNA-sequencing data across human age and brain region generated by the Allen Institute Brainspan Project (Miller et al., 2014, Nature 508, 199-206) was utilized. This data contains more than 500 prenatal and postnatal samples from diverse brain regions (n=237 prenatal; n=287 postnatal). Since bulk tissue analyzed in different samples will have varying proportions of mural/vascular cells, the relative expression of mural genes across samples is a proxy for the underlying proportion of mural cells in the bulk tissue. For example, CD248 and ANPEP, two pericyte markers, are highly correlated in this data (FIG. 15A).

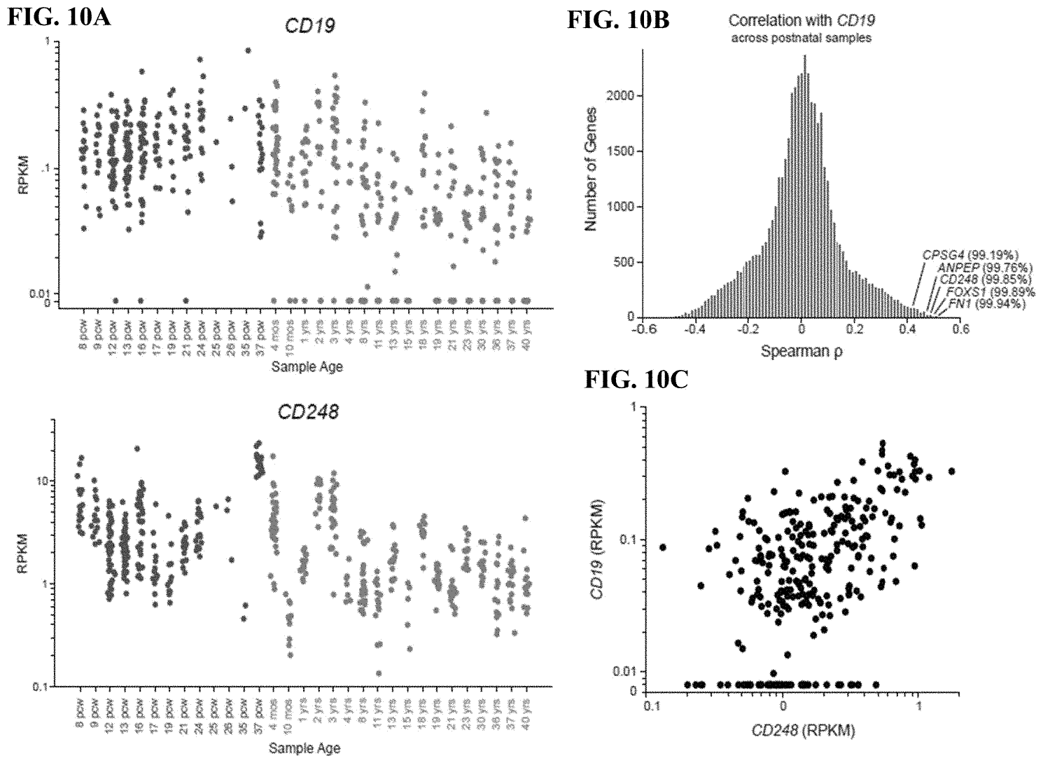

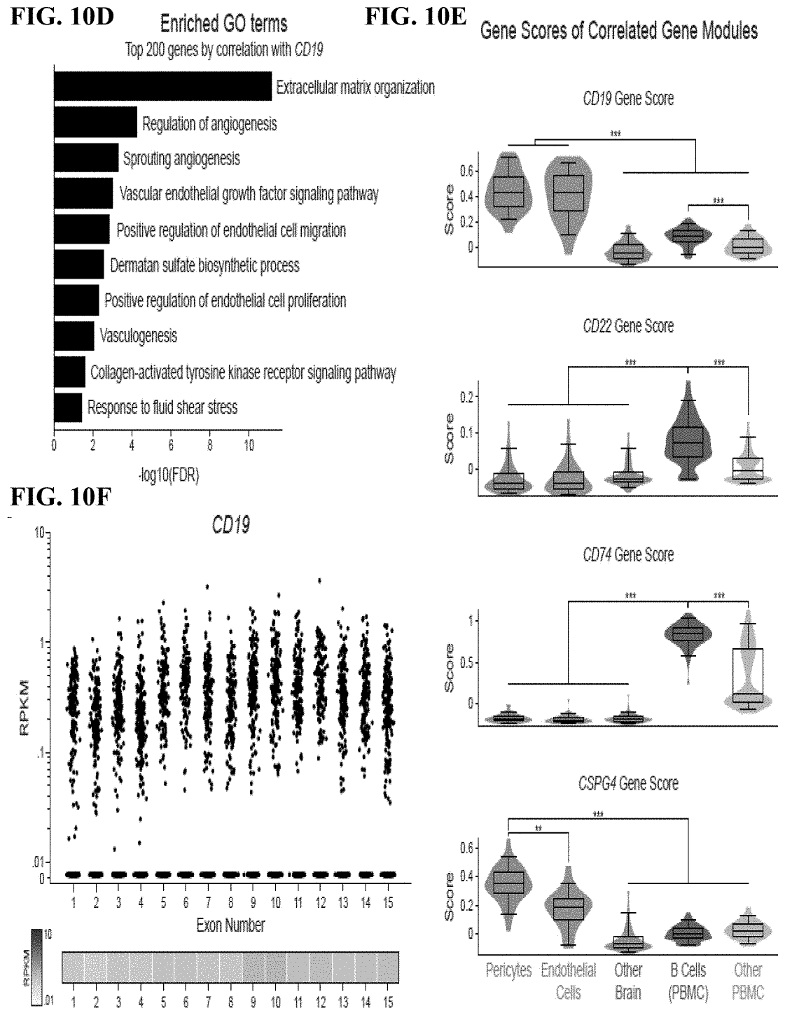

It was first confirmed that CD19 is expressed in both prenatal and postnatal samples at similar levels, and also expressed in samples from different brain regions (FIG. 10A, FIG. 15B). A genome-wide correlation analysis was performed across only the postnatal samples to identify genes that are co-expressed with CD19. Strikingly, this analysis showed that CD248, CSPG4, ANPEP, FOXS1, and FN1 were in the top 1% of genes correlated with CD19 in this data (FIGS. 10B-10C). Additionally, the top 200 genes most correlated with CD19 were enriched for GO terms associated with the NVU, such as angiogenesis, vasculogenesis, response to fluid shear stress, and multiple extracellular matrix related terms (FIG. 10D). This suggests that the expression of CD19 in the adult brain is primarily the result of mural cell abundance, rather than B cell abundance. The CD79-correlated gene module in adult brain was integrated with scRNA-seq data from the human brain and PBMCs. This allowed for the comparison of the gene module score (i.e., the enrichment of genes correlated with CD19 or another target) in brain pericytes, endothelial cells, B cells, in addition to other brain cells and PBMCs (FIG. 15C). As a control, the gene modules associated with CD22 and CD74, two highly expressed B cell-enriched genes, were enriched in B cells, and the CSPG4 gene module was highly enriched in pericytes (FIGS. 10E, 15D-15E). In contrast, the expression of the CD79-correlated gene module is highest in pericytes (FIG. 10E). CD19 expression seemed to decrease with age (FIG. 10A). This could be explained by two potential mechanisms: (1) either the proportion of CD19+ cells changes over time, or (2) the level of CD19 expression in mural cells changes over time. However, the concomitant decrease in CD248 expression with age, as well as prior studies support the former mechanism.

Lastly, it was asked whether the CD19 isoform that is expressed in the adult brain contains the specific CD19 epitope that is recognized by clinical CAR-T cells and BiTEs. The FMC63 scFv in clinical use recognizes an epitope encoded by exon 4 of CD19 (Sommermeyer et al., 2017, Leukemia 31, 2191-2199). Additionally, variants skipping exon 2 may also result in a lack of CD19 trafficking to the cell surface, also allowing evasion of FMC63 detection. An analysis of the mean expression of each CD79 exon in the adult human brain showed an even distribution of expression across exons, which clear expression of the key exons 2 and 4 (FIG. 10F).

### Example 9: Analysis of Neurotoxicity in Mouse Models of CD19 CAR-T Cell Therapy

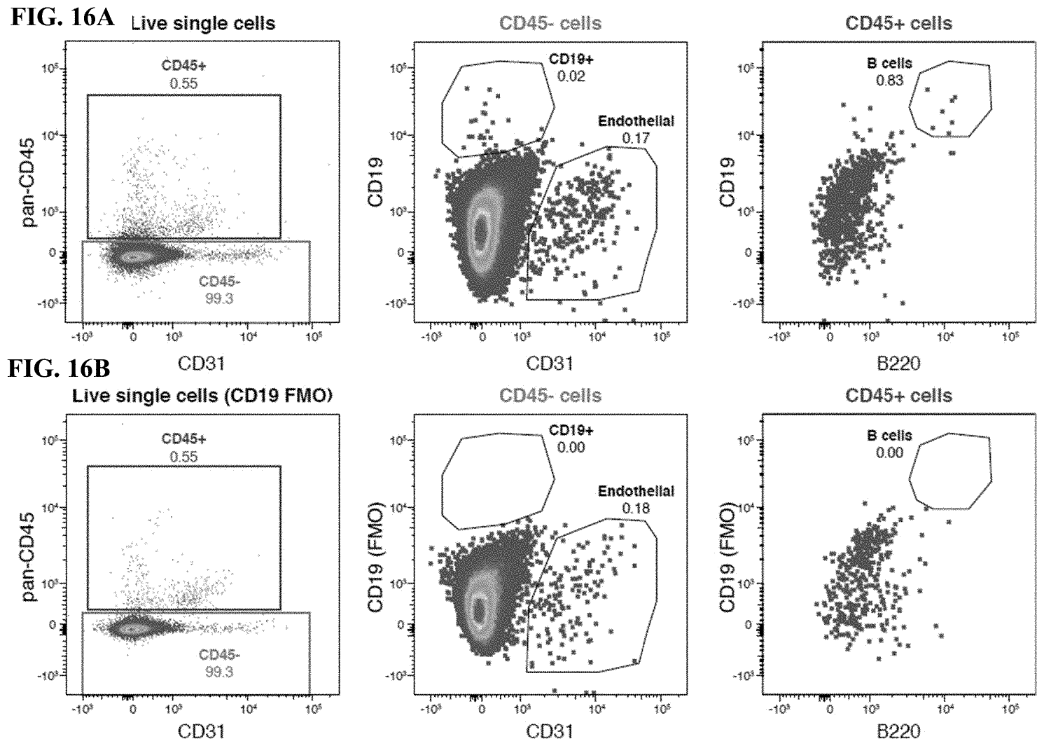

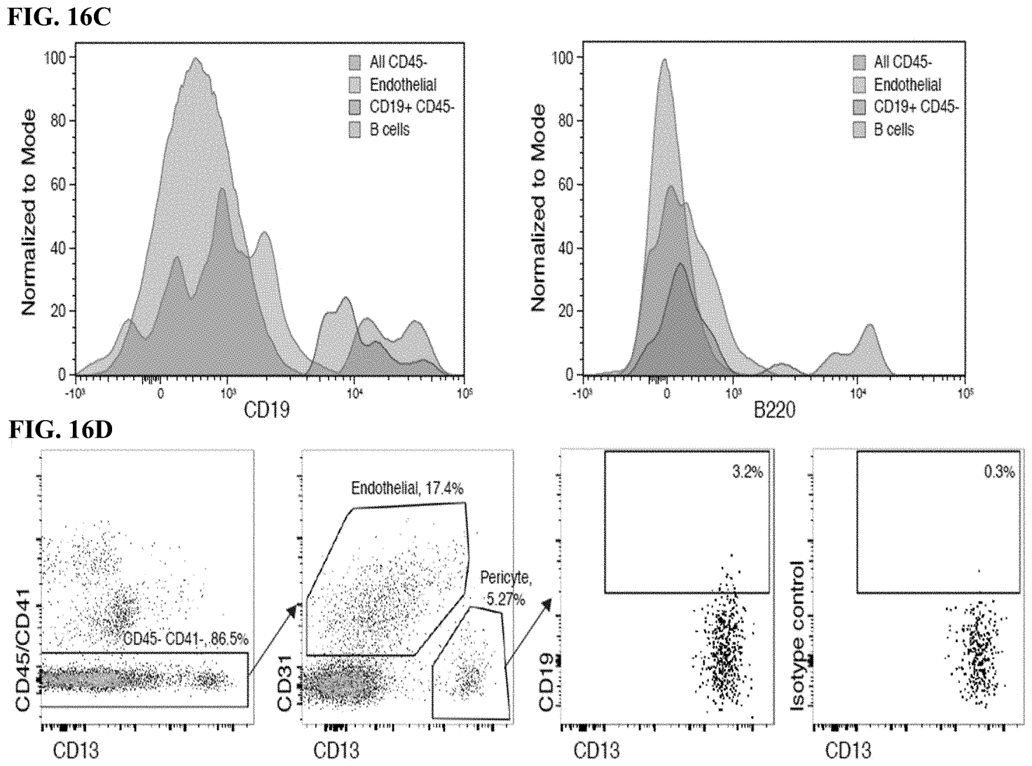

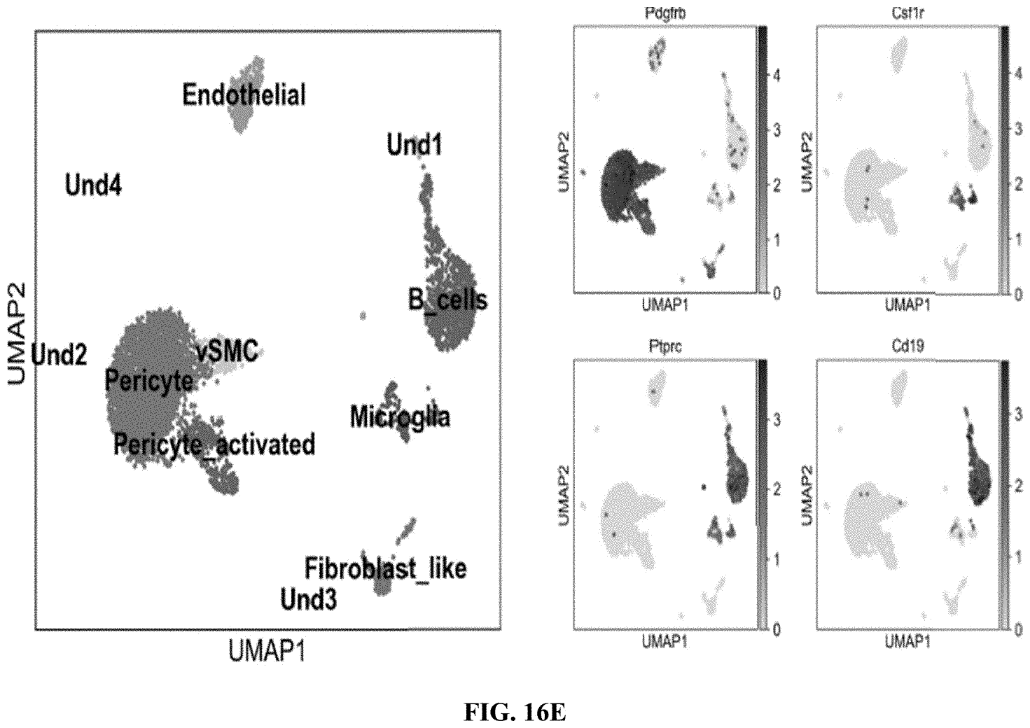

It was next asked whether the expression of CD19 in human mural cells is conserved in mice, which would allow for the use of mouse models to study mechanisms of human neurotoxicity. First, whole brains from healthy C57BI/6J mice were extracted and dissociated following a previously described protocol and analyzed the presence of CD19 by flow cytometry (Boroujerdi et al., 2014, Methods Mol. Biol. Clifton N.J. 1135, 383-392). This revealed the presence of a CD45-high CD19+ B cell population, as expected, and also revealed a rare population of CD45− CD19+ cells, which expressed CD19 at a similar level to B cells but did not express the B cell marker B220 (FIGS. 16A-16C). Pericytes, identified as a CD13+CD45-population, were indeed positive for CD19, albeit rarely, in contrast with what was observed in human data (FIG. 16D). To examine this at the transcriptional level, scRNA-seq was performed on cells isolated from dissociated whole mouse brain, followed by enrichment for CD19+, CD13+, and CD31+ cells. This analysis again confirmed low CD19 expression in pericytes relative to CD19+ B cells, and in fewer cells compared to scRNA-seq of human mural cells (FIG. 16E). Together, this data suggests that CD19+ mural cells are relatively less abundant in the mouse brain, compared to human brain.

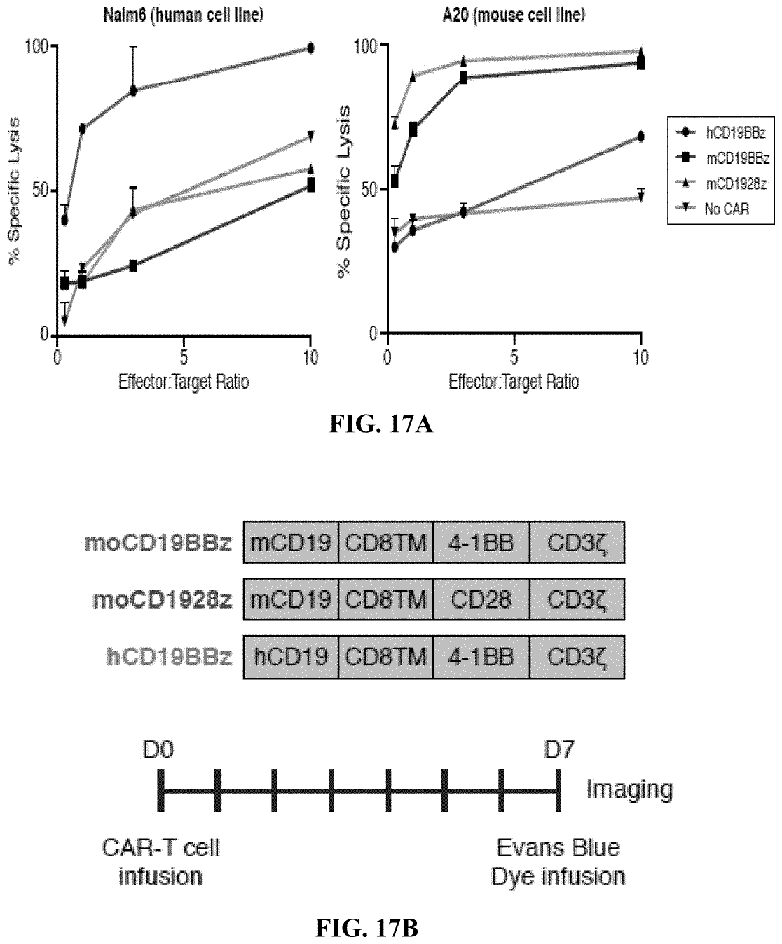

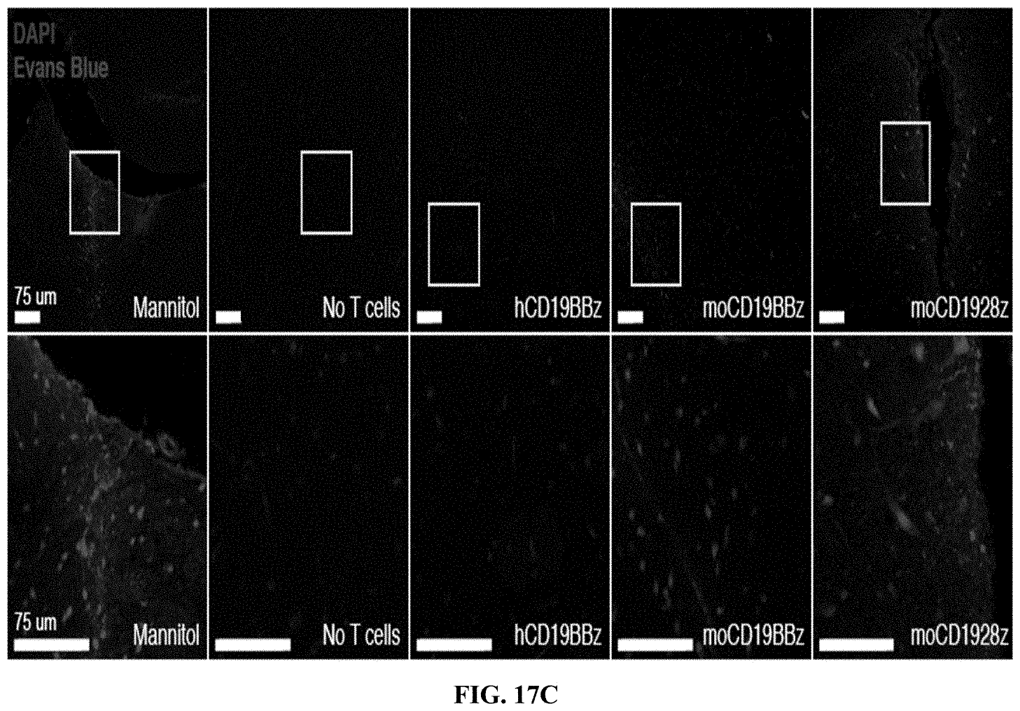

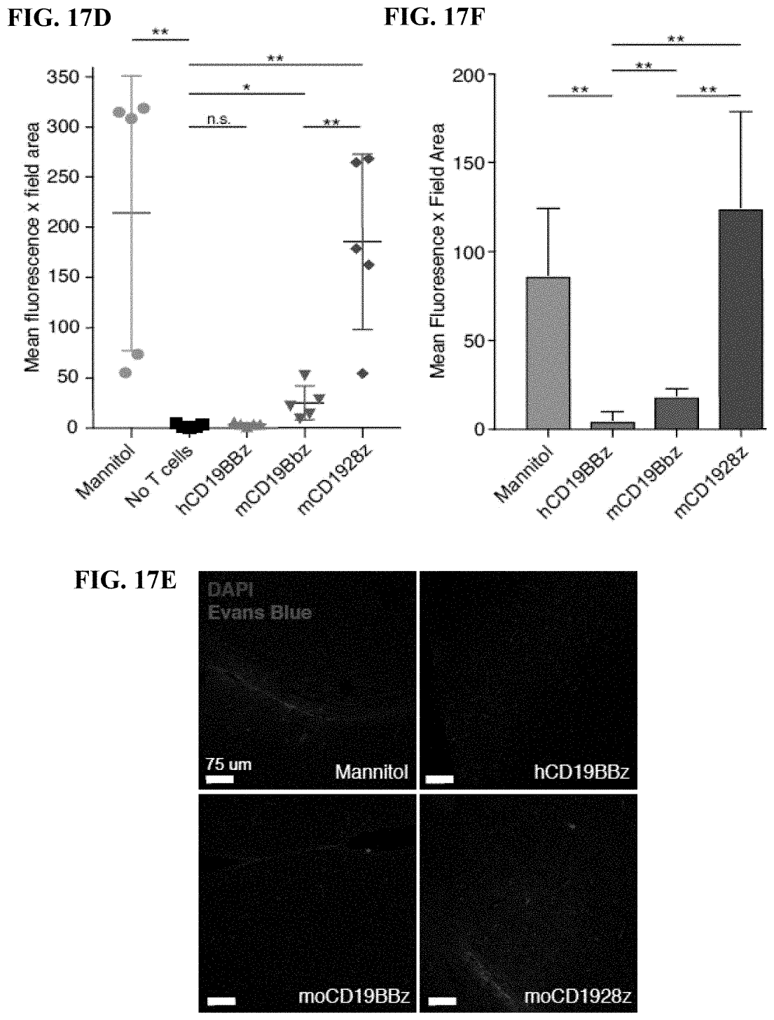

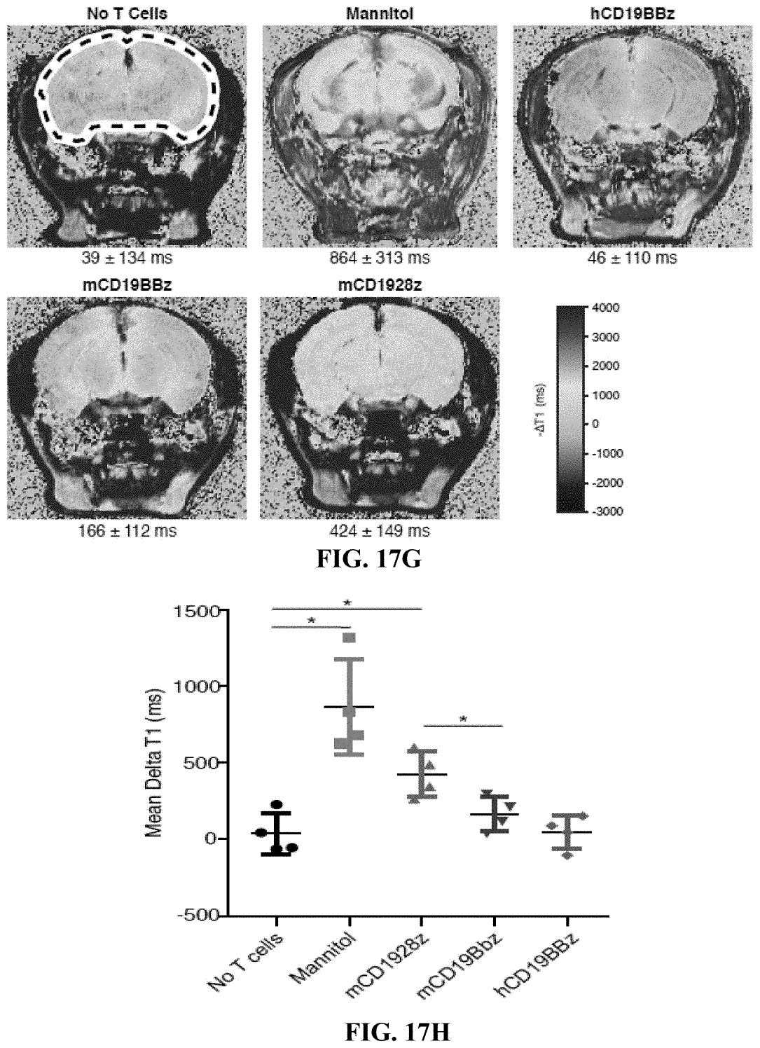

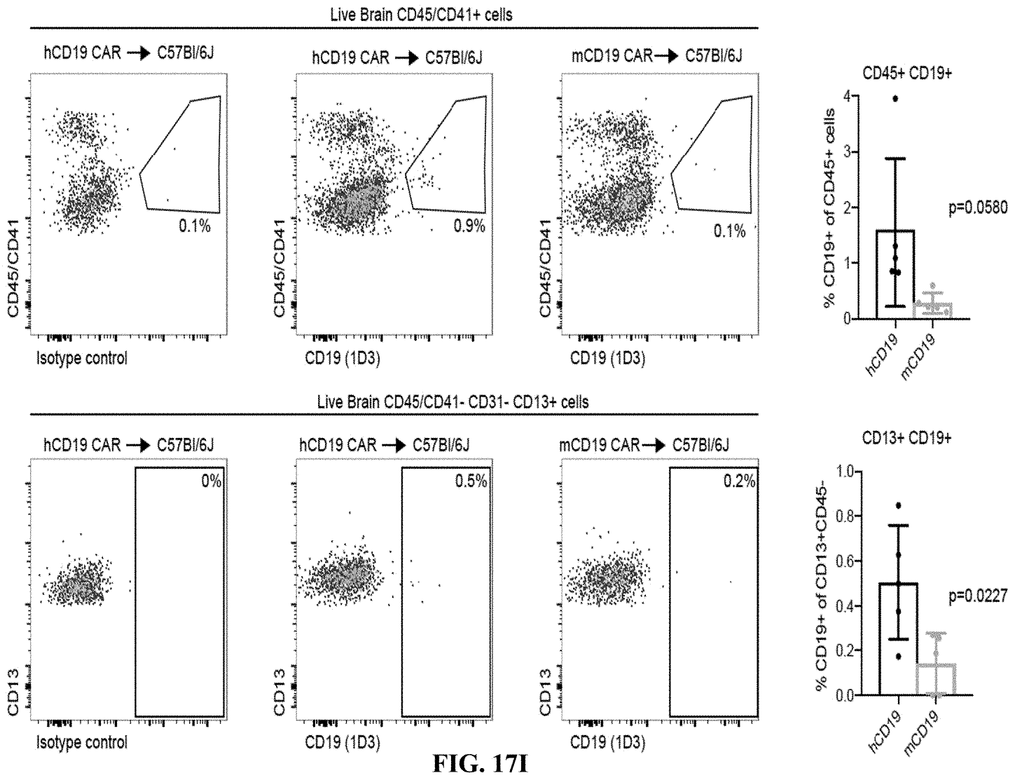

Given the presence of non-B CD19+ cells in mice, it was asked whether an infusion of CD19− directed CAR-T cells into immunodeficient, non-tumor bearing NOD.Cg-Prkdc^{scid} Il2rg^{tmWj1}/SzJ (NSG) mice would result in an observable neurologic phenotype. Since NSG mice do not develop B cells, any phenotype observed in this model is B cell independent and cannot be attributed to global effects related to CRS following B cell targeting. CD28 (28)-based or 4-1BB (BB)-based CAR-T cells, which differ in the signaling domain but are both used clinically, were generated targeting either human (FMC63 scFv) or mouse CD19 (1D3 scFv, the same as used for flow cytometry analysis above). This comprised three conditions: hCD19BBz, rep resenting a negative control that should not target murine CD19, mCD19BBz, and mCD1928z. Species-specific activation was confirmed using human and murine CD19+ B-ALL cell lines (FIG. 17A).

Seven days after infusion of CAR-T cells, NSG mice that had received murine-targeting, but not human-targeting, CD19 CAR-T cells displayed increased BBB permeability as measured by Evans Blue dye (EBD; FIGS. 17B-17D). EBD binds to albumin, which normally remains in the bloodstream under physiologic conditions; when the BBB is disrupted, albumin is able to cross into the brain parenchyma. A fourth group of mice receiving no transfusion displayed no EBD staining, and a fifth group that received mannitol at the time of EBD infusion, representing a positive control, displayed high EBD staining, as expected (FIGS. 17C-17D). The effect was higher in the mCD1928z compared to mCD19BBz CAR-T cell group, which is likely the result of stronger antigen receptor signaling provided by the CD28 domain. Indeed, prior studies have shown that the choice of costimulatory domain (CD28, 4-1BB, or others) strongly impacts sensitivity to antigen density with CD28-based CARs particularly sensitive to low levels of antigen density (Majzner et al., 2020, Cancer Discov. 10, 702-723). This experiment was repeated using a syngeneic, immunocompetent C57BI/7J model with pretreatment with cyclophosphamide as a lymphodepleting preconditioning regimen. The syngeneic study recapitulated the pattern of BBB permeability observed in the NSG model, suggesting that the presence of murine CD19+ B cells in the syngeneic model did not affect the specific disruption of the BBB observed only in the murine-targeting conditions (FIGS. 17E-17F). BBB integrity was also measured four days after CAR-T cell transfusion in NSG mice by measuring gadodiamide uptake using 9.4 tesla magnetic resonance imaging. This showed an increase in uptake in the mice that had received murine-targeting, but not human targeting, CD19 CAR-T cells (FIGS. 17G-17H). Finally, brains from treated mice were dissociated and flow cytometry performed to measure CD19+ pericyte depletion following CAR infusion. Mice receiving mCD1928z, but not hCD1928z, showed a decrease in both CD45+CD19+ B cells as well as CD45-CD13+CD19+ mural cells (FIG. 17I). Behavioral changes were not specifically analyzed, but no overt behavioral phenotype was observed.

In summary, these experiments suggest that despite a lower frequency of CD19 expression in mouse mural cells, compared to human mural cells, administration of CD19-directed CAR-T cells can cause BBB leakiness and pericyte depletion in mice lacking B cells. It is possible that some of this effect may be mediated by targeting of microglia, which were previously demonstrated to be depleted following CD19-directed CAR-T cell treatment in mice, and also display low expression of CdJ9 in our scRNA-seq data, as well as the Tabula Muris database (Tabula Muris Consortium et al., 2018, Nature 562, 367-372). Therefore, these experiments demonstrate that while some aspects of CAR-T function and toxicity may be measured in preclinical mouse models, there are important limitations, since species-specific differences in cell type and transcriptional state may not perfectly match human-specific pathophysiology. Indeed, initial CAR-T cells studies in mouse models did not predict the degree of neurotoxicity that was later observed in human clinical trials (Ruella and June, 2018, Mol. Ther. J Am. Soc. Gene Ther. 26, 1401-1403).

### Example 10: Human Brain Mural-Specific Expression of CD19

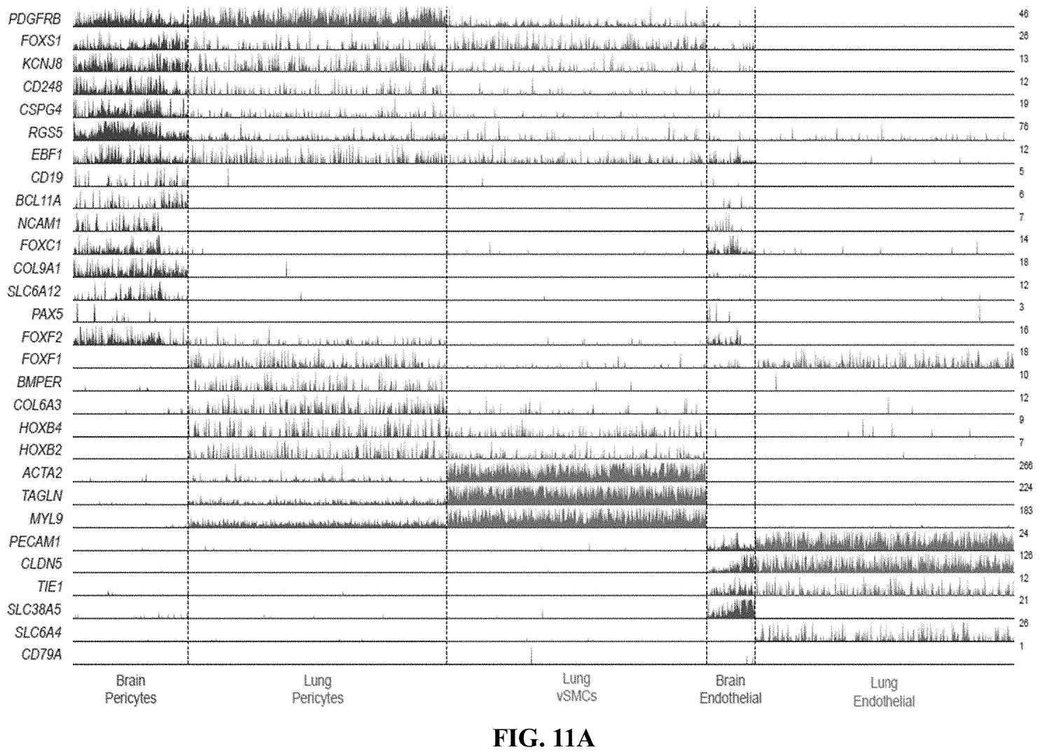

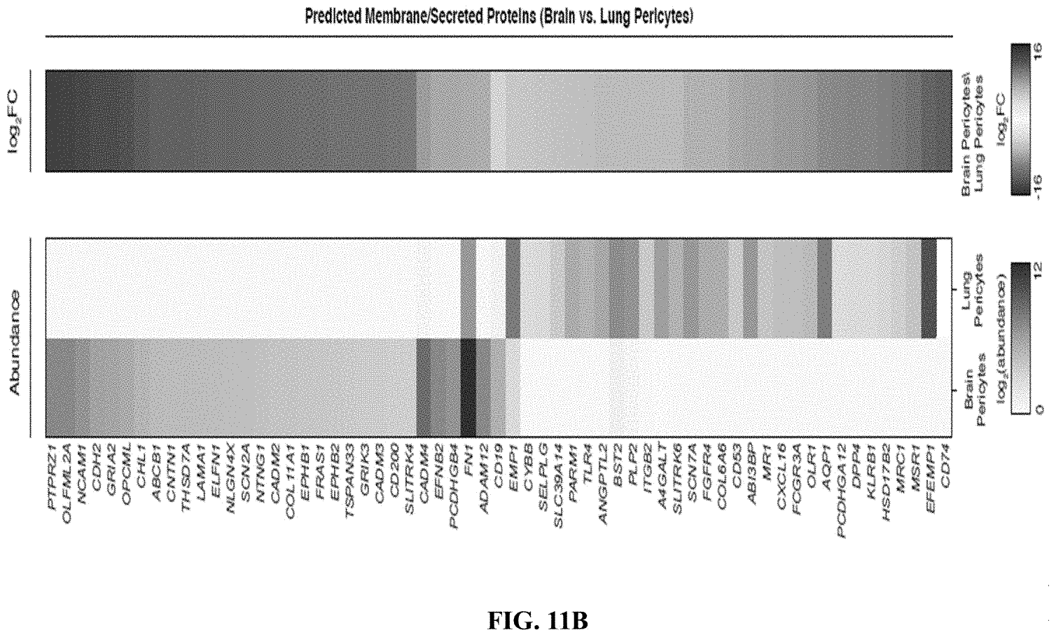

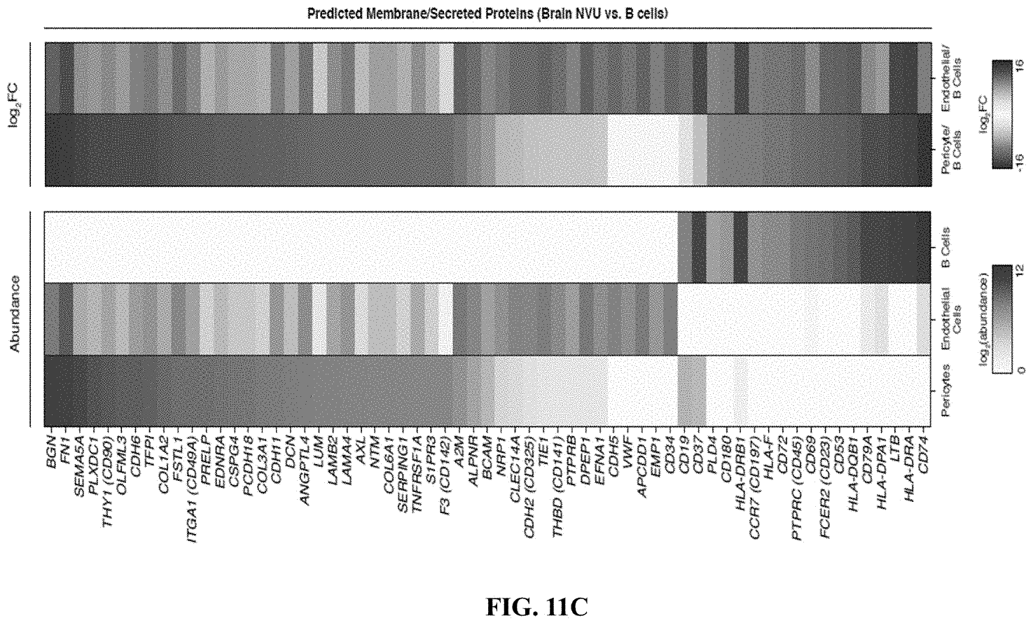

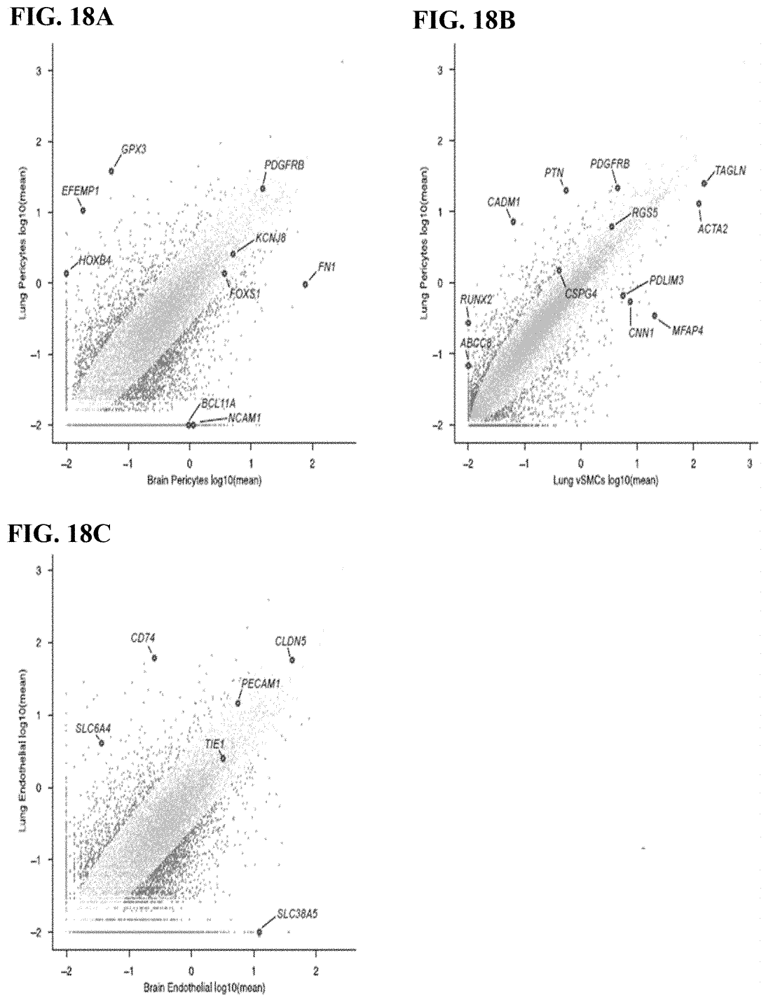

Since pericytes are present in multiple organs, a comparative analysis was performed of brain pericytes with pericytes and vSMCs from the lung, a highly vascularized tissue with high numbers of pericytes and endothelial cells. Although all mural cell populations showed shared expression of a core transcriptional identity, such as PDGFRB, RGS5, FOXS1, and KCN8, numerous transcriptional differences were identified between brain and lung pericytes (FIGS. 11A, 18A). Notably, CD19 is specifically expressed in brain, but not lung mural cells. Only 6/2724 lung mural cells had any detectable counts for CD19, showing no apparent enrichment over nonspecific counts found in all non-B cell clusters. This organ specificity may be explained by broader differences in lineage-specific transcription factors between the two cell types, which agrees with the distinct developmental origins of brain pericytes from neural crest cells. Namely, brain pericytes express BCL11A, and lowly express PAX5, two key developmental factors in brain development but also B cell development. Interestingly, EBF1, another transcription factor important for B cell development, was expressed by both populations. The expression of these B cell factors in brain mural cells may explain the specific expression of CD19 expression in these cells.

Finally, it was asked whether genes predicted to localize to the membrane or cell surface were differentially expressed in the NVU relative to lung pericytes as well as B cells and could this be used to improve the safety of CD19-directed CAR-T (FIGS. 11B-11C). For example, recent studies have shown the utility of “AND” logic gates to improve the specificity of CAR-T cells by requiring recognition of two distinct antigens. The most highly differentially expressed genes among those annotated were identified as being secreted or located on the cell surface, in brain versus lung pericytes, as well as B cells versus brain pericytes and endothelial cells. These could improve the specificity of B cell-directed CAR-T cells by requiring dual recognition of two antigens. This analysis revealed additional genes, such as CD74, HLA-DRA, and LTP, which are both highly expressed and highly enriched on B cells relative to brain pericytes (FIG. 11C). An alternative approach to improving CAR-T cell specificity is to use “NOT” gates, where inhibitory CAR constructs recognize nonspecific target proteins and prevent T cell activation. Numerous genes that are enriched in pericytes over B cells, such as BGN, FN, and SEMAA were identified in the present study (FIG. 11C).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 11

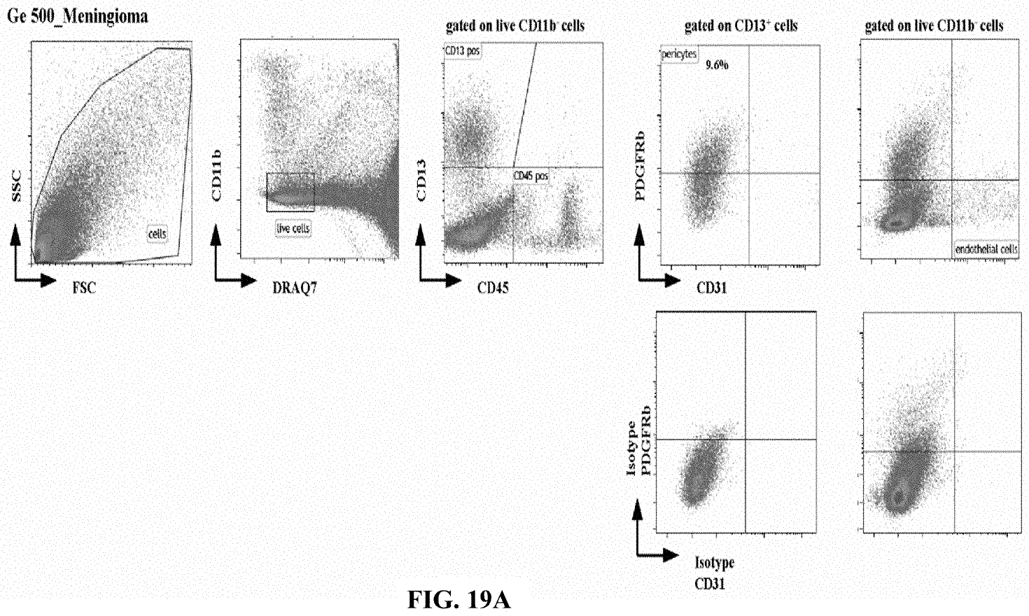

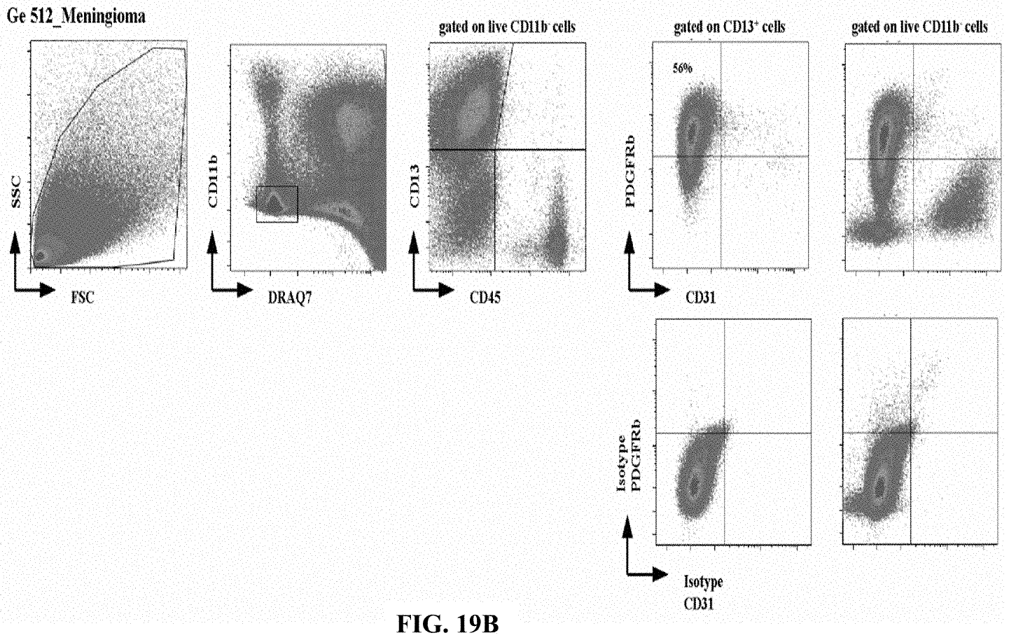

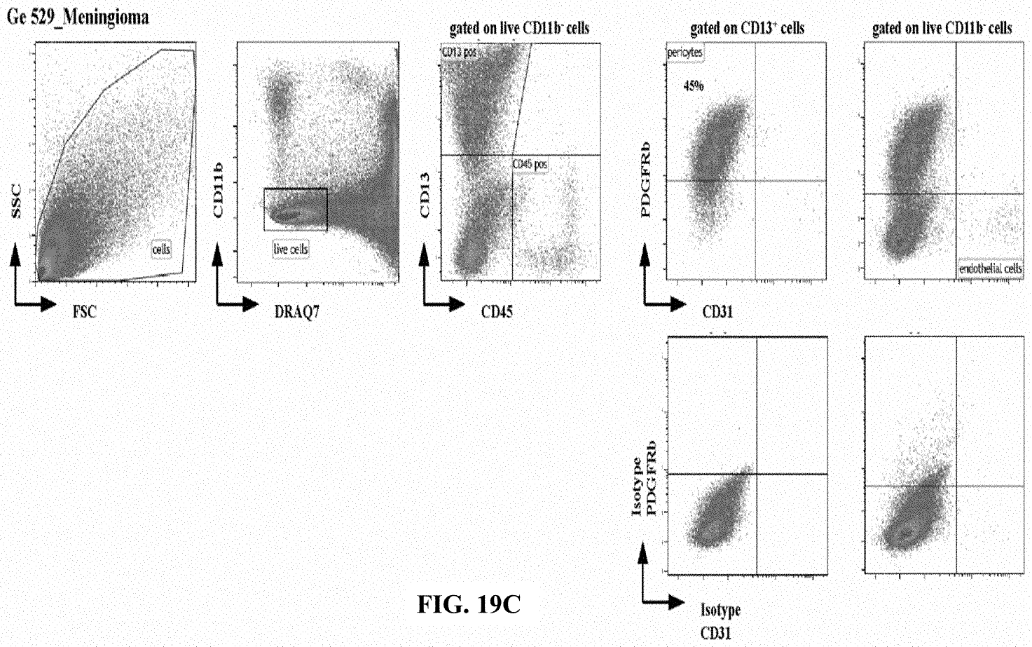

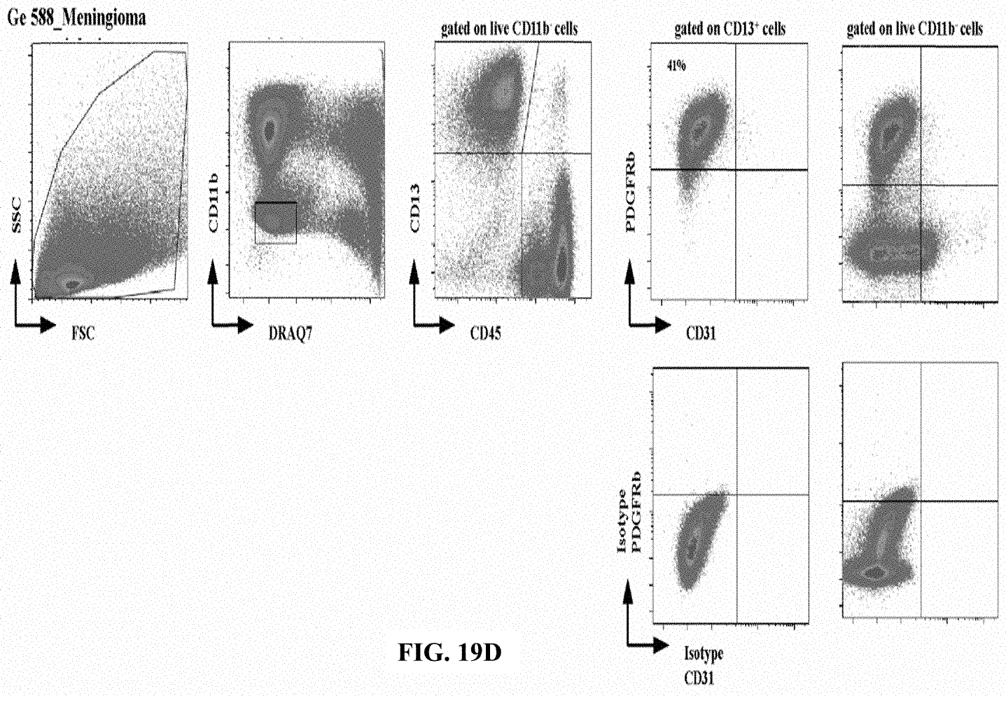

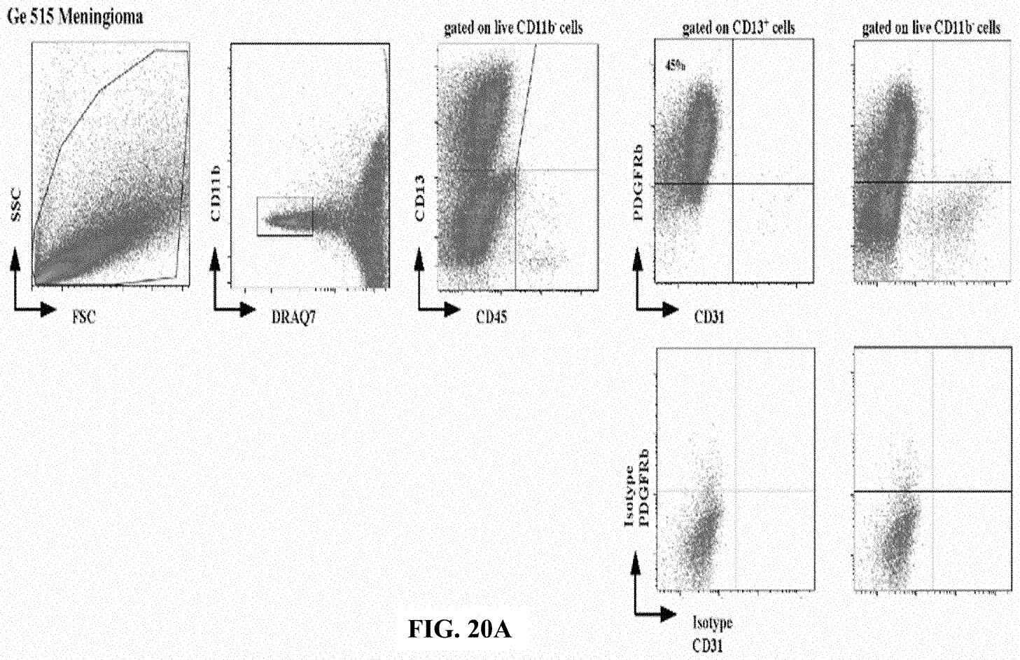

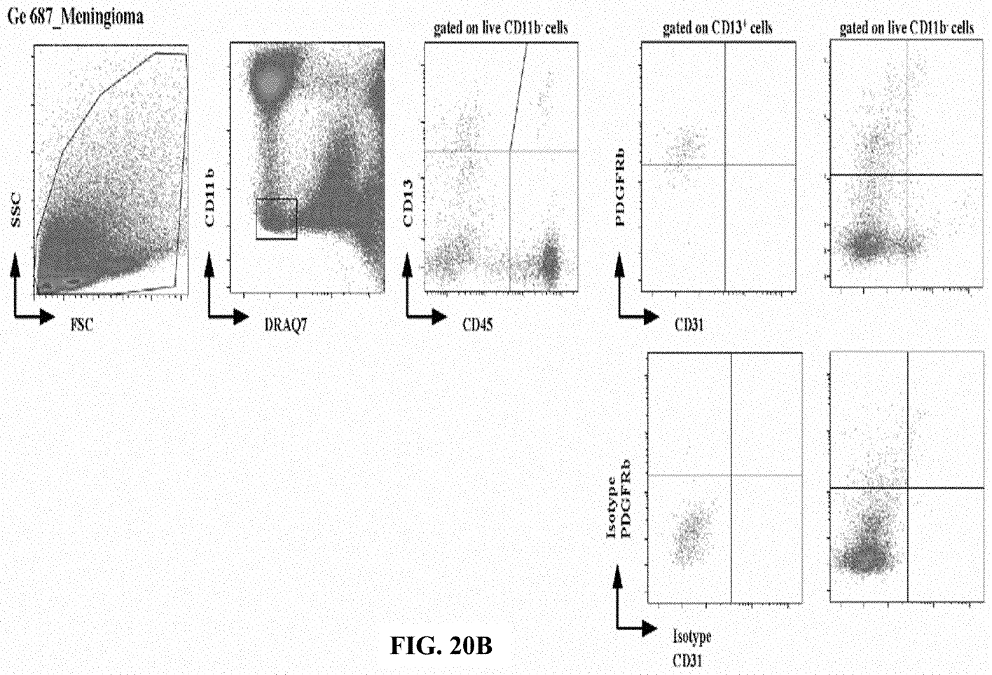

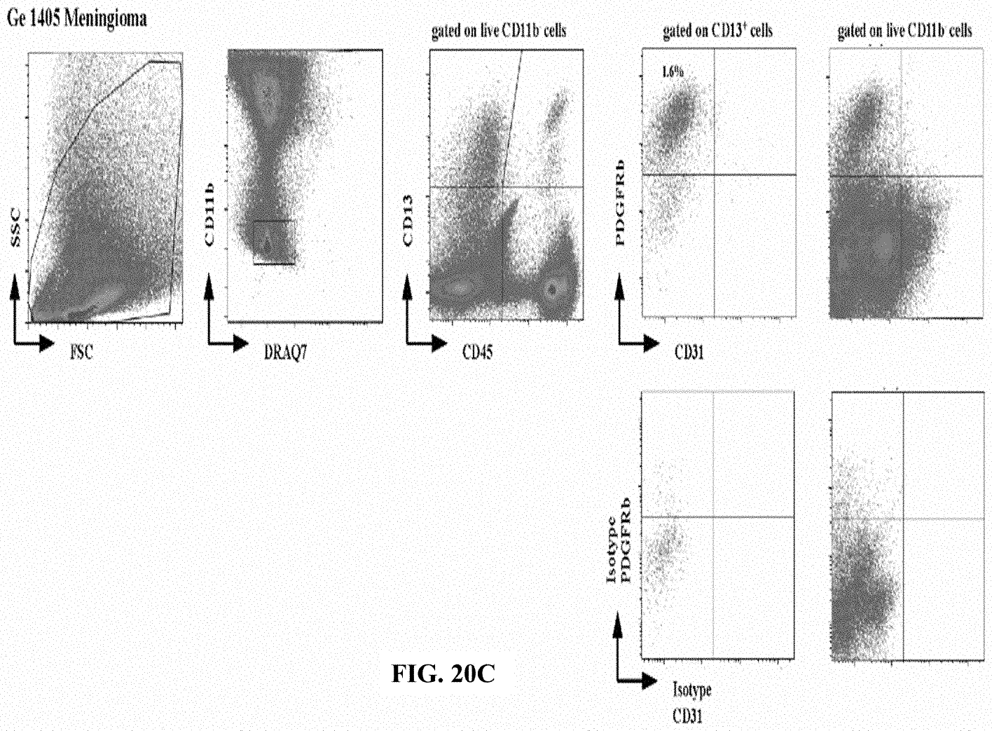

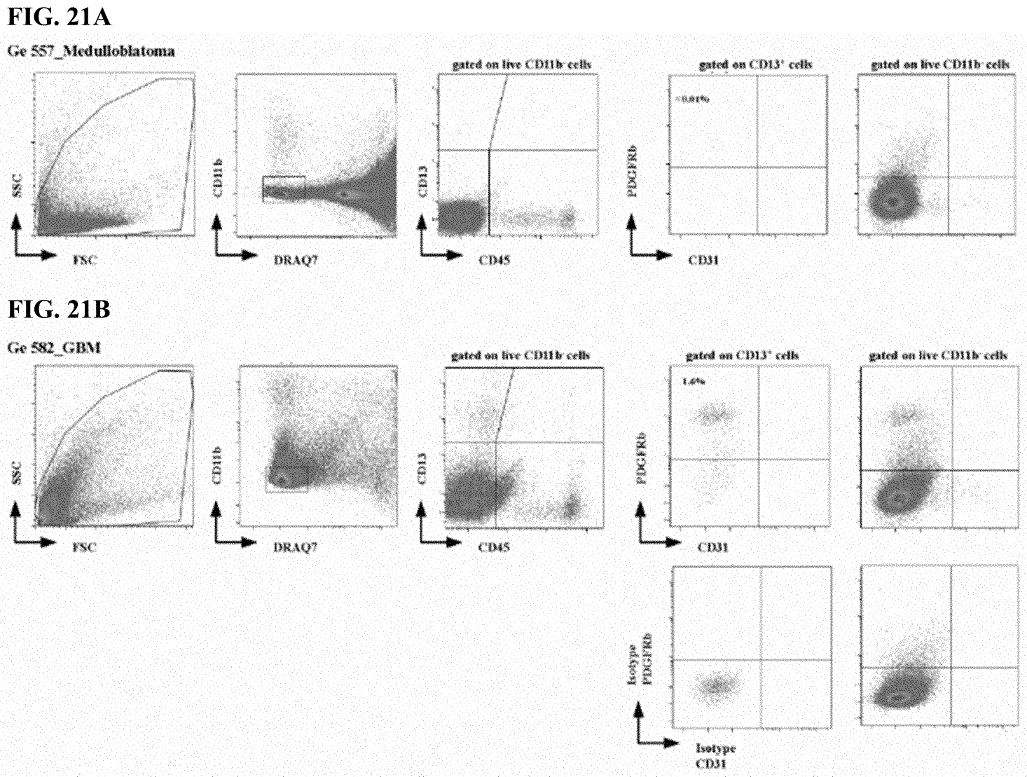

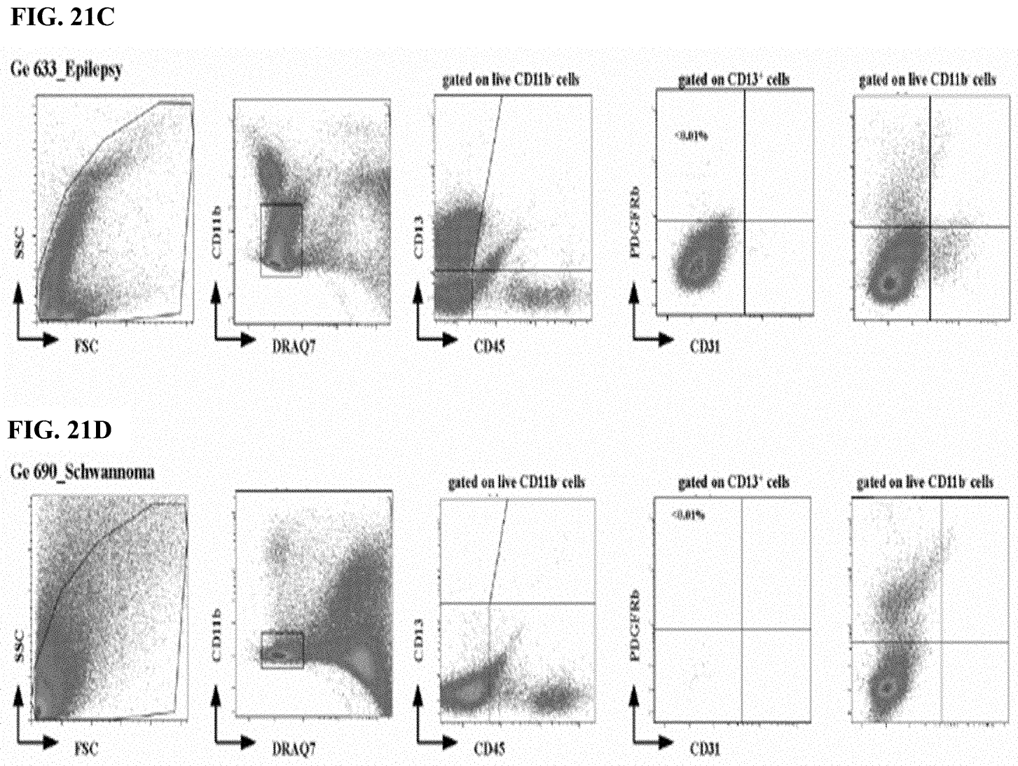

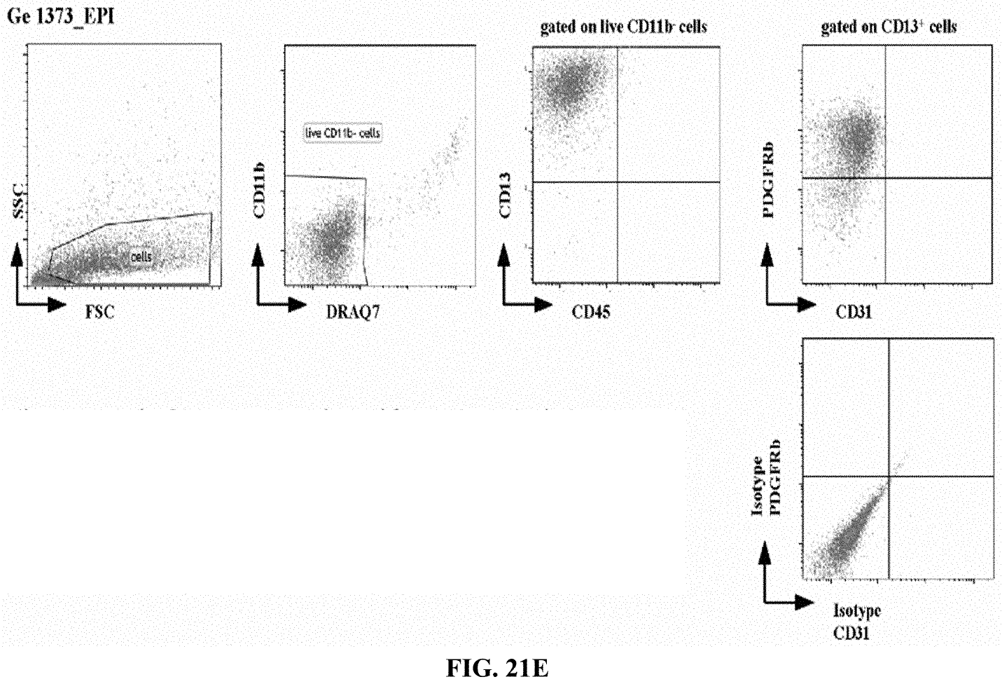

In order to further characterize the expression of CD19 in brain pericytes, analysis and isolation strategies were developed to identify these cells. Seven meningioma samples were digested enzymatically and stained with antibodies to CD11b, CD45, CD13, PDGFRb, CD31, and CD19 (FIGS. 19A-19D, 20A-20C). Isotypes to PDGFRb and CD31 were used. Draq7 was used to remove dead cells. Pericytes were identified as CD13+ PDGFRb+CD31− live CD11b-cells. For samples Ge 512, Ge 515, and Ge 1405, CD13+ PDGFRb+CD31− live CD11b− cells were sorted as pericytes, whereas CD31+ live CD11− cells were sorted as endothelial cells. The numbers in the quadrants depict the frequency of CD13+ PDGFRb+CD31− live CD11b-pericytes, which ranges from 9.6-56% in meningioma samples. RNA was extracted from sorted cells and used for RNA sequencing analysis to confirm pericyte/endothelial cell subset identification. Additional pericyte identifications were performed in samples from patients with Medulloblastoma (n=1) (FIG. 21A), GBM (n=1) (FIG. 21B), epilepsy (n=1) (FIG. 21C), and schwannoma (n=1) (FIG. 21D). These samples were digested enzymatically and stained as described in FIG. 20A-20C. Pericytes were identified as CD13+ PDGFRb+CD31− live CD11b-cells. The numbers in the quadrants depict the frequency of CD13+ PDGFRb+CD31− live CD11b− pericytes.

Pericytes were isolated from digested epilepsy sample Ge 1373 and cultured (FIG. 21E). After one month, cells were stained with antibodies to CD11b, CD45, CD13, PDGFRb, CD31 and CD19. Isotypes to PDGFRb and CD31 were used. Draq7 was used to remove dead cells. Pericytes were identified as CD13+ PDGFRb+CD31− live CD11b− cells, demonstrating the ability to selectively isolate this cell type from primary samples for further downstream applications, such as functionality testing of iCARs.

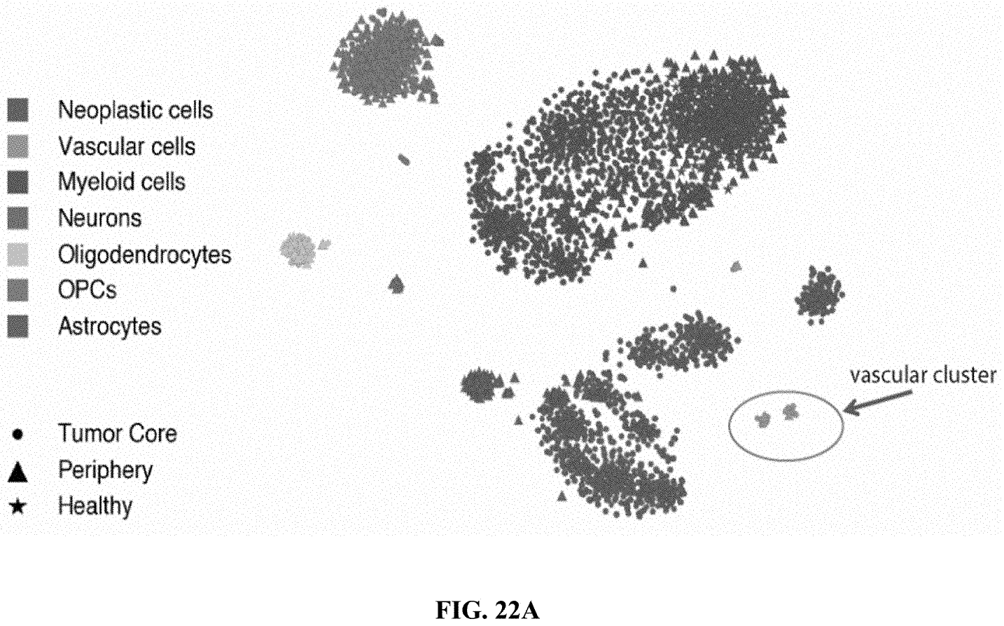

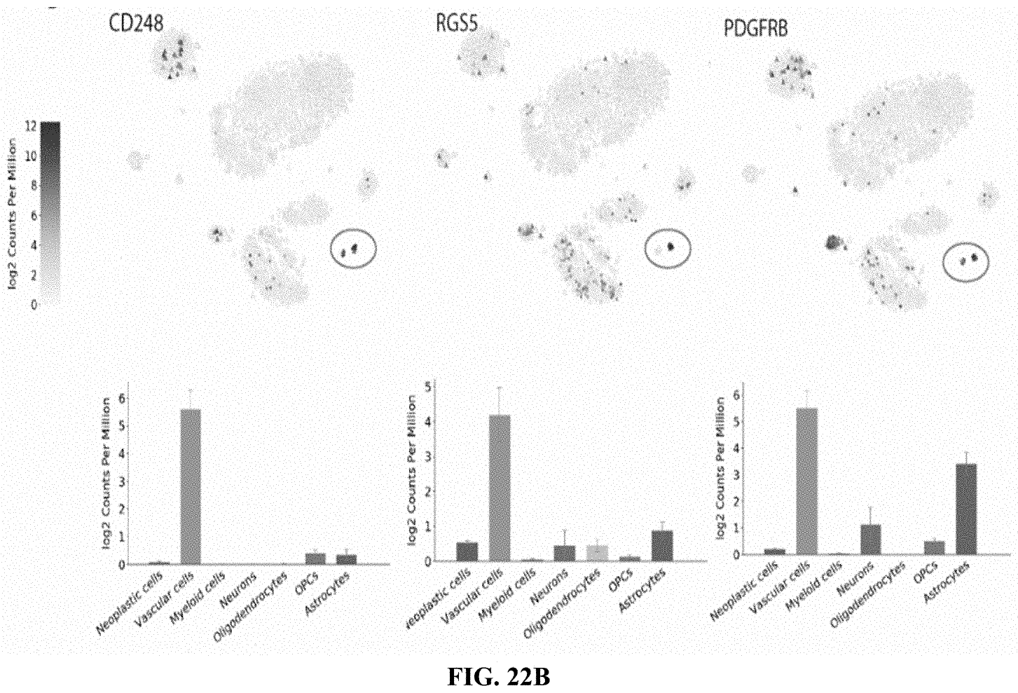

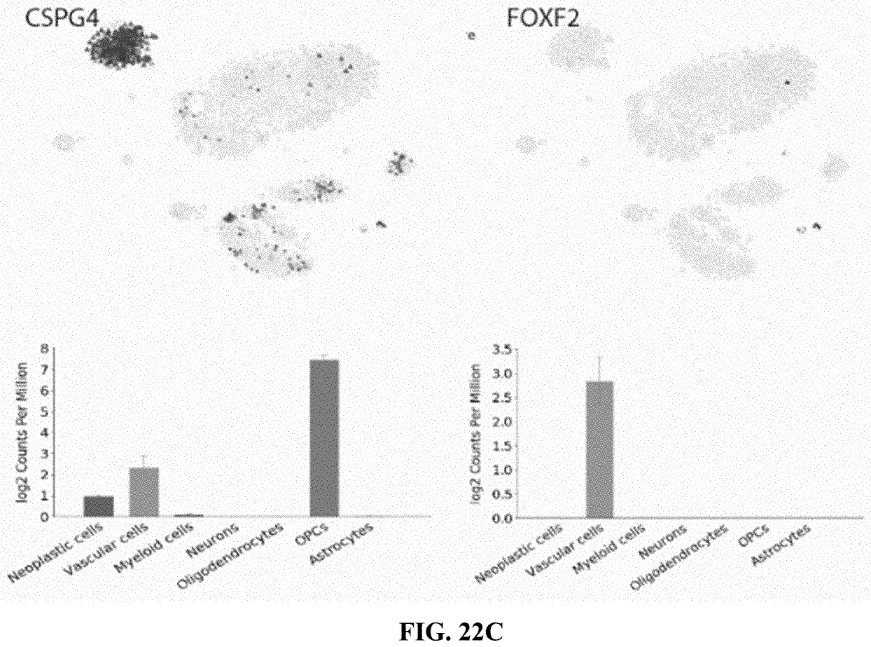

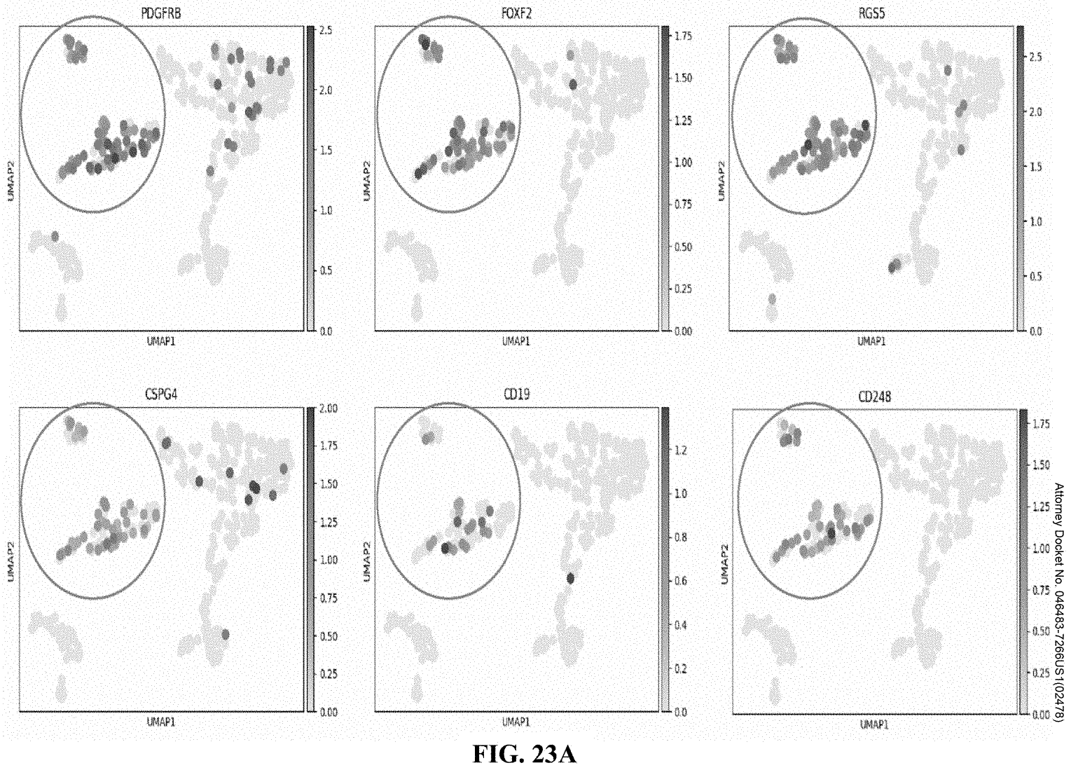

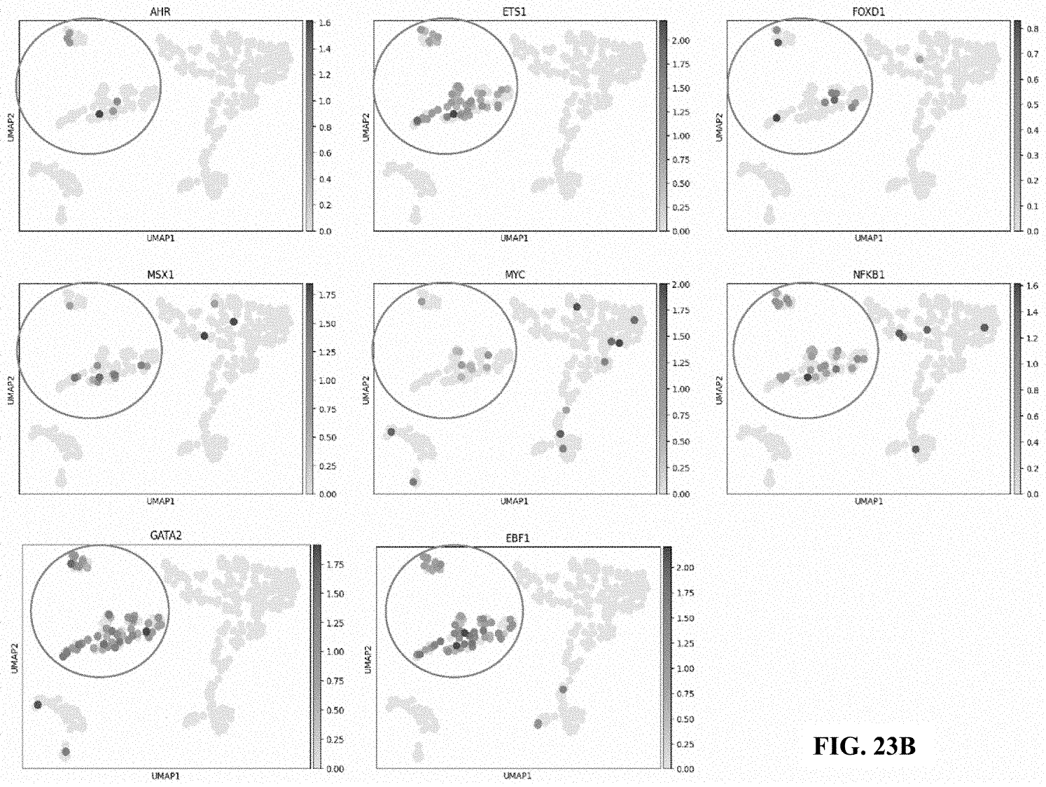

Pericytes can be identified transcriptionally through the transcriptional analysis of pericyte markers using single cell transcriptomic datasets (Darmanis et al., (2017) Cell reports, 21(5), 1399-1410). The Vascular cell cluster is enriched in pericyte markers (FIG. 22A-22C). In order to understand the transcriptional control that drives CD19 expression, overlaying of single-cell transcriptomics of pericyte markers (FIG. 23A) and transcription factors controlling CD19 expression (FIG. 23B) in pericytes analyzed through single cell transcriptomic datasets (Developmental timepoint and Brain region GW22T_thalamus from Human—Single Cell 10× RNAseq—Kriegstein) can provide indications of which transcription factors drive CD19 expression outside of the context of B cells.

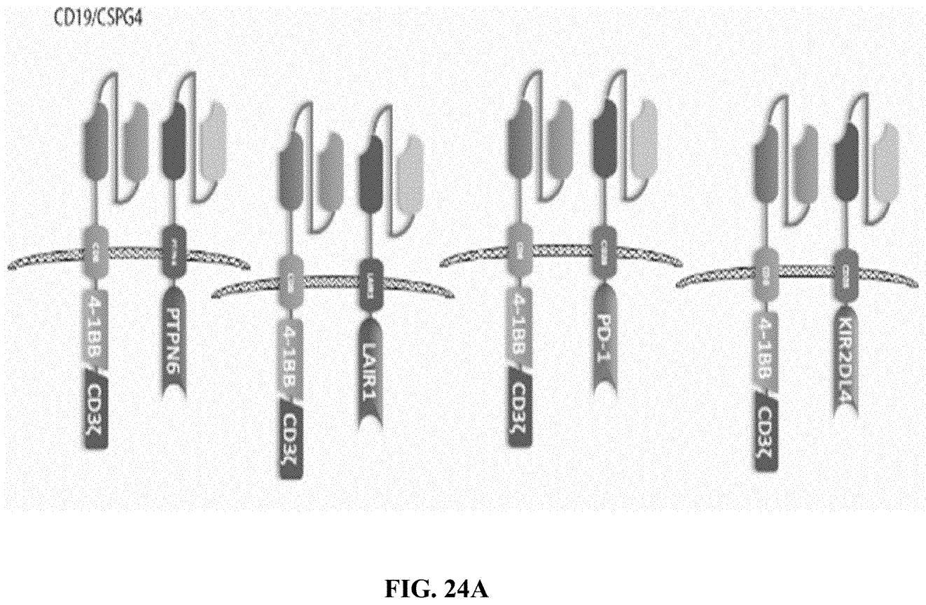

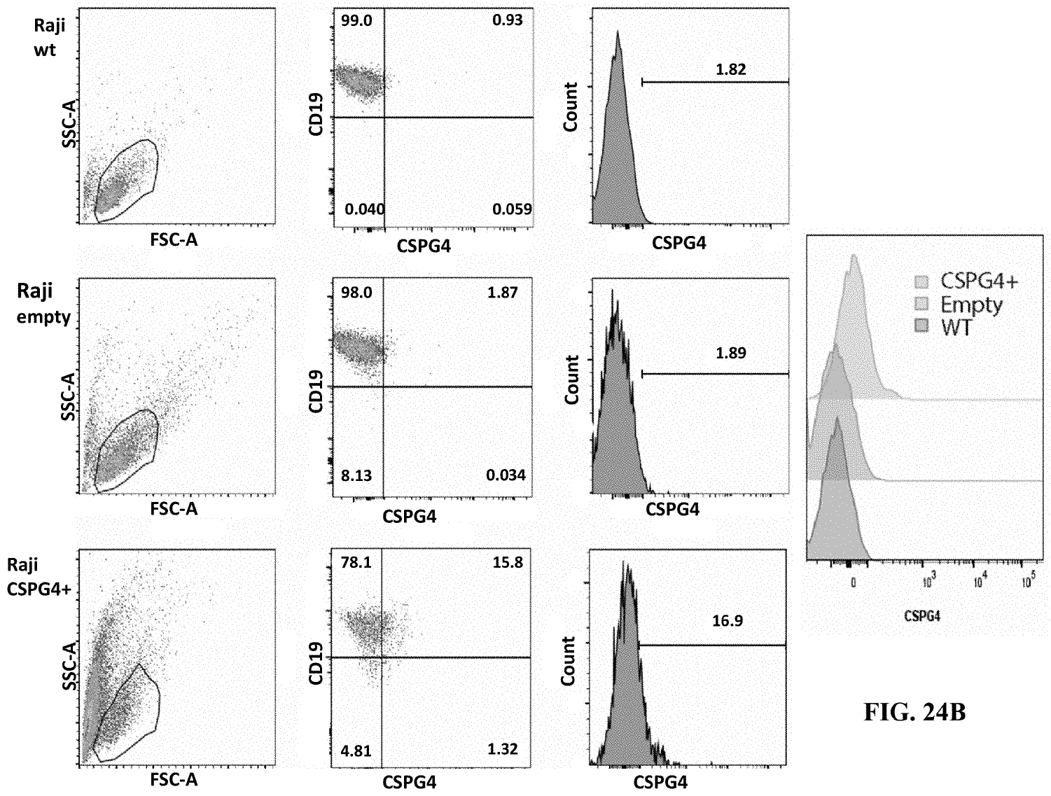

Inhibitory CARs to prevent CD19 CAR-related neurotoxicity are generated in two separate formats: bispecific (activatory anti-CD19 and inhibitory anti-pericyte CARs) or monospecific inhibitory CARs (FIG. 24A, FIG. 25). Inhibitory CARs can contain intracellular signaling domains from PTPN6, LAIR1, PD-1, and KIR2DL4. To evaluate the function of iCARs, a representative target cell line expressing CD19 and a pericyte-specific marker, such as CSPG4, is required. This target cell line can be generated by expressing CSPG4 in the CD19+ B cell lymphoma line Raji. Since the expression of CSPG4 is low in the Raji cell line, a novel expression vector is used where both GFP and CSPG4 expression are under the control of a single strong constitutive human promoter, EF1alpha (FIG. 24B).

What is claimed is:

1. A modified immune cell comprising a chimeric antigen receptor (CAR) and an inhibitory CAR (iCAR),
wherein the CAR comprises an antigen-binding domain capable of binding CD19, a transmembrane domain, and an intracellular domain, and
wherein the iCAR comprises an antigen-binding domain capable of binding a pericyte-associated or pericyte-specific antigen, and an inhibitory signaling domain.

2. The modified immune cell of claim 1, wherein the antigen-binding domain of the CAR and/or iCAR is an antibody or an antigen-binding fragment thereof.

3. The modified immune cell of claim 2, wherein the antigen-binding fragment is a Fab or a scFv.

4. The modified immune cell of claim 1, wherein the antigen-binding domain of the iCAR is an extracellular domain of a pericyte-associated or pericyte-specific ligand.

5. The modified immune cell of claim 1, wherein the antigen-binding domain of the iCAR binds to an antigen expressed on the surface of a pericyte, wherein the antigen is not CD19.

6. The modified immune cell of claim 1, wherein the antigen-binding domain of the iCAR binds to an antigen selected from the group consisting of CD146, PDGFRB, RGS5, CSPG4, CD248, BGN, FN1, SEMASA, PLXDC1, THY1, CDH6, TFPI, COL1A2, ITGA1, EDNRA, PCDH18, CDH11, AXL, NTM, TNFRSF1A, S1PR3, and F3.

7. The modified immune cell of claim 1, wherein the inhibitory signaling domain of the iCAR comprises an inhibitory signaling domain of an inhibitory protein or a portion thereof.

8. The modified immune cell of claim 7, wherein the inhibitory protein is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

9. The modified immune cell of claim 1, wherein the iCAR further comprises a transmembrane domain.

10. The modified immune cell of claim 9, wherein the transmembrane domain is selected from the group consisting of PD-1, CTLA-4, ICOS, LAG-3, TIM3, 2B4, and BTLA.

11. The modified immune cell of claim 1, wherein the immune cell is a T cell.

12. The modified immune cell of claim 11, wherein the T cell is autologous.

13. The modified immune cell of claim 11, wherein the T cell is allogeneic.

14. A nucleic acid encoding an inhibitory chimeric antigen receptor (iCAR), wherein the iCAR comprises an antigen-binding domain capable of binding a pericyte-associated or pericyte-specific antigen, and an inhibitory signaling domain.

15. The nucleic acid of claim 14, wherein the iCAR further comprises a transmembrane and/or a hinge domain.

16. The nucleic acid of claim 14, wherein the antigen-binding domain is an antibody or an antigen-binding fragment thereof.

17. The nucleic acid of claim 16, wherein the antigen-binding fragment is a Fab or an scFv.

18. The nucleic acid of claim 14, wherein the antigen-binding domain is an extracellular domain of a pericyte-associated or pericyte-specific ligand.

19. The nucleic acid of claim 14, wherein the antigen-binding domain binds to a surface antigen expressed on the surface of a pericyte, wherein the antigen is not CD19.

20. The nucleic acid of claim 14, wherein the antigen-binding domain targets an antigen selected from the group consisting of CD146, PDGFRB, RGS5, CSPG4, CD248, BGN, FN1, SEMA5A, PLXDC1, THY1, CDH6, TFPI, COL1A2, ITGA1, EDNRA, PCDH18, CDH11, AXL, NTM, TNFRSF1A, S1PR3, and F3.

21. The nucleic acid of claim 14, wherein the inhibitory signaling domain comprises an inhibitory signaling molecule or a portion thereof.

22. The nucleic acid of claim 21, wherein the inhibitory signaling molecule is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

23. An inhibitory chimeric antigen receptor (iCAR) comprising an antigen-binding domain capable of binding a pericyte-associated or a pericyte-specific antigen, and an inhibitory signaling domain.

24. The iCAR of claim 23, wherein the iCAR further comprises a hinge domain.

25. The iCAR of claim 23, wherein the antigen-binding domain is an antibody, or an antigen-binding fragment thereof.

26. The iCAR of claim 25, wherein the antigen-binding fragment is a Fab of scFv.

27. The iCAR of claim 23, wherein the antigen-binding domain is an extracellular domain of a pericyte-associated or pericyte-specific ligand.

28. The iCAR of claim 23, wherein the antigen-binding domain binds to an antigen expressed on the surface of a pericyte, wherein the antigen is not CD19.

29. The iCAR of claim 23, wherein the antigen-binding domain targets an antigen selected from the group consisting of CD146, PDGFRB, RGS5, CSPG4, CD248, BGN, FN1, SEMA5A, PLXDC1, THY1, CDH6, TFPI, COL1A2, ITGA1, EDNRA, PCDH18, CDH11, AXL, NTM, TNFRSF1A, S1PR3, and F3.

30. The iCAR of claim 23, wherein the inhibitory signaling domain comprises an inhibitory signaling molecule or a portion thereof.

31. The iCAR of claim 30, wherein the inhibitory signaling molecule is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

32. The iCAR of claim 23, wherein the iCAR further comprises a transmembrane domain.

33. The iCAR of claim 32, wherein the transmembrane domain is selected from the group consisting of PD-1, CTLA-4, ICOS, LAG-3, TIM3, 2B4, and BTLA.

34. A method of treating cancer in a subject in need thereof, the method comprising administering to the subject a modified T cell comprising a CAR and an iCAR,
wherein the CAR comprises an antigen-binding domain capable of binding CD19, a transmembrane domain, and an intracellular domain, and
wherein the iCAR comprises an antigen-binding domain capable of binding a pericyte-associated or pericyte-specific antigen, and an inhibitory signaling domain.

35. The method of claim 34, wherein the administering further prevents neurotoxicity in the subject.

36. A method of inhibiting CAR T cell-induced neurotoxicity, the method comprising administering to the subject an effective amount of a modified T cell comprising a CAR and an iCAR,
wherein the CAR comprises an antigen-binding domain capable of binding CD19, a transmembrane domain, and an intracellular domain, and
wherein the iCAR comprises an antigen-binding domain capable of binding a pericyte-associated or pericyte-specific antigen, and an inhibitory signaling domain.

37. The method of claim 34, wherein the antigen-binding domain of the CAR and/or iCAR is an antibody or an antigen-binding fragment thereof.

38. The method of claim 37, wherein the antigen-binding fragment is a Fab or an scFv.

39. The method of claim 34, wherein the antigen-binding domain of the iCAR is an extracellular domain of a pericyte-associated or pericyte-specific ligand.

40. The method of claim 34, wherein the antigen-binding domain of the iCAR binds to an antigen expressed on the surface of a pericyte, wherein the antigen is not CD19.

41. The method of claim 34, wherein the antigen-binding domain of the iCAR binds to an antigen selected from the group consisting of CD146, PDGFRB, RGS5, CSPG4, CD248, BGN, FN1, SEMASA, PLXDC1, THY1, CDH6, TFPI, COL1A2, ITGA1, EDNRA, PCDH18, CDH11, AXL, NTM, TNFRSF1A, S1PR3, and F3.

42. The method of claim 34, wherein the inhibitory signaling domain of the iCAR comprises an inhibitory signaling domain of an inhibitory protein or a portion thereof.

43. The method of claim 42, wherein the inhibitory protein is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

44. The method of claim 34, wherein the iCAR further comprises a transmembrane domain.

45. The method of claim 44, wherein the transmembrane domain is selected from the group consisting of CD4, CD8, CTLA-4, PD-1, ICOS, LAG-3, 2B4, and BTLA.

46. The method of claim 34, wherein the T cell is autologous.

47. The method of claim 34, wherein the T cell is allogeneic.

48. The method of claim 34, wherein the subject is human.

49. A modified immune cell comprising a chimeric antigen receptor (CAR) and an inhibitory CAR (iCAR),
wherein the CAR comprises an antigen-binding domain capable of binding CD19, a transmembrane domain, and an intracellular domain, and
wherein the iCAR comprises an antigen-binding domain capable of binding a mural cell-associated or mural cell-specific antigen, and an inhibitory signaling domain.

50. The modified immune cell of claim 49, wherein the antigen-binding domain of the CAR and/or iCAR is an antibody or an antigen-binding fragment thereof.

51. The modified immune cell of claim 50, wherein the antigen-binding fragment is a Fab or a scFv.

52. The modified immune cell of claim 49, wherein the antigen-binding domain of the iCAR is an extracellular domain of a mural cell-associated or mural cell-specific ligand.

53. The modified immune cell of claim 49, wherein the antigen-binding domain of the iCAR binds to an antigen expressed on the surface of a mural cell, wherein the antigen is not CD19.

54. The modified immune cell of claim 49, wherein the antigen-binding domain of the iCAR binds to an antigen selected from the group consisting of CD146, PDGFRB, RGS5, CSPG4, CD248, BGN, FN1, SEMASA, PLXDC1, THY1, CDH6, TFPI, COL1A2, ITGA1, EDNRA, PCDH18, CDH11, AXL, NTM, TNFRSF1A, S1PR3, and F3.

55. The modified immune cell of claim 49, wherein the inhibitory signaling domain of the iCAR comprises an inhibitory signaling domain of an inhibitory protein or a portion thereof.

56. The modified immune cell of claim 55, wherein the inhibitory protein is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

57. The modified immune cell of claim 49, wherein the iCAR further comprises a transmembrane domain.

58. The modified immune cell of claim 57, wherein the transmembrane domain is selected from the group consisting of PD-1, CTLA-4, ICOS, LAG-3, TIM3, 2B4, and BTLA.

59. The modified immune cell of claim 49, wherein the immune cell is a T cell.

60. The modified immune cell of claim 59, wherein the T cell is autologous.

61. The modified immune cell of claim 59, wherein the T cell is allogeneic.

62. A nucleic acid encoding an inhibitory chimeric antigen receptor (iCAR), wherein the iCAR comprises an antigen-binding domain capable of binding a mural cell-associated or mural cell-specific antigen, and an inhibitory signaling domain.

63. The nucleic acid of claim 62, wherein the iCAR further comprises a transmembrane and/or a hinge domain.

64. The nucleic acid of claim 62, wherein the antigen-binding domain is an antibody or an antigen-binding fragment thereof.

65. The nucleic acid of claim 64, wherein the antigen-binding fragment is a Fab or an scFv.

66. The nucleic acid of claim 62, wherein the antigen-binding domain is an extracellular domain of a mural cell-associated or mural cell-specific ligand.

67. The nucleic acid of claim 62, wherein the antigen-binding domain binds to a surface antigen expressed on the surface of a mural cell, wherein the antigen is not CD19.

68. The nucleic acid of claim 62, wherein the antigen-binding domain targets an antigen selected from the group consisting of CD146, PDGFRB, RGS5, CSPG4, CD248, BGN, FN1, SEMASA, PLXDC1, THY1, CDH6, TFPI, COL1A2, ITGA1, EDNRA, PCDH18, CDH11, AXL, NTM, TNFRSF1A, S1PR3, and F3.

69. The nucleic acid of claim 62, wherein the inhibitory signaling domain comprises an inhibitory signaling molecule or a portion thereof.

70. The nucleic acid of claim 69, wherein the inhibitory signaling molecule is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

71. An inhibitory chimeric antigen receptor (iCAR) comprising an antigen-binding domain capable of binding a mural cell-associated or a mural cell-specific antigen, and an inhibitory signaling domain.

72. The iCAR of claim 71, wherein the iCAR further comprises a hinge domain.

73. The iCAR of claim 71, wherein the antigen-binding domain is an antibody, or an antigen-binding fragment thereof.

74. The iCAR of claim 73, wherein the antigen-binding fragment is a Fab of scFv.

75. The iCAR of claim 71, wherein the antigen-binding domain is an extracellular domain of a mural cell-associated or mural cell-specific ligand.

76. The iCAR of claim 71, wherein the antigen-binding domain binds to an antigen expressed on the surface of a mural cell, wherein the antigen is not CD19.

77. The iCAR of claim 76, wherein the antigen-binding domain targets an antigen selected from the group consisting of CD146, PDGFRB, RGS5, CSPG4, CD248, BGN, FN1, SEMA5A, PLXDC1, THY1, CDH6, TFPI, COL1A2, ITGA1, EDNRA, PCDH18, CDH11, AXL, NTM, TNFRSF1A, S1PR3, and F3.

78. The iCAR of claim 77, wherein the inhibitory signaling domain comprises an inhibitory signaling molecule or a portion thereof.

79. The iCAR of claim 78, wherein the inhibitory signaling molecule is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

80. The iCAR of claim 71, wherein the iCAR further comprises a transmembrane domain.

81. The iCAR of claim 80, wherein the transmembrane domain is selected from the group consisting of PD-1, CTLA-4, ICOS, LAG-3, TIM3, 2B4, and BTLA.

82. A method of treating cancer in a subject in need thereof, the method comprising administering to the subject a modified T cell comprising a CAR and an iCAR,
wherein the CAR comprises an antigen-binding domain capable of binding CD19, a transmembrane domain, and an intracellular domain, and
wherein the iCAR comprises an antigen-binding domain capable of binding a mural cell-associated or mural cell-specific antigen, and an inhibitory signaling domain.

83. The method of claim 82, wherein the administering further prevents neurotoxicity in the subject.

84. A method of inhibiting CAR T cell-induced neurotoxicity, the method comprising administering to the subject an effective amount of a modified T cell comprising a CAR and an iCAR,
wherein the CAR comprises an antigen-binding domain capable of binding CD19, a transmembrane domain, and an intracellular domain, and
wherein the iCAR comprises an antigen-binding domain capable of binding a mural cell-associated or mural cell-specific antigen, and an inhibitory signaling domain.

85. The method of claim 82, wherein the antigen-binding domain of the CAR and/or iCAR is an antibody or an antigen-binding fragment thereof.

86. The method of any one of claim 85, wherein the antigen-binding fragment is a Fab or an scFv.

87. The method of claim 82, wherein the antigen-binding domain of the iCAR is an extracellular domain of a mural cell-associated or mural cell-specific ligand.

88. The method of claim 82, wherein the antigen-binding domain of the iCAR binds to an antigen expressed on the surface of a mural cell, wherein the antigen is not CD19.

89. The method of claim 82, wherein the antigen-binding domain of the iCAR binds to an antigen selected from the group consisting of CD146, PDGFRB, RGS5, CSPG4, CD248, BGN, FN1, SEMASA, PLXDC1, THY1, CDH6, TFPI, COL1A2, ITGA1, EDNRA, PCDH18, CDH11, AXL, NTM, TNFRSF1A, S1PR3, and F3.

90. The method of claim 82, wherein the inhibitory signaling domain of the iCAR comprises an inhibitory signaling domain of an inhibitory protein or a portion thereof.

91. The method of claim 90, wherein the inhibitory protein is selected from the group consisting of PD-1, CTLA-4, ICOS, LAG-3, 2B4, BTLA, CD45, CD148, RPTPa, RPTPk, LAR, LYP/Pep, PTP-PEST, SUP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and any combination thereof.

92. The method of claim 82, wherein the iCAR further comprises a transmembrane domain.

93. The method of claim 92, wherein the transmembrane domain is selected from the group consisting of CD4, CD8, CTLA-4, PD-1, ICOS, LAG-3, 2B4, and BTLA.

94. The method of claim 82, wherein the T cell is autologous.

95. The method of claim 82, wherein the T cell is allogeneic.

96. The method of claim 82, wherein the subject is human.

97. A bi-specific iCAR comprising:
a) a CAR comprising an antigen-binding domain capable of binding CD19, a transmembrane domain, an intracellular domain, and a CD3zeta domain, and
b) an iCAR comprising an antigen-binding domain capable of binding a mural cell, a transmembrane domain, and an inhibitory signaling domain.

98. The method of claim 97, wherein the inhibitory signaling domain of the iCAR is selected from the group consisting of PTPN6, LAIR1, PD-1, and/KIR2DL4.
